(19) United States
(12) Patent Application Publication
(10) Pub. No.: US 2025/0255521 A1
(43) Pub. Date: Aug. 14, 2025

(54) SAMPLE COLLECTION SYSTEM

(71) Applicant: BECTON, DICKINSON AND COMPANY, Franklin Lakes, NJ (US)

(72) Inventors: Adam Steel, Fallston, MD (US); Craig Russ, Wayne, NJ (US); Michael R. Ross, Wayne, NJ (US); Ashay Alur, Bloomingdale, NJ (US); Jason Nikitczuk, Hillsborough, NJ (US)

(73) Assignee: BECTON, DICKINSON AND COMPANY, Franklin Lakes, NJ (US)

(21) Appl. No.: 18/881,785

(22) PCT Filed: Jul. 7, 2023
(86) PCT No.: PCT/US2023/027152

§ 371 Date: Jan. 7, 2025

Related U.S. Application Data
(60) Provisional application No. 63/359,373, filed on Jul. 8, 2022.

(51) Int. Cl. A61B 5/15; A61B 5/154

(52) U.S. Cl. CPC A61B 5/15003; A61B 5/150259; A61B 5/150343; A61B 5/150732; A61B 5/154

(57) ABSTRACT

Sample collection systems are provided. In one aspect, a sample collection system comprises a container, an adaptor and a securement. The adaptor receives a first end of the container into an interior of the adaptor. The securement holds the container at a first position, where the first end of the container is thereby held in a spaced relationship with a needle of the adaptor. The securement is operated or operates to permit the container to be advanced distally within the interior of the adaptor from the first position to a second position to engage the needle to receive a sample in the container. The securement further permits the container to be withdrawn proximally to remove the container from the interior of the adaptor, wherein, when the container is removed from the interior of the adaptor, the securing mechanism is removed by the container.

### CROSS REFERENCE TO RELATED APPLICATIONS

This application claims the priority of and benefit from U.S. Provisional Patent Application No. 63/359,373, filed Jul. 8, 2022, which is incorporated by reference herein.

### TECHNICAL FIELD

The present technology relates to a sample collection system having an adaptor and a disposable sample collection container into which an initial portion of the patient sample is collected. Using venipuncture, the adaptor is fluidically coupled with the vein of patient. Initially, the patient sample is drawn through the adaptor and into the disposable collection system. Once the initial portion of the sample is collected, the disposable sample collection container and a mechanism that holds the disposable sample collection container in the adaptor is discarded and the adaptor is fluidically coupled to a sample collection vessel for collecting the remaining portion of the patient sample.

### BACKGROUND

A blood culture test is presently the preferred method for identifying bacteremia and septicemia (sepsis). Sepsis is a body-wide response to a bacterial infection of the blood stream that can cause organ failure and death. Sepsis kills every one in six infected patients. Moreover, half of all in-hospital deaths involve sepsis. In fact, sepsis kills more people than AIDS, breast cancer and prostate cancer combined. Sepsis affects more hospital patients than any other diagnosis. Given the prevalence of sepsis, a patient's blood is frequently monitored to determine if there are bacteria in the patient's blood that indicates the onset of sepsis.

The need to accurately and reliably determine whether or not a patient has sepsis is apparent. Unfortunately, the United States healthcare system spends over $4 billion each year on unnecessary treatment associated with false positive blood culture results. See Oren Zwang & Richard K. Albert, Analysis of Strategies to Improve Cost Effectiveness of Blood Cultures, 1 J. Hosp. Med. 272 (September 2006). Moreover, “[i]t is currently accepted that most organisms identified as contaminants in blood cultures originate from the skin of the patient.” Robert A. Garcia et al., Multidisciplinary Team Review of Best Practices for Collection and Handling of Blood Cultures to Determine Effective Interventions for Increasing the Yield of True-Positive Bacteremia, Reducing Contamination, and Eliminating False-Positive Central Line-Associated Bloodstream Infections, 43 Am. J. Infect. Control 1222 (November 2015).

Thus, during a blood collection process, there is a need for a device capable of capturing an initial flow of blood from a patient that might contain contaminants from the skin of that patient in order to prevent that portion of the collected sample from entering the blood culture. Capturing the initial portion of the collected blood may reduce the number of collection positives. One such device is described in WO2019018324 to Milan Ivosevic, which was filed on Jul. 17, 2018 as PCT/US2018/042367 and is incorporated in its entirety by reference herein. Another such device is described in U.S. Provisional Application No. 62/883,941, which was filed on Aug. 7, 2019 and is incorporated in its entirety by reference herein.

### BRIEF SUMMARY

A sample collection system is described herein. The system includes a container for collecting a sample; an adaptor having a distal end, a proximal end, a needle, and an interior. The adaptor is configured to receive sample through a connector at the distal end. The sample may be collected via venipuncture or via a venous catheter. The needle extends from the connector into the interior of the adaptor, and the proximal end includes an opening to receive a first end of the container into the interior of the adaptor. The system also includes a securing mechanism configured to releasably secure the container at a first position, wherein, at the first position, the first end of the container is held in a spaced relationship with the needle in the interior of the adaptor. In one aspect, the securing mechanism is operated to allow the container to be advanced distally within the interior of the adaptor from the first position to a second position and thereby engage the needle to receive the sample into the container. In one aspect, the securing mechanism is further configured to enable the container to be withdrawn proximally to remove the container from the interior of the adaptor. In a further aspect, when the container is removed from the interior of the adaptor, the securing mechanism is configured to be removed by the container.

In one aspect, the securing mechanism comprises a collar fitted on the container. In a further aspect, the collar has an inner diameter that is larger than an outer diameter of the container an outer diameter that is smaller than an inner diameter of the adaptor. In a further aspect, the collar provides an interference fit when advanced into the adaptor. In yet a further aspect, the collar has a flange that prevents the collar from being advance completely into the adaptor.

In another aspect, the securing mechanism is a c-clamp. The securing mechanism may also have a resilient gasket that is received on the container and forms an interference fit for the container within the adaptor.

In another aspect, the container has a cap that may have a septum and a shield. The shield may extend from the septum and surrounding a perimeter of a portion of the container. In a further aspect, the above-described shield may have a first O-ring at a proximal end and a second O-ring at a distal end, each O-ring providing an interference fit when in the adaptor.

In another aspect, the securing mechanism is a collar on the container that forms a snap fit with a base of the shield, the securing mechanism providing an interference fit in the adaptor when advanced into the adaptor. In a further aspect, the collar has a flange at the proximal end thereof to prevent the collar from advancing completely into the adaptor.

In another aspect, the adaptor may have a spring on the interior thereof, which provides resistance with the container is advanced into the adaptor and into engagement with the needle, the spring biased to advance the container out of the adaptor after sample collection.

In another aspect of the sample collection system, the securing mechanism is a flanged foam rubber collar paced on the container that forms an interference fit with the adaptor when advanced into the adaptor, the flange having a diameter that is larger than an inner diameter of the adaptor to prevent the flanged collar from being completely advanced into the adaptor.

In another aspect of the sample collection system, the securing mechanism is a foam rubber collar placed on the container that seats within a flanged opening at the proximal end of the adaptor, the foam rubber collar forming an interference fit with the container but permits the container to be advanced and withdrawn through the collar.

In another aspect of the sample collection system, the securing mechanism is a foam rubber collar secured on the container forms interference fit when introduced into the adaptor. In a further aspect, the foam rubber collar is secured on the container with adhesive.

In another aspect of the sample collection system, the securing mechanism may be a flanged, O-ring collar on the container, wherein the O-ring forms a friction fit in the adaptor when the container is advanced in the adaptor and wherein the flange does not advance into the adaptor. In this aspect, the container is movably received in the flanged portion. In another aspect, a sticker is provided to secure the container in its shipment position.

In another aspect, is an alternative mechanism that provides a friction fit for a flange/O-ring assembly that separates the container (i.e., discard tube) from the adaptor needle during shipment. In this aspect, the O-ring is positioned beneath a cap on the container and has a friction fit within the adaptor such that the container is slidably engaged in the adaptor. A sticker is provided to secure the container in its shipment position.

### BRIEF DESCRIPTION OF DRAWINGS

FIG. 1 is a side cross-sectional view of a sample collection system in accordance with the present technology.

FIG. 2A is a side cross-sectional view of another sample collection system in accordance with the present technology.

FIG. 2B is a side perspective view of a c-clamp of the system of FIG. 2A.

FIG. 3A is a side cross-sectional view of another sample collection system in accordance with the present technology.

FIG. 3B is a close-up of a securing mechanism of the system of FIG. 3A.

FIG. 4 is a side cross-sectional view of another sample collection system in accordance with the present technology.

FIG. 5A-FIG. 5D is another aspect of an assembly that keeps the collection/discard tube in spaced relation from the adaptor needle during shipping and storage and will allow the collection/discard tube to advance into engagement with the needle in the adaptor for sample collection. As illustrated, the side views of the sample collection system illustrate a foam rubber flange that provides the desired separation during shipping and storage and stays affixed to the discard tube when removed from the adaptor.

FIG. 6 is another aspect of an assembly that keeps the collection/discard tube in spaced relation from the adaptor needle during shipping and storage and will allow the collection/discard tube to advance into engagement with the needle in the adaptor for sample collection. As illustrated, the side view of the sample/collection discharge tube has a foam rubber flange with a friction fit on the discard tube which is pulled from the adaptor when the tube is removed therefrom. Tabs define a spaced relation between the discard tube and the cap thereon.

FIG. 7 is another aspect of an assembly that keeps the collection/discard tube in spaced relation from the adaptor needle during shipping and storage and will allow the collection/discard tube to advance into engagement with the needle in the adaptor for sample collection. Illustrated in FIG. 7 is an alternative foam flange friction fit to the tube.

FIG. 8A is an alternative mechanism that provides a friction fit for a flange/O-ring assembly that separates the discard tube from the adaptor needle during shipment. In this aspect the O-ring is positioned beneath the cap and has a friction fit within the adaptor such that the discard tube is slidably engaged in the adaptor. A sticker is provided to secure the discard tube in its shipment position.

FIG. 8B is a modified cap or stopper with O-rings disposed in a groove for slidable engagement of the cap in the adaptor. The O-rings provide the friction fit in the adaptor.

FIG. 8C illustrates a sample collection system with the cap from FIG. 8B and a sticker at the flange at the base of the adaptor that secures the collection/discard tube in its spaced relation from the adaptor needle.

FIG. 9A-FIG. 91 illustrate the operation of a sample collection/discard tube in an adaptor. The sample collection/discard tube has fitted thereon a sleeve with flanges to accept an O-ring that secures the sample collection/discard tube from engagement with a needle in the adaptor during shipment but allows for the collection/discard tube to be slid into engagement with the needle for sample collection and then removed from the adaptor after sample collection.

FIG. 10A-FIG. 10B is another aspect of an assembly that keeps the collection/discard tube in spaced relation from the adaptor needle during shipping and storage and will allow the collection/discard tube to advance into engagement with the needle in the adaptor for sample collection. FIG. 10A-FIG. 10B specifically illustrate a rubber ring that fits in the bottom of the adaptor and receives the collection tube snugly to ensure it remains spaced apart from the needle in the adaptor during shipment. The collection/discard tube may be advanced into engagement with the needle for sample collection. After sample collection, the rubber ring is pulled from engagement with the adaptor along with the collection/discard tube.

FIG. 11 is an alternative configuration of the rubber ring of FIG. 10-FIG. 10B. The rubber ring is fitted in the adaptor and the sample collection/discard tube has a flanged ring that engages the rubber ring in the adaptor. The rubber ring is pulled from the adaptor ring by the flanged ring when the collection/discard tube is removed from the adaptor.

FIG. 12 is another alternative configuration of a rubber ring disposed in the bottom the adaptor. In this aspect, the rubber ring has bearings that allow the collection/discard tube to be advanced into engagement with the needle in the adaptor. The rubber ring is prevented from advancing into the adaptor by a flange. The rubber ring is removed from the adaptor when the collection/discard tube cap is advanced out of the adaptor.

FIG. 13A-FIG. 13B is another aspect of an assembly that keeps the collection/discard tube in spaced relation from the adaptor needle during shipping and storage and will allow the collection/discard tube to advance into engagement with the needle in the adaptor for sample collection. FIG. 13A-FIG. 13B illustrate a collar with tabs that provide an interference fit in the adaptor. As illustrated in FIG. 13B, the collar remains in place as the tube is advanced into engagement with the adaptor needle. The collar is removed from the adaptor when the collection/discard tube is pulled from the adaptor.

FIG. 14A-FIG. 14D is another aspect of an assembly that keeps the collection/discard tube in spaced relation from the adaptor needle during shipping and storage and will allow the collection/discard tube to advance into engagement with the needle in the adaptor for sample collection. FIG. 14A-FIG. 14D illustrate a one-way collar with internal grips that provide a friction force that permits the collection/discard tube to be advanced into the adaptor. As illustrated in FIG. 14D, the collar grips the tube such that it is removed from engagement with the adaptor when the collection/discard tube is pulled from the adaptor. The collar has a catch that accepts the flange at the base of the adaptor. The catch is made of a soft material (TPE) so that the seal with the adaptor can be broken and detached from the adaptor.

FIG. 15A-FIG. 15D illustrate an alternative to the collar of FIG. 14A-FIG. 14D. The one-way collar in this aspect has a detent that engages with the base of the adaptor. The collar cannot advance upward into the adaptor. The collar grips the collection/discard tube such that it is removed from engagement with the adaptor when the detent mechanism is placed in the correct orientation to permit the collection/discard tube to be pulled from the adaptor. The grips are directed upwards to further secure the collar on the collection/discard tube.

FIG. 16A-FIG. 16D illustrate an alternative to the collar of FIG. 15A-FIG. 15D with a sticker added to secure the collar to the collection/discard tube.

FIG. 16E-FIG. 16K illustrate the operation of the assembly of FIG. 16A-FIG. 16D. The upward facing grips make it feasible to advance the collection/discard tub upwards into the adaptor but, when the collection/discard tube is advanced out of the adaptor, it carries the collar from the adaptor.

FIG. 17A-FIG. 17B is a two-piece alternative to the collar illustrated in FIG. 13A-FIG. 13B. A collar with barbs is affixed to the collection/discard tube by an interference fit. When advanced into the adaptor the barbs lock into holes in an adaptor collar. When the collection/discard tube is advanced out of the adaptor, it carries the two-piece assembled collar from the adaptor.

FIG. 18A-FIG. 18C is another two-piece assembly for holding the collection/discard tube in spaced arrangement from the needle in the adaptor during shipment but allowing the collection/discard tube to be advanced into engagement with the needle for sample collection. The two-piece assembly is pulled from the adaptor when the collection/discard tube is removed therefrom. A snap ring with upward facing projections is fitted into the base of the adaptor. A sleeve with first and second flanges is fitted on the collection discard tube. When the first flange is advanced above the snap ring, the collection tube is held into place for shipment. When the second flange passes the snap ring, the collection tube is engaged with the needle for sample collection. The second flange on the sleeve pulls the snap ring from the adaptor when the collection/discard tube is removed therefrom.

FIG. 19A-FIG. 19C is an alternative aspect to the two-piece assembly in FIG. 18A-FIG. 18C for holding the collection/discard tube in spaced arrangement from the needle in the adaptor during shipment but allowing the collection/discard tube to be advanced into engagement with the needle for sample collection. The two-piece assembly is pulled from the adaptor when the collection/discard tube is removed therefrom. A snap ring with upward facing projections is fitted into the base of the adaptor. In this aspect the sleeve has first, second, and third flanges on the sleeve fitted on the collection/discard tube. When the first flange is advanced above the snap ring, the collection tube is held into place for shipment. The second flange pushes the snap ring outward when advanced therein, holding the collection/discard tube in place for shipment. When the third flange passes the snap ring, the collection tube is engaged with the needle for sample collection. The third flange on the sleeve pulls the snap ring from the adaptor when the collection/discard tube is removed therefrom.

FIG. 20 is a collection/discard tube with a flanged sleeve thereon for holding the collection/discard tube in spaced arrangement from the needle in the adaptor during shipment but allowing the collection/discard tube to be advanced into engagement with the needle for sample collection. The flanged sleeve prevents the sleeve from advancing beyond the open end of the adaptor when the collection/discard tube is placed therein. The flanged sleeve is flexible. When the collection/discard tube is advanced through the sleeve and into engagement with the needle in the adaptor for blood collection, the sleeve remains in position. Since the sleeve is flexible, it can assist with gripping the collection/discard tube when removed from the adaptor.

FIG. 21 is a two-part collar with a collar that fits in the bottom portion of the adaptor but does not advance therein. The first portion of the collar in the base of the adaptor cooperates with the collar on the collection/discard tube to hold the collection/discard tube in spaced relation to the needle in the adaptor. The second portion of the collar on the collection/discard tube has arms with notched engagement members that will advance past the first portion of the collar as the collection/discard tube is advanced into the adaptor. As such, the second portion of the collar is fashioned as an outside clip. When the collection/discard tube is removed from the adaptor after sample collection, the engagement members on the second portion of the collar pull the first portion of the collar from the base of the adaptor.

FIG. 22 is another flanged sleeve for the collection/discard tube. The flange locks to the flange at the base of the adaptor with a lip that fits over the adaptor base flange. The collection/discard tube is held in spaced arrangement from the needle in the adaptor during shipment by the sleeve. Advancing the collection/discard tube through the sleeve advances the collection/discard tube into connection with the adaptor needle. After the sample is collected, the flange fastener of the sleeve, which is flexible, is detached from the flange at the base of the adaptor and the collection/discard tube, with the sleeve attached, is removed from the adaptor.

FIG. 23A-FIG. 23C is another flanged sleeve that is detached by squeezing the elastomeric sleeve which causes the sleeve flange to separate from the flange at the base of the adaptor when the collection/discard tube is removed from the adaptor. In one aspect the sleeve is affixed to the collection/discard tube by shrink wrap or tape during shipment, which shrink wrap or tape ensures that the collection/discard tube remains spaced apart from the needle during shipment.

FIG. 24A-FIG. 24B is an aspect of what is illustrated in FIG. 23A-FIG. 23C which shows how the sleeve flange seats on the flange at the base of the adaptor to retain the collection/discard tube in the spaced position from the adaptor needle during shipment.

FIG. 25A-FIG. 25B is an aspect of what is illustrated in FIG. 23A-FIG. 23C which shows how the sleeve can be affixed to the collection/discard tube to ensure that the discard collection tube is held in spaced arrangement from the adaptor flange needle during shipment.

FIG. 26 illustrates a collar for the collection/discard tube that has a collapsible section with a living hinge that allows the collar to keep the collection/discard tube in spaced relation to the needle in the adaptor during shipment but, for blood collection, as the collection/discard tube is advanced toward the needle, the living hinge collapses, allowing the collection/discard tube to be advanced into contact with the adaptor needle for blood collection. After collection, the collar is removed with the collection/discard tube as it is removed from the adaptor.

FIG. 27A-FIG. 27B is another aspect of the collection/discard tube assembly wherein the collection/discard tube has a sleeve affixed thereto with a twist-on, twist-off flange that attaches to the flange at the base of the adaptor. As illustrated, the line set is in fluid communication with the adaptor and blood is drawn into the collection/discard tube when the needle in the adaptor pierces the seal in the cap of the collection/discard tube when it is advanced into contact with the needle. The collection/discard tube is advanced in the sleeve, which remains fixed when locked on the flange of the adaptor. When the sleeve is twisted off the adaptor flange, the collection/discard tube is removed from the adaptor.

FIG. 28 is another aspect of the collection/discard tube assembly for holding the collection/discard tube in spaced relation from the needle in the adaptor during shipment but allowing the collection/discard tube to be advanced into engagement with the needle for sample collection In this aspect, the collection/discard tube has a sleeve that can be threaded into the adaptor. There is a flange at the base of the sleeve, which, as illustrated, has helical spines that provide a friction fit when advance into the adaptor. The flange limits how far the sleeve may advance. The sleeve holds the collection/discard tube in spaced relation from the needle during shipment and storage. The collection/discard tube is twisted to advance it within the sleeve. As illustrated, the line set is in fluid communication with the adaptor and blood is drawn into the collection/discard tube when the needle in the adaptor pierces the seal in the cap of the collection/discard tube when it is advanced into contact with the needle. After sample collection, the collection/discard tube is removed from the adaptor.

FIGS. 29A-29C illustrate another aspect of the collection/discard tube assembly for holding the collection/discard tube in spaced relation from the needle in the adaptor during shipment but allowing the collection/discard tube to be advanced into engagement with the needle for sample collection. The collection/discard tube has a hinged spring ring that locks the collection/discard tube in spaced relation from the needle during shipment and storage. The spring flattens into the ring as the collection/discard tube is advanced in the adaptor. Blood is drawn into the collection/discard tube when the needle in the adaptor pierces the seal in the cap of the collection/discard tube when it is advanced into contact with the needle. After sample collection, the collection/discard tube is removed from the adaptor along with the hinged spring.

FIG. 30A-FIG. 30C is another aspect of a two-piece collar/sleeve assembly for holding the collection/discard tube in spaced relation to the needle in the adaptor during shipment and storage but allowing the collection/discard tube to be advanced into engagement with the needle for sample collection wherein the collar has notches. The sleeve has raised guides that travel through the notches in the adaptor collar. The sleeve is fitted onto the collection/discard tube. The sleeve has detents that keep the collection/discard tube in spaced relation from the needle during shipment and storage. Blood is drawn into the collection/discard tube when the needle in the adaptor pierces the seal in the cap of the collection/discard tube when it is advanced into contact with the needle by bypassing the detents. After sample collection, the collection/discard tube is removed from the adaptor along with the collar and sleeve, the sleeve forcing the collar from the adaptor when the collection/discard tube is removed from the adaptor.

FIG. 31A-FIG. 31B is a modified aspect of what is illustrated in FIG. 30A-FIG. 30C. The two-piece collar/sleeve assembly in this aspect also has a collar has notches. The collar has clips that fasten to the flange at the base of the adaptor. The sleeve has raised guides that travel through the notches in the adaptor collar. The sleeve is fitted onto the collection/discard tube. The sleeve has raised portions at the base of the sleeve that keep the sleeve from advancing too far into the adaptor collar and ensures that the collection/discard tube is held in spaced relation from the needle during shipment and storage. Blood is drawn into the collection/discard tube when the needle in the adaptor pierces the seal in the cap of the collection/discard tube when it is advanced into contact with the needle by bypassing the detents. After sample collection, the collection/discard tube is removed from the adaptor along with the collar and sleeve, the sleeve forcing the collar from the adaptor when the collection/discard tube is removed from the adaptor.

FIG. 32A-FIG. 32B is a modified aspect of an assembly for holding the collection/discard tube in spaced arrangement from the needle in the adaptor during shipment but allowing the collection/discard tube to be advanced into engagement with the needle for sample collection. In this aspect, the collar that is disposed/secured on the collection/discard tube has posts that engage a slot in the adaptor housing. The side slot allows the collar to be rotated into a locked position that locks the collection/discard tube in spaced relation from the needle during shipment and storage. To advance the collection/discard tube into engagement with the adaptor needle, the collection/discard tube is rotated and the collar/collection/discard tube assembly can be advanced into engagement with the needle in the adaptor for blood collection because the collar pin can again advance along the slot in the adaptor housing. After sample collection, the collection/discard tube is removed from the adaptor along with the collar.

FIG. 33A-FIG. 33B is a modification of the aspect of FIG. 32A-FIG. 32B. The collar that is disposed on the collection/discard tube has posts that engage a slot in the adaptor housing. The side slot allows the collar to be rotated into a locked position that locks the collection/discard tube in spaced relation from the needle during shipment and storage. The side slot is angled downward, drawing the collection/discard tube slightly out of the adaptor as the pin advances in the detent slot. To advance the collection/discard tube into engagement with the adaptor needle, the collection/discard tube is rotated and the collar/collection/discard tube assembly can be advanced into engagement with the needle in the adaptor for blood collection because the collar pin can again advance along the slot in the adaptor housing. After sample collection, the collection/discard tube is removed from the adaptor along with the collar.

FIG. 34A-FIG. 34B is a modification of aspects of FIG. 32A-FIG. 32B. The collar that is disposed on the collection/discard tube has posts that engage a slot in the adaptor housing. The side slot allows the collar to be rotated into a locked position that locks the collection/discard tube in spaced relation from the needle during shipment and storage. To advance the collection/discard tube into engagement with the adaptor needle, the collection/discard tube is rotated and the collar/collection/discard tube assembly can be advanced into engagement with the needle in the adaptor for blood collection because the collar pin can again advance along the slot that is adjacent to the side slot in the adaptor housing. After sample collection, the collection/discard tube is again rotated and the collar pin travels down a discard slot, thereby removing the collection/discard tube from the adaptor along with the collar.

FIG. 35A-FIG. 35B is a modification of the aspects of FIG. 32A-FIG. 32B. In this aspect, the adaptor has a collar with the detent pin and the collection/discard tube has slots that engage the detent posts and control the motion of the collection/discard tube relative to the adaptor. The ribbed sleeve fits snugly on the collection/discard tube. When the collection/discard tube is rotated such that the detent post is engaged in the side slot, this locks the collection/discard tube in spaced relation from the needle during shipment and storage. To advance the collection/discard tube into engagement with the adaptor needle, the collection/discard tube is rotated and the collar/collection/discard tube assembly may be advanced into engagement with the needle in the adaptor for blood collection because the collar pin can again advance along the slot in the sleeve. After sample collection, the collection/discard tube is again rotated and the sleeve travels downward because the collar pin is aligned with the slot in sleeve. When the collection discard tube is removed from the adaptor, it engages the adaptor collar, pulling it from the adaptor.

FIG. 36A-FIG. 36B is another aspect of an assembly that keeps the collection/discard tube in spaced relation from the adaptor needle during shipping and storage and will allow the collection/discard tube to advance into engagement with the needle in the adaptor for sample collection. In the illustrated aspect, the adaptor has a sleeve that latches into openings in the adaptor housing. The collection/discard tube has a collar with flexible projections that align with grooves in the adaptor sleeve. When the latch in the sleeve is depressed, the collection/discard tube may advance into engagement with the needle in the adaptor. At this point, the flexible projections engage openings in the sleeve, causing the collar and sleeve to engage. After sample collection, the collection/discard tube is removed from the adaptor along with the adaptor sleeve.

FIG. 37A-FIG. 37B is another aspect of an assembly that keeps the collection/discard tube in spaced relation from the adaptor needle during shipping and storage and will allow the collection/discard tube to advance into engagement with the needle in the adaptor for sample collection. In the illustrated aspect, the adaptor has a sleeve with a bump off that receive a latch on a collar that is press fit onto the collection/discard tube. When the latch in the collar is depressed, the collection/discard tube may advance into engagement with the needle in the adaptor. The collar also has projections that travel along guides in the adaptor sleeve. At this point, the latch on the collar engages the sleeve. After sample collection, the collection/discard tube is removed from the adaptor along with the adaptor sleeve, which is engaged with the collar.

FIG. 38 is the aspect of FIG. 37A-FIG. 37B but with tape instead of relying on the bump out to lock the collection/discard tube into position during shipping and storage.

FIG. 39A-FIG. 39B is another aspect of an assembly that keeps the collection/discard tube in spaced relation from the adaptor needle during shipping and storage and will allow the collection/discard tube to advance into engagement with the needle in the adaptor for sample collection. In the illustrated aspect, the adaptor has a sleeve with a resilient stop that will allow a projection on a collar press fit onto the collection/discard tube to pass by it when force is applied to advance the collection/discard tube in the adaptor. As such, tape is applied to ensure that the collection/discard tube is retained in the desired spaced relation with the adaptor needle. When the tape is removed, the collection/discard tube may advance into engagement with the needle in the adaptor. The collar also has projections that travel along guides in the adaptor sleeve. At this point, the projections on the collar and the resilient stop engages the projections on the collar. After sample collection, the collection/discard tube is removed from the adaptor along with the adaptor sleeve, which is engaged with the collar.

FIG. 40A-FIG. 40B is another aspect of an assembly that keeps the collection/discard tube in spaced relation from the adaptor needle during shipping and storage and will allow the collection/discard tube to advance into engagement with the needle in the adaptor for sample collection. In the illustrated aspect, the adaptor has sliding groove and locking grooves that cooperate with flexible fingers on the collar affixed to the collection/discard tube. Initially, the flexible fingers are disposed in the locking grooves to ensure that the collection/discard tube is retained in the desired spaced relation with the adaptor needle during shipment and storage. The collection/discard tube is rotated to place the fingers in the sliding grooves of the adaptor and the collection/discard tube may advance into engagement with the needle in the adaptor. After sample collection, the collection/discard tube is removed from the adaptor by sliding it out along the sliding grooves.

FIG. 41 is another aspect of an assembly that keeps the collection/discard tube in spaced relation from the adaptor needle during shipping and storage and will allow the collection/discard tube to advance into engagement with the needle in the adaptor for sample collection. In the illustrated aspect, the collection/discard tube has a sleeve attached thereto with a portion of the sleeve having an enlarged diameter. The adaptor has windows that hold an O-ring in an elliptical configuration. The elliptical O-ring holds the collection/discard tube in spaced relation to the adaptor needle. When the collection/discard tube is forced past the O-ring, the tube can be advanced into contact with the adaptor needle for sample collection. In that position, the wider portion of the sleeve changes the O-ring configuration to circular, allowing the collection/discard container to backed out from the adaptor, carrying the O-ring with it.

FIG. 42A-FIG. 42B is another aspect of an assembly that keeps the collection/discard tube in spaced relation from the adaptor needle during shipping and storage and will allow the collection/discard tube to advance into engagement with the needle in the adaptor for sample collection. In the illustrated aspect, the collection/discard tube has a sleeve attached thereto with a portion of the sleeve having an enlarged diameter. The adaptor has windows above the flange at the base thereof that hold an elliptical ring. The elliptical ring holds the collection/discard tube in spaced relation to the needle adaptor. When the collection/discard tube is forced past the elliptical ring, the tube can be advanced into contact with the adaptor needle for sample collection. In that position, the wider portion of the sleeve changes the elliptical ring configuration to circular, allowing the collection/discard container to backed out from the adaptor, carrying the elliptical ring with it.

FIG. 43A-FIG. 43B is another aspect of an assembly that keeps the collection/discard tube in spaced relation from the adaptor needle during shipping and storage and will allow the collection/discard tube to advance into engagement with the needle in the adaptor for sample collection. In the illustrated aspect, the adaptor has a threaded inner collar with a flange that abuts the flange at the base of the adaptor. The collection/discard tube has a ring with posts. When the collection/discard tube is placed into the adaptor, the collection/discard tube is held away from the adaptor needle. When sample collection is desired, the collection/discard tube is rotated, which advances the ring, threading it such that the ring posts land in the detents in the threaded inner collar. When engaged, the sample is collected. After sample collection, the sample collection/discard tube is pulled from the adaptor with the ring and the threaded inner collar.

FIG. 44A-44B is an alternative aspect of FIG. 43A-FIG. 43B, and is another aspect of an assembly that keeps the collection/discard tube in spaced relation from the adaptor needle during shipping and storage and will allow the collection/discard tube to advance into engagement with the needle in the adaptor for sample collection. In the illustrated aspect, the adaptor has a slotted inner collar with a flange that abuts the flange at the base of the adaptor. The slot has two detents. The collection/discard tube has a ring with posts. When the collection/discard tube is placed into the adaptor, the collection/discard tube is held away from the adaptor needle by the engagement of the post with the first detent. When sample collection is desired, the collection/discard tube is advanced, such that the ring posts land in the locking detents at the top of the adaptor sleeve. When engaged, the sample is collected into the collection/discard tube. After sample collection, the sample collection/discard tube is pulled from the adaptor with the ring and the slotted inner collar.

FIG. 45A-FIG. 45B is another aspect of an assembly that keeps the collection/discard tube in spaced relation from the adaptor needle during shipping and storage and will allow the collection/discard tube to advance into engagement with the needle in the adaptor for sample collection. The adaptor has two retention holes in its housing, proximal to the flange at the bottom of the adaptor. The collection/discard tube has a sleeve with a first larger diameter, a second smaller diameter and a flange distal to the cap of the collection/discard tube. Disposed in the sleeve are fingers that extend outward and will fit in the retention holes in the adaptor housing when aligned therewith and holds the collection/discard tube away from the needle in the adaptor for storage and handling. Retracting the sleeve releases the fingers from the holes in the adaptor housing. The collection/discard tube is then advanced upward for sample collection. After sample collection, the sample collection/discard tube is pulled from the adaptor with the sleeve and fingers affixed thereto.

FIG. 46A-FIG. 46B is another aspect of an assembly that keeps the collection/discard tube in spaced relation from the adaptor needle during shipping and storage and will allow the collection/discard tube to advance into engagement with the needle in the adaptor for sample collection. The adaptor has two retention holes in its housing, proximal to the flange at the bottom the adaptor. The collection/discard tube has a flanged collar thereon and a holster with fingers that extend from the holster upward and outward into the adaptor. As the collar and the collection/discard tube are advanced into the adaptor, the holes in the collar align with the holes in the adaptor and the fingers extending from the holster on the collection/discard tube locks the collection/discard tube in a position away from the needle in the adaptor for storage and handling. Retracting the collar releases the fingers from the holes in the adaptor housing. The collection/discard tube is then advanced upward for sample collection. After sample collection, the sample collection/discard tube is pulled from the adaptor with the holster and fingers affixed thereto.

FIG. 47A-47B illustrates a diversion adaptor that provide a first flow path for collecting a first portion of the collected sample and a second flow path that collects the portion of the sample to be used for testing. The adaptor has an inner sleeve that is retracted into the adaptor when shipped. The inner sleeve is drawn back and posts in the inner sleeve lock into a detent in the adaptor housing and the diversion portion of the sample is drawn through the first flow path in the adaptor into an upper chamber of the adaptor. After the diversion sample is collected, the inner sleeve is advanced upward and the adaptor engages the sample collection bottle, and the sample travels through the second flow path into the sample collection bottle.

FIG. 48A-FIG. 48B illustrate another aspect of an assembly that keeps the collection/discard tube in spaced relation from the adaptor needle during shipping and storage and will allow the collection/discard tube to advance into engagement with the needle in the adaptor for sample collection. In this aspect, the collection/discard tube is delivered into the adaptor within a sleeve that has a flange on the end of the sleeve distal from the adaptor needle. In the first position, when the collection/discard tube is held away from the adaptor needle for shipment and storage, tape secures the sleeve in a position where the sleeve flange is spaced apart from the adaptor flange at the adaptor opening. When the sticker is peeled away, the collection/discard tube and the sleeve are advanced further into the adaptor for sample collection. After sample collection, the sample collection/discard tube is pulled from the adaptor with the sleeve.

FIG. 49A-FIG. 49F illustrate different sticker configurations and placements to maintain the collection/discard tube in spaced relation from the adaptor needle during shipping and storage and will allow the collection/discard tube to advance into engagement with the needle in the adaptor for sample collection upon removal of the sticker.

FIG. 50 illustrates a different sticker configuration and placement to maintain the collection/discard tube in spaced relation from the adaptor needle during shipping and storage and will allow the collection/discard tube to advance into engagement with the needle in the adaptor for sample collection upon removal of the sticker. In this aspect, the collection/discard tube has a collar thereon and the sticker maintains separation of the collar from the adaptor. With the sticker severed, the collar may advance into the adaptor for sample collection. After sample collection, the collection/discard tube along with the sleeve is removed from the adaptor.

FIG. 51 illustrates the use of a heat shrink material to maintain the collection/discard tube in spaced relation from the adaptor needle during shipping and storage and will allow the collection/discard tube to advance into engagement with the needle in the adaptor for sample collection upon removal of the heat shrink material. To advance the collection/discard tube into the adaptor, the heat shrink material is removed. After collection, the collection/discard tube is removed from the adaptor.

FIG. 52 illustrates another aspect of an assembly that keeps the collection/discard tube in spaced relation from the adaptor needle during shipping and storage and will allow the collection/discard tube to advance into engagement with the needle in the adaptor for sample collection. In this aspect, the collection/discard tube is delivered into the adaptor within a sleeve that has a bellows at the end thereof. The bellows operates to keep the collection/discard tube separated from the adaptor needle during shipment and storage. When sample collection is commenced, the collection/discard tube is forced upward, collapsing the bellows and allowing the adaptor needle to insert into the collection/discard tube for sample collection. When the desired amount of sample is collected, the collection/discard tube and sleeve are removed from the adaptor.

FIG. 53 illustrates another aspect of an assembly that keeps the collection/discard tube in spaced relation from the adaptor needle during shipping and storage and will allow the collection/discard tube to advance into engagement with the needle in the adaptor for sample collection. In this aspect, the collection/discard tube is delivered into the adaptor within a sleeve that has a collapsed, corrugated portion. The collapsed, corrugated portion operates to keep the collection/discard tube separated from the adaptor needle during shipment and storage. When sample collection is commenced the corrugated portion unfolds and the collection/discard tube is forced upward, allowing the adaptor needle to insert into the collection/discard tube for sample collection. When the desired amount of sample is collected, the collection/discard tube and sleeve are removed from the adaptor.

FIG. 54 is another aspect of the assembly described in FIG. 52. FIG. 54 illustrates an assembly that keeps the collection/discard tube in spaced relation from the adaptor needle during shipping and storage and will allow the collection/discard tube to advance into engagement with the needle in the adaptor for sample collection. In this aspect, the collection/discard tube is disposed in a sleeve with a bellows at the end thereof, adjacent a flange that does not permit the bellows to enter the adaptor. The bellows operates to keep the collection/discard tube separated from the adaptor needle during shipment and storage. When sample collection is commenced the collection/discard tube is forced upward, collapsing the bellows and allowing the adaptor needle to insert into the collection/discard tube for sample collection. When the desired amount of sample is collected, the collection/discard tube is removed from the adaptor. The sleeve, having never entered the adaptor, remains on the collection/discard tube.

FIG. 55A-FIG. 55B is another aspect of an assembly that keeps the collection/discard tube in spaced relation from the adaptor needle during shipping and storage and will allow the collection/discard tube to advance into engagement with the needle in the adaptor for sample collection. In this aspect, the collection/discard tube is placed in a sleeve with netting disposed on the exterior of the collection/discard tube. Folds in the netting operate to keep the collection/discard tube separated from the adaptor needle during shipment and storage. When sample collection is commenced the collection/discard tube is forced upward, allowing the folds to unfold, thereby allowing the collection/discard tube to engage the adaptor needle, whereby the needle is inserted into the collection/discard tube for sample collection. When the desired amount of sample is collected, the collection/discard tube is removed from the adaptor along with the netting.

FIG. 56 is another aspect of an assembly that keeps the collection/discard tube in spaced relation from the adaptor needle during shipping and storage and will allow the collection/discard tube to advance into engagement with the needle in the adaptor for sample collection. In this aspect, a stopper is placed on top of the cap of the collection/discard tube. The stopper has a perimeter that forms a friction fit with the adaptor when the stopper/collection/discard tube assembly is placed within the adaptor. The friction fit allows the stopper to maintain the collection/discard tube in spaced relation with the adaptor needle.

FIG. 57 is another aspect of an assembly that keeps the collection/discard tube in spaced relation from the adaptor needle during shipping and storage and will allow the collection/discard tube to advance into engagement with the needle in the adaptor for sample collection. In this aspect, a removeable clip, tear-off strip or sticker is used to force a spaced relationship between a sleeve in which the collection/discard tube is disposed and the adaptor. The spaced relationship ensures that the collection/discard tube does not engage the adaptor needle during storage and shipping. When sample collection commences, the clip or sticker is removed and the sleeve in which the collection/discard tube is disposed can be advanced into contact with the adaptor needle for sample collection. After sample collection, the collection/discard tube (still disposed in the sleeve) is removed from the adaptor.

FIG. 58A-FIG. 58C is a three-piece assembly for holding the collection/discard tube in spaced relation from the needle in the adaptor during shipment but allowing the collection/discard tube to be advanced into engagement with the needle for sample collection. The three-piece assembly is pulled from the adaptor when the collection/discard tube is removed therefrom. A snap ring with upward facing projections is fitted into the base of the adaptor. A sleeve with first and second flanges is fitted on the collection/discard tube. A collar is placed adjacent the cap on the collection discard tube, the collar dimensioned to be advanced above the snap ring, which allows for the collection/discard tube to be held spaced apart from the adaptor needle during shipment and storage. When the first flange passes the snap ring, the collection tube is engaged with the needle for sample collection. The first flange on the sleeve and the collar cooperate to pull the snap ring from the adaptor when the collection/discard tube is removed therefrom.

FIG. 59 is a proof of concept illustration of using an elastomeric tube to hold the collection/discard tube in spaced relation from the needle in the adaptor during shipment but allowing the collection/discard tube to be advanced into engagement with the needle for sample collection. When the elastomer tube is removed, the collection/discard tube may be brought into engagement with adaptor needle for sample collection. When the desired amount of sample is collected, the collection/discard tube is removed from the adaptor.

FIG. 60 is a proof of concept illustration of using extensions from a sleeve on a collection/discard tube to hold the collection/discard tube in spaced relation from the needle in the adaptor during shipment but allowing the collection/discard tube to be advanced into engagement with the needle for sample collection. When the collection/discard tube is advanced into engagement with adaptor needle for sample collection, the extensions are carried into the adaptor. When the collection/discard tube is removed from the adaptor, the extensions are carried with it.

FIG. 61 is a further modification of the aspect illustrated in FIG. 60 and also is a proof of concept illustration that uses extensions from a sleeve on a collection/discard tube to hold the collection/discard tube in spaced relation from the needle in the adaptor during shipment but allowing the collection/discard tube to be advanced into engagement with the needle for sample collection. Tape is used to hold the sleeve together. In some aspects, the cardboard is corrugated for additional thickness and sturdiness. When the collection/discard tube is advanced into engagement with the adaptor needle for sample collection, the extensions are carried into the adaptor. When the collection/discard tube is removed from the adaptor, the extensions are carried with it.

FIG. 62 is a further modification of the aspect illustrated in FIG. 60 and also is a proof of concept illustration that uses extensions from a sleeve on a collection/discard tube to hold the collection/discard tube in spaced relation from the needle in the adaptor during shipment but allowing the collection/discard tube to be advanced into engagement with the needle for sample collection. Tape is used to hold the sleeve together and to hold the collection/discard tube in space relation to the adaptor needle. In some aspects, the cardboard is corrugated for additional thickness and sturdiness. When collection is commenced, the tape holding the collection/discard tube in place is removed, allowing the collection/discard tube to be advanced into engagement with the adaptor for sample collection, the extensions are carried into the adaptor. When the collection/discard tube is removed from the adaptor, the extensions are carried with it.

FIG. 63 is a concept illustration of a sleeve structure for the collection/discard tube that is sufficiently wide so as not to enter the adaptor, thereby keeping the collection/discard tube in spaced relation from the needle in the adaptor during shipment but allowing the collection/discard tube to be advanced into engagement with the needle for sample collection. The collection/discard tube can be advanced in the sleeve structure. After sample collection, the structure is squeezed so that the collection/discard tube assembled to the sleeve may be backed out of the adaptor.

FIG. 64 is a concept illustration of a sleeve structure for the collection/discard tube that is sufficiently wide so as not to enter the adaptor, thereby keeping the collection/discard tube in spaced relation from the needle in the adaptor during shipment but allowing the collection/discard tube to be advanced into engagement with the needle for sample collection when the sleeve is squeezed. The collection/discard tube may then be advanced in the sleeve structure. After sample collection, the structure is squeezed for the collection/discard tube assembled to the sleeve to be backed out of the adaptor.

FIG. 65A-FIG. 65B is an assembly with a valve that has a first adaptor for receiving a collection/discard tube and at least one other adaptor for receiving a sample bottle. The assembly has an inlet that may be connected to a line set for collecting a sample from a patient. The valve may direct a first portion of the collected sample into the collection/discard tube. After the desired amount of sample is collected, the valve is rotated, and additional sample is directed to the one or more sample containers.

FIG. 66 is an alternative aspect of what is described in FIG. 65A-FIG. 65B. In this aspect, a pin is provided to prevent the valve from rotating beyond the position that will direct sample beyond the sample collection/discard tube adaptor unless the collection/discard tube is in engagement with the needle in the adaptor.

FIG. 67A-FIG. 67B illustrates an alternative aspect of the assembly described herein that deploys a collection/discard tube in communication with an adaptor for connection to a patient line set to collect sample from a patient into the container. In this aspect, the collection/discard tube is assembled with the adaptor but held in spaced relation from the adaptor needle during shipment and storage. The collection/discard tube may have one of the numerous examples of collars or sleeves or tape described herein for maintaining such spaced relation between the collection/discard tube and the needle. The adaptor has a valve on the end thereof that is placed in connection with the patient line set (not shown). The collection/discard tube is advanced into the adaptor to collect sample, after which the valve is opened. Once the initial portion of the patient sample is collected in the collection/discard tube, the collection/discard tube is removed from the adaptor and then the patient sample collection bottle is placed in fluid communication with adaptor needle for collecting the patient sample into the blood culture container.

FIG. 68A-68B illustrates an alternative aspect of the assembly described herein that deploys a collection/discard tube in communication with an adaptor for connection to a patient line set to collect sample from a patient into the container. In this aspect, the collection/discard tube is assembled with the adaptor but held in spaced relation from the adaptor needle during shipment and storage by a rotating flap. The adaptor has a first port for receiving the collection/discard tube and a second port for receiving the blood culture bottle. The flap moves from a first position that is intermediate between the collection/discard tube and the adaptor needle, thereby preventing the collection/discard tube from coming into contact with the adaptor needle during shipment and storage, to a second position in which the flap is positioned alongside the collection/discard tube, and therefore no longer in the path between the collection/discard tube and the adaptor needle. Advancing the collection/discard tube in the adaptor moves the flap from the first position to the second position. The collection/discard tube is advanced into the adaptor to collect sample, after which the valve is opened. Once the initial portion of the patient sample is collected in the collection/discard tube, the collection/discard tube is removed from the adaptor and then the patient sample collection bottle is placed in fluid communication with the adaptor needle in the larger port for collecting the patient sample into the blood culture container.

FIG. 69A-FIG. 69B illustrates an alternative aspect of the dual port adaptor described in FIG. 68A-FIG. 68B. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a lever/pin assembly with a first position and a second position. In the first position, the lever in the port that receives the collection/discard tube is positioned over a pin in the second port. In this position, the pin cannot move upward, the culture bottle cannot be brought into contact with the needle in the second port. When the collection/discard tube is advanced into fluid communication with the needle in the adaptor, the lever moves away from above the pin and the pin can be move upward, allowing the culture bottle to be placed in fluid communication with the needle in the second port for collection of the remainder of the sample. Once the initial portion of the patient sample is collected in the collection/discard tube, the collection/discard tube is removed from the adaptor and then the patient sample collection bottle is placed in fluid communication with adaptor needle in the larger port for collecting the patient sample into the blood culture container.

FIG. 70A-FIG. 70B illustrates an alternative aspect of the dual port adaptor described in FIG. 69A-FIG. 69B. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a needle shuttle that will move the needle from the first port to the second port. A lock out peg prevents the needle from being moved from the first port to the second port until sample is collected into the collection/discard tube in the first port. When the collection/discard tube is advanced into fluid communication with the needle in the adaptor, the lock out peg is moved upward, which allows the needle shuttle to be moved. Moving the needle shuttle moves the needle from the first port to the second port. The culture bottle can then be placed in fluid communication with the needle in the second port for collecting the remainder of the sample. Once the initial portion of the patient sample is collected in the collection/discard tube, the collection/discard tube is removed from the adaptor and then the patient sample collection bottle is placed in fluid communication with the adaptor needle in the larger port for collecting the patient sample into the blood culture container.

FIG. 71 illustrates an alternative aspect of the dual port adaptor described in FIG. 70A-FIG. 70B. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a needle shuttle that will move the needle from the first port to the second port. In this aspect, the assembly lacks the lock out peg. Once the initial portion of the patient sample is collected in the collection/discard tube, the collection/discard tube is removed from the adaptor, which allows the needle shuttle to be moved from the first port to the second port. and then the patient sample collection bottle is placed in fluid communication with the adaptor needle in the larger port for collecting the patient sample into the blood culture container. Moving the needle shuttle moves the needle from the first port to the second port. The culture bottle can then be placed in fluid communication with the needle in the second port for collection the remainder of the sample.

FIG. 72A-FIG. 72B illustrates an alternative aspect of the dual port adaptor. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a rotary valve that will allow patient sample to flow into the collection/discard tube in one position, and into the blood culture bottle in a second position. Initially, before sample is collected, the rotary valve is in a third position (the “off” position). When the collection/discard tube is brought into fluid communication with the needle in the adaptor, the rotary valve is moved to the first position. Once the initial portion of the patient sample is collected in the collection/discard tube, the collection/discard tube is removed from the adaptor, and the valve is moved to the third position, which allows collecting the patient sample into the blood culture container when the blood culture bottle is brought into fluid communication with the adaptor needle.

FIG. 73 is an alternative aspect of FIG. 72A-FIG. 72B wherein the adaptor has a locking pin that requires the blood culture bottle to be inserted into the adaptor before the rotary valve is moved to the third position.

FIG. 74A-FIG. 74B illustrates an alternative aspect of the dual port adaptor. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The ports are staggered so the collection/discard tube can be placed in one port and the culture bottle can be placed in the other port simultaneously and to provide a differential pressure between the flow path to the collection/discard tube and the flow path to the culture bottle. When the collection/discard tube is brought into fluid communication with the needle in the adaptor, sample is collected into the sample collection/discard tube. Once the initial portion of the patient sample is collected in the collection/discard tube, the blood culture bottle is placed in fluid communication with the needle in the second port. Because the second port is lower than the first port (or, more precisely, because the sample collection tube is held higher in the adaptor than the blood culture vessel) the sample flows preferentially to the blood culture vessel when both the collection/discard tube and the culture bottle are connected in the adaptor.

FIG. 75 is an aspect of a dual port adaptor wherein the sample collection/discard tube is held in spaced relation to the needle in the adaptor by variety of mechanisms described herein such as the rubber ring, the clutch, the sticker or other spacing mechanism. FIG. 75 illustrates a generic separation mechanism integrated with the sliding needle shuttle described in FIG. 71.

FIG. 76 illustrates an adaptor in fluid communication with a sample collection/discard tube. The adaptor has a piezoelectric flow monitor that will indicate to a use when the sample collection discard tube is contains the target amount of sample.

FIG. 77 is an alternative aspect of two port adaptor. Each port is affixed to an end of a “Y” connector. The port that receives the sample collection/discard tube has a retention mechanism that keeps the collection/discard tube in spaced relation from the needle in the adaptor during shipment and storage. When the sample collection/discard tube is brought into fluid communication with the needle in the adaptor port, the line from the line set to the “Y” connector is primed. The sleeve on the needle in the second port acts like a shut off valve when sample is being collected into the sample collection/discard tube. When the culture bottle is placed in fluid communication with the needle in the second port of the adaptor, sample flows preferentially into the culture bottle.

FIG. 78 is an alternative aspect of two port adaptor of FIG. 77 Each port is affixed to an end of a “Y” connector and the ports themselves are arranged in a “Y.” The port that receives the sample collection/discard tube may have a retention mechanism that keeps the collection/discard tube in spaced relation from the needle in the adaptor during shipment and storage. When the sample collection/discard tube is brought into fluid communication with a needle in the adaptor port, the line from the line set to the “Y” connector is primed. The sleeve on the needle in the second port acts like a shut off valve when sample is being collected into the sample collection/discard tube. When the culture bottle is placed in fluid communication with the needle in the second port of the adaptor, sample flows preferentially into the culture bottle since the collection/discard tube has the requisite amount of sample therein.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

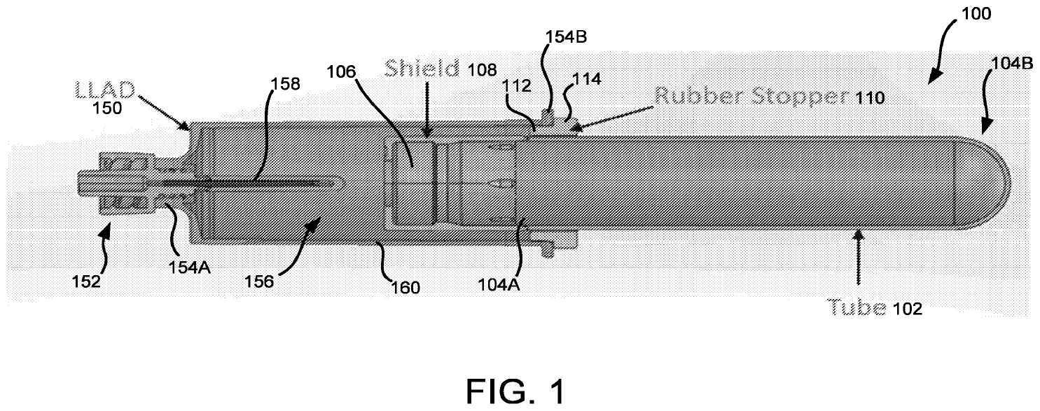

Referring to FIG. 1, a side cross-sectional view of a sample collection system 100 is shown in accordance with the present technology. The sample collected by the system of FIG. 1 may be a fluid, e.g., blood drawn from a patient. The system of FIG. 1 includes a container 102 (e.g., a bottle or tube) for collecting or storing the collected sample and an adaptor 150 configured to fluidically couple a collection container fluidically coupled to the adaptor (e.g., container 102) to a line set (e.g., a venipuncture needle and tubing coupled to a patient) for receiving the sample therefrom. The container 102 is illustrated as a tube having an open end 104A and a closed end 104B. Container 102 further includes a cap or stopper 106 including a septum. The cap 106 is coupled to open end 104A and seals the interior of the container 100 from fluid communication with the environment outside the container 100. Container 102 may further include a shield 108 disposed around the cap 106 to protect the cap 106 from damage during use. Adaptor 150 includes distal end 154A, proximal end 154B, body 160 (e.g., configured in a substantially tubular shape), and needle 158. Adaptor 150 has a connector 152 (illustrated as a luer connector) at the distal end 154A of the adaptor 150. Needle 158 is mounted to, and in fluid communication with, connector 152. Needle 158 extends from connector 152 in a proximal direction into interior 156 of the adaptor 150. Proximal end 154B of adaptor 150 includes an opening providing access to interior 156 and configured to receive end 104A of container 102.

The sample collection is manufactured and shipped in the configuration illustrated in FIG. 1. In this configuration, the container 102 is held separated from and unengaged with needle 158. This is referred to as the first position herein. During the process of collecting a sample, such as blood, from the patient, end 104A of container 102 is advanced further into the interior 156 from end 154B of adaptor 150 from the first position to a second position. As stated above, in the first position, cap 106 is in a spaced relationship (e.g., at a predetermined distance) with the proximal end of needle 158 and not in engagement with needle 158. At the second position, needle 158 engages (i.e., punctures) cap 106 of container 100 (e.g., where needle 158 pierces the septum of cap 106). Furthermore, connector 152 is coupled to a corresponding connector for a line set and a needle of the line set, wherein the line set needle is used for venipuncture to collect a blood sample from a patient. It is to be appreciated that, in some embodiments, connector 152 may be a male Luer-Lok® adaptor or connector connectable to a female Luer-Lok® adaptor of the line set, however other types of connectors for use with adaptors and containers, such as adaptor 150 and container 102, are contemplated to be within the scope of the present disclosure, as are catheters in place of a line set.

Container 102 is a sealed container that has an internal pressure that is either a full or partial vacuum (i.e. sub-atmospheric pressure). When the needle 158 punctures the cap 106, the sub-atmospheric pressure in the interior of container 102, draws an initial portion of blood from the patient via the line set, connector 152, and needle 158 into the interior of container 102. After an initial portion of the patient sample is collected, container 102 is then withdrawn proximally from the interior 156 of adaptor 150 such that needle 158 disengages from fluidic communication with the interior of container 102. Once the needle 158 is no longer in fluid communication with the interior of the container 102, the flow of blood from the patient ceases. Container 102, along with the initial portion of blood collected therein, is then discarded. At this point, a second container (e.g., a blood culture bottle) is advanced into the interior 156 of adaptor 150 to draw blood from the patient. The interior pressure of the second container is also sub-atmospheric. The blood drawn into the second container will serve as the blood that will be subjected to tests to determine if the patient's blood contains microbial contamination.

In some embodiments, the blood collection system of FIG. 1 may be implemented using one of Becton, Dickinson and Company's (“BD's”) Vacutainer® blood collection sets, such as BD's Vacutainer® push button blood collection set, BD's Vacutainer® Safety-Lok™ blood collection set, or BD's Vacutainer® UltraTouch™ push button blood collection set. Therefore, in some embodiments, adaptor 150 may be implemented using BD's Vacutainer® Multiple Sample Luer Adaptor. Moreover, in some embodiments, adaptor 150 may be implemented using BD's Vacutainer® One Use Holder.

As mentioned above, most organisms identified as contaminants in blood cultures originate from the skin of the patient. These contaminants are typically introduced into a patient's blood sample by the venipuncture and the initial flow of blood from the patient into a container or collection bottle, such as container 102. In the sample collection system 100 described herein, the initial portion of the blood collected from a patient is contained in a first container 102 and then discarded. Then, a second portion of the blood collected from the patient is collected into a second container and stored for subsequent testing. As a result, the sample collection system 100 described herein potentially reduces the number of false positive blood cultures by discarding the first portion of the collected sample, which can yield false positives due to the presence of microbial contamination of the sample from the skin of the patient.

In accordance with the present technology, a securing mechanism is provided that is configured to releasably hold or secure a container, such as container 102, to an adaptor, such as adaptor 150 in the first position (i.e., a spaced relationship where container 102 is not in engagement with the needle 158 of the adaptor 150). In this way, the container 102 and adaptor 150 may be shipped to a user or technician as an assembled unit, with the container 102 held in the first position with respect to the adaptor 150 by the securing mechanism. After the technician connects the line set to the patient and the adaptor 150, the securing mechanism is configured to permit the container 102 to be advanced from the first position to the second position. As explained above, in the second position, the needle 158 of the adaptor 150 pierces the seal of the container 102 and the sub-atmospheric pressure in the container 150 draws the patient sample through the line set, the connector/needle 158 and into the container 102. After the blood is collected from the patient into the container 102, the operator can remove the container 102 from the adaptor 150. The securing mechanism is further configured to be removed with the container 102 when the container 102 is removed from the adaptor 150 by the operator.

For example, the system of FIG. 1 may include a securing mechanism 110 configured with the features described above. As shown in FIG. 1, securing mechanism 110 includes a distal end or portion 112 and a proximal end or portion 114. The securing mechanism 110 may be a made of rubber and configured in an annular shape. The securing mechanism 110 is disposed around the exterior of container 102. Distal portion 112 has a predetermined thickness (between an outer diameter of portion 112 and an inner diameter shared by portions 112, 114) selected to enable portion 112 to fit between the exterior of container 102 and an inner wall 160 of the adaptor 150 defining an inner diameter. Furthermore, proximal portion 114 is configured as a rim or flange having a predetermined thickness (between an outer diameter of portion 114 and the inner diameter shared by portions 112, 114) that is selected to be greater than the predetermined thickness of portion 112 such that the outer diameter of portion 114 is greater than the inner diameter of the adaptor 150. In this way, the predetermined thickness of portion 114 ensures that the securing mechanism 110 is retained at the proximal end of the adaptor 150 and does not advance distally into the interior 156 of the adaptor 150.

The predetermined thickness of portion 112 is further configured to provide a sufficient amount of friction between the inner diameter of portion 112 of securing mechanism 110 and the outer diameter of container 102 such that container 102 is releasably secured or held in the first position (shown in FIG. 1) and does not move relative to adaptor 150 absent a force applied to container 102 by a user. From the first position, a user may advance container 102 in a distal direction against the friction applied by securing mechanism 110 into the second position such that the septum seal of cap 106 of the container 102 is urged into engagement with and pierced by needle 158. Since, as described above, the predetermined thickness of portion 114 prevents portion 114 from advancing distally into the interior 156 of adaptor 150, as container 102 is advanced from the first position to the second position, the securing mechanism 110 is held at proximal end 154B of the adaptor 150. After the initial portion of the blood sample has been drawn from the patient user into container 102 (in the manner described above), container 102 is pulled proximally by an operator or user to remove container 102 from the adaptor interior 156. When container 102 is withdrawn from interior 156, securing mechanism 110 remains disposed around the exterior of body 102 and is also pulled from the adaptor 150 by the removal of the container 102 from the adaptor 150.

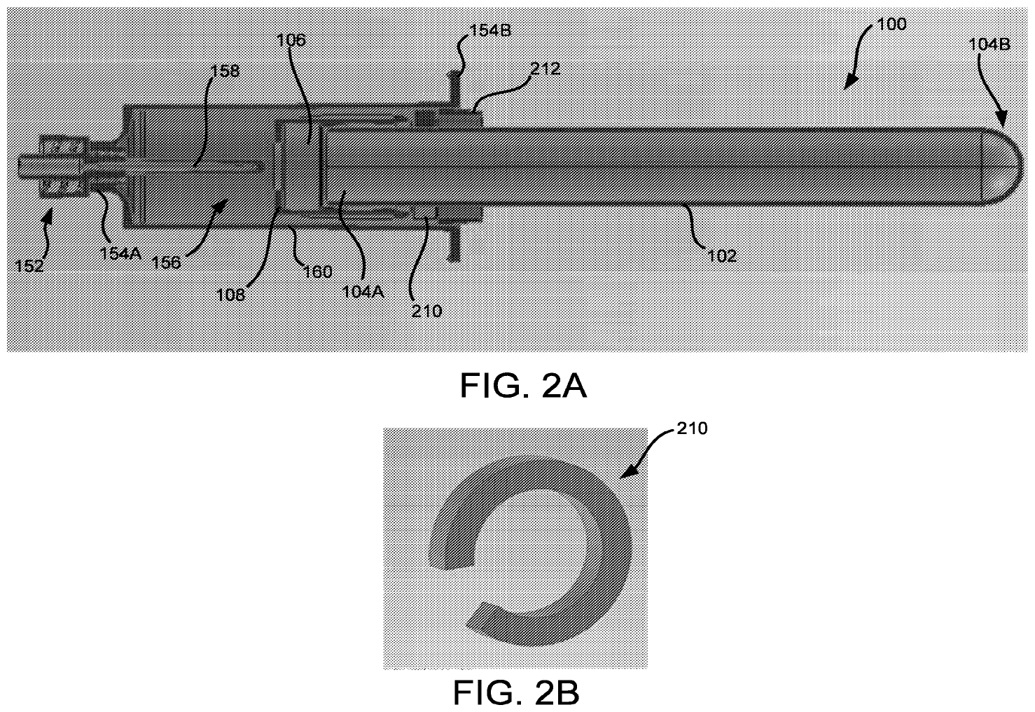

It is to be appreciated that the securing mechanism of the present technology may take other forms in addition to the securing mechanism 110 described above. For example, referring to FIG. 2A, a securing mechanism 210 is shown for use in a sample collection system 100 in accordance with the present technology. Securing mechanism 210 is configured as a c-clamp configured to be disposed around the outer diameter of body 102 of container 100. A gasket, washer or O-ring, etc. 212 is provided in order to provide an interference fit between the adaptor 150 and the container 102. The c-clamp 210 may be made of rubber and includes a predetermined thickness (between in an inner and outer diameter of c-clamp) configured to provide sufficient friction to hold or secure container 100 in the first position (as shown in FIG. 2) so that container 100 does not move from the first position relative to adaptor 150 absent a force applied by a user or technician. As described above, the technician may advance the container 102 distally to the second position after venipuncture such that the line set is in fluid communication with the connector 152 and needle 158 of the adaptor 150. In the second position, the needle 158 pierces the septum seal of the cap 106. The sub-atmospheric pressure in the container 102 draws an initial portion of blood from the patient into the container 102. Thereafter, when a sufficient volume of the initial portion is collected to ensure that microbial skin contamination is collected with the initial portion, the container 102 is pulled proximally by the technician to remove container 102 from interior 156 of adaptor 150. When container 102 is removed from interior 156, c-clamp 210 remains disposed around the outer diameter of container 102, such that the removal of container 102 from interior 156 also removes c-clamp 210 from interior 156. In one aspect, when the container 102 is removed from the adapter, c-clamp 210 and gasket/washer/O-ring 212 are also removed. In accordance with this aspect, the outer diameter of shield 108 may be greater than the inner diameter of c-clamp 210. In this way, when container 102 is withdrawn proximally to remove container 102 from interior 156, a proximal end of shield 108 contacts c-clamp 210 and gasket/washer/O-ring 212 to pull c-clamp 210 and gasket/washer/O-ring 212 from interior 156 of the adaptor along with container 102.

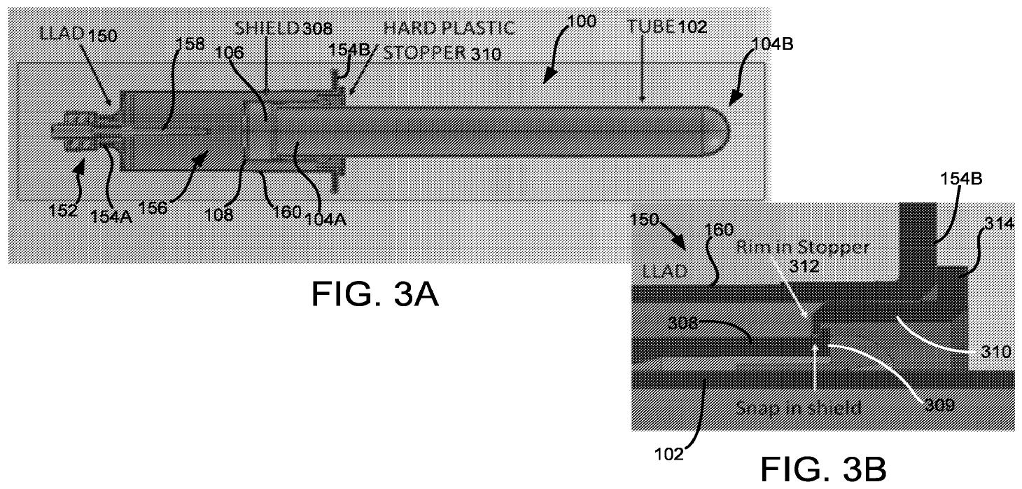

As another example, referring to FIGS. 3A, 3B, a securing mechanism 310 is shown for use in a sample collection system 100 in accordance with the present technology. As shown in FIG. 3A, the securing mechanism 310 may form a snap fit with a shield 308 for container 102. On a proximal end of shield 308, a rim 309 projecting in a direction away from the exterior of container 102. Furthermore, securing mechanism 310 is configured in an annular shape and is disposed around the exterior of container 102. Securing mechanism 310 includes oppositely extending rims 312, 314. Rim 312 is disposed on a distal end of securing mechanism 310 and projects toward the exterior of container 102 (i.e., opposite the direction in which rim 309 projects). Rim 314 is disposed on a proximal end of securing mechanism 310 and projects away from the exterior of container 102. The outer diameter of the securing mechanism 310 from the distal end to before the position of proximal rim 314 is selected to enable the distal portion of securing mechanism 310 to be inserted into interior 156. Rim 314 extends beyond the inner diameter of the adaptor defined by inner surface 160 and functions as a stop or flange at proximal end 154B. In this way, rim 314 prevents securing mechanism 310 from being advanced distally into container 150 beyond where rim 314 is adjacent to and in engagement with the proximal end of the adaptor and holds securing mechanism 310 at proximal end 154B of adaptor 150.

In use, end 104A of container 102 is inserted into interior 156, through an aperture of securing mechanism 310, and advanced distally until rim 312 contacts rim 309 and rim 314 contacts the distal end 154B of the adaptor 150, as shown in FIG. 3B. Rims 312 and 314 are disposed at a predetermined distance from each other, such that, in the arrangement shown in FIG. 3B, container 100 is releasably secured or held in the first position with respect to adaptor 150 by the interlock of rims 309, 312 (and rim 314 abutted against end 154B) preventing container 102 from being advanced distally within interior 156. Rim 312 and/or rim 309 are configured to deform slightly under a predetermined amount of force, such that a user applying a distal force to container 100 can force rim 309 distally beyond rim 312 (which is held stationary relative to adaptor 150 by the interaction of rim 314 with end 154B) to advance container 100 from the first position to the second position such that needle 158 pierces the septum of cap 106. The interaction between rim 314 and distal end 154B retains securing mechanism 310 at the distal end 154B of the adaptor 150 during the distal advancement of container 102. When container 102 is removed from interior 156 by withdrawing container 100 proximally, rim 309 again contacts rim 312 and pulls securing mechanism 310 proximally to remove securing mechanism 310 from adaptor 150. As the container 102 is advanced out of the adaptor 150 rim 314 is advanced away from the proximal end 154B of the adaptor 150.

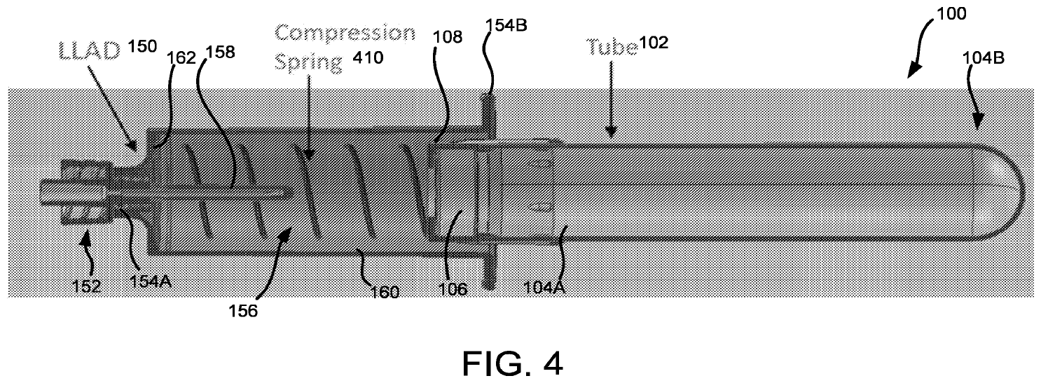

As yet another example, referring to FIG. 4, a securing mechanism 410 is shown for use in a sample collection system 100 in accordance with the present technology. In the system of FIG. 4, securing mechanism 410 is configured as a compressible spring attached to the cap and/or shield 108 at end 104A of container 102. The spring 410 is releasably attached to the interior 156, e.g., to distal surface 162 (shown in FIG. 4), of adaptor 150 and disposed between distal surface 162 and cap 106. Spring 410 is configured to bias container 102 at the first position such that needle 158 and cap 106 are in a spaced relationship and not in engagement. When the system of FIG. 4 is ready for use (i.e., the line set is connected to the patient and connector 152), a technician may advance container 100 from the first position distally against the tension of the spring 410 to the second position, where the needle 158 engages cap 106 (i.e., needle 158 pierces the septum of cap 106) and blood is drawn into container 1020. It is to be appreciated that to maintain the second position and engagement between needle 158 and cap 106, a user must actively hold container 102 in the second position by applying a distal force against container 102 to overcome the tension of spring 410. When the user removes the distal force from container 100, the tension of spring 410 advances container 100 proximally to the second position to automatically disengage cap 106 and needle 158. Thereafter, a user may remove container 100 from interior 156 and, since spring 410 is fixedly attached to cap 106 and/or shield 108, spring 410 is also removed from interior 156 by the removal of container 100.

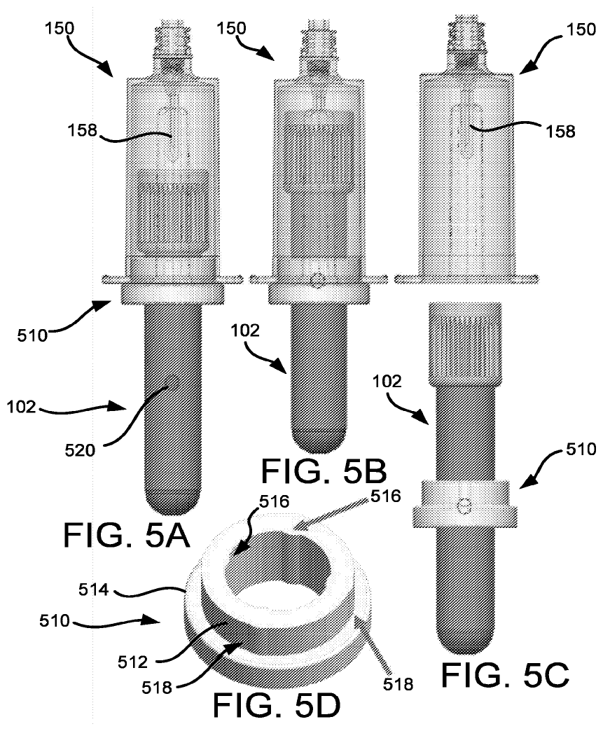

FIG. 5A-FIG. 5D illustrate another aspect of an assembly with a securing mechanism 510 that keeps the collection/discard tube 102 in spaced relation from the adaptor needle 108 during shipping and storage and will allow the collection/discard tube 102 to advance into engagement with the needle 108 in the adaptor 150 for sample collection. As illustrated, the side views in FIGS. 5A-5C of the sample collection system illustrate a foam rubber flange as the securing mechanism 510 that provides the desired separation during shipping and storage and stays affixed to the discard tube 102 when removed from the adaptor 150. In one example, the securing mechanism 510 includes portions 512, 514, which may be configured with the same features described above in relation to portions 112, 114 of securing mechanism 110. In this way, portions 512, 514 of securing mechanism 510 provide an interference fit between the collection/discard tube 102 and the foam 510 and the adaptor 150. The interference fit holds the tube 102 in place relative to the adaptor 150 (e.g., in a spaced relationship in the first position), but allows easy movement of the tube 102 toward the needle 158 upon the application of an advancing force. Portion 514 has a predetermined thickness that is greater than the inner diameter of adaptor 150 to ensure that the securing mechanism 510 is retained at the proximal end of the adaptor 150 and does not advance distally into the interior 156 of the adaptor 150. When the tube 102 is pulled out of adaptor 150, the tube 102 and foam 510 come out of the adaptor 150 together. In one example, securing mechanism 510 includes one or more protrusions 516, 518. The protrusions 516 in the foam 510 are configured to protrude from the inner diameter of foam 510 (defined by the inner diameters of portions 512, 514) diametrically inward to control the interference force between tube 102 and foam 104. The protrusions 518 may also be on the outer diameter of portion 512 on the adaptor side of the foam rubber flange 510. The spot 520 on the collection/discard tube 102 illustrates an optional feature, where a drop of adhesive may be provided to snap into a receiving hole in the foam flange to affix it to the collection/discard tube 102.

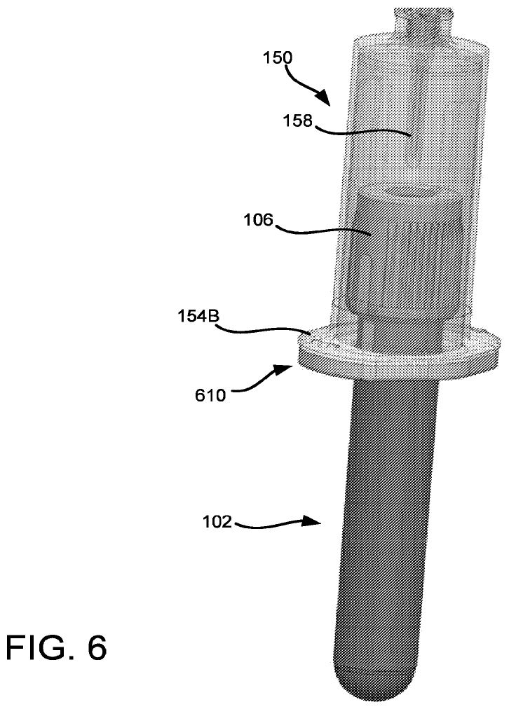

FIG. 6 is another aspect of an assembly with a securing mechanism 610 that keeps the collection/discard tube 102 in spaced relation from the adaptor needle 158 during shipping and storage and will allow the collection/discard tube 102 to advance into engagement with the needle 158 in the adaptor 150 for sample collection. As illustrated, the side view of the sample/collection discharge tube 102 has a foam rubber flange 610 with a friction fit on the discard tube 102, which is pulled from the adaptor 150 when the tube 102 is removed therefrom. The foam rubber flange 610 has a low tack adhesive to affix the flange 610 in the base 154B (e.g., a flange at an end) of the adaptor 150. Tabs define a spaced relation between the discard tube 102 and the cap 106 thereon. In one aspect, the adhesive strength may be selected such that, when the tube 102 is pulled out of adaptor 150, the cap 106 applies a pulling force to the securing mechanism 610 when the cap 106 contacts the securing mechanism 610. The pulling force is sufficient to overcome the adhesion between the securing mechanism 610 and the base or flange 154B to decouple the mechanism 610 from the base or flange 154B. In this way, the securing mechanism 610 is carried away from the adaptor 150 with the removal of tube 102.

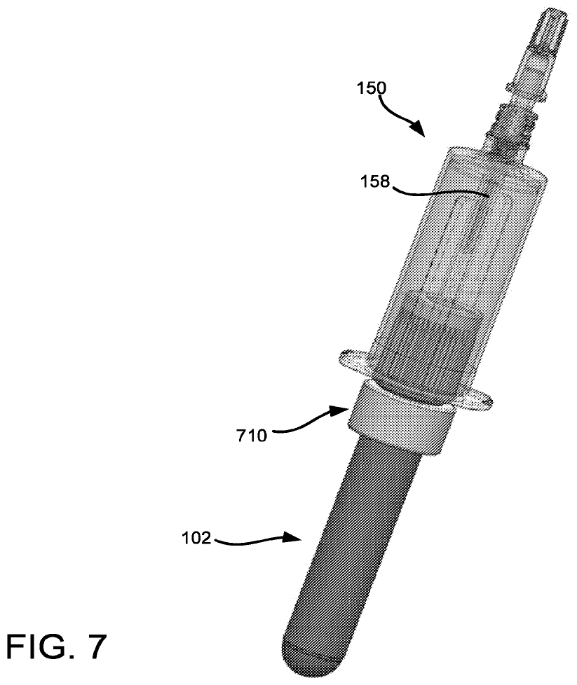

FIG. 7 is another aspect of an assembly with a securing mechanism 710 in accordance with the present disclosure that keeps the collection/discard tube 102 in spaced relation from the adaptor needle 158 during shipping and storage and will allow the collection/discard tube 102 to advance into engagement with the needle 158 in the adaptor 150 for sample collection. Illustrated in FIG. 7 is an alternative foam friction fit collar 710 disposed around the collection/discard tube 102. The collar 710 does not have the flange illustrated in FIG. 5A-FIG. 5D and FIG. 6. The collar 710 is configured with the friction fit features described above in relation to the securing mechanisms described above (e.g., securing mechanisms 110, 510, etc.) In one aspect, the collar 710 may be adhered (e.g., using a low tack adhesive or using one or more stickers, as will be described below) to the exterior of tube 102 to prevent accidental translation of tube 102 prior to a user pushing tube 102 into adapter 150.

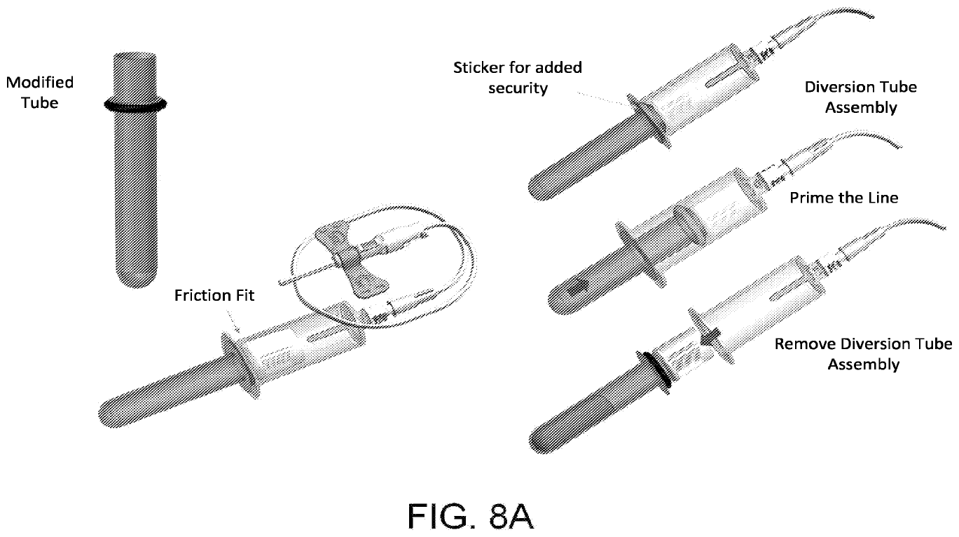

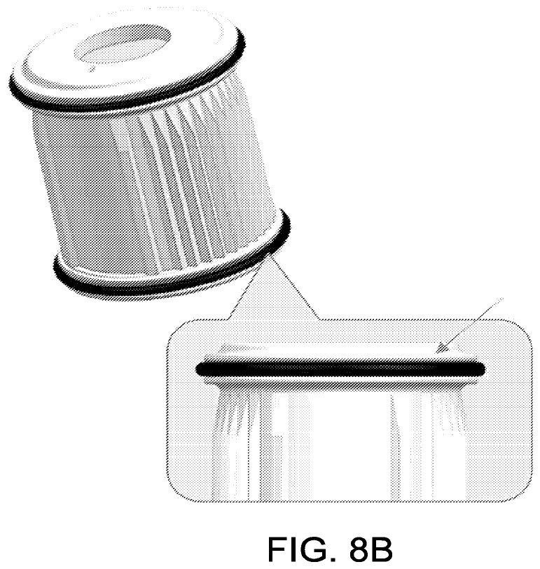

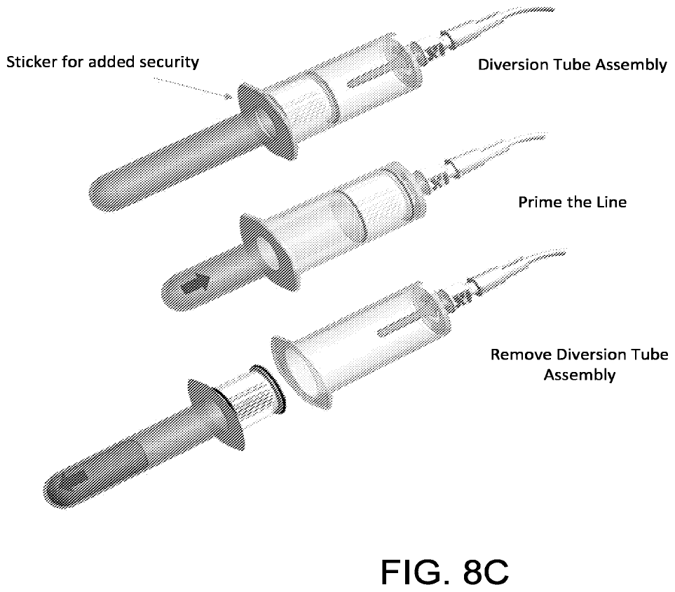

FIG. 8A is an alternative mechanism that provides a friction fit for a flange/O-ring assembly that separates the discard tube from the adaptor needle during shipment. In this aspect, the O-ring is positioned beneath the cap and has a friction fit within the adaptor such that the discard tube is slidably engaged in the adaptor. A sticker is provided to secure the discard tube in its shipment position. The sticker is labeled as such.

FIG. 8B is a modified cap or stopper with O-rings disposed in a groove for slidable engagement of the cap in the adaptor. The O-rings provide the friction fit in the adaptor.

FIG. 8C illustrates a sample collection system with the cap from FIG. 8B and a sticker at the flange at the base of the adaptor that secures the collection/discard tube in its spaced relation from the adaptor needle.

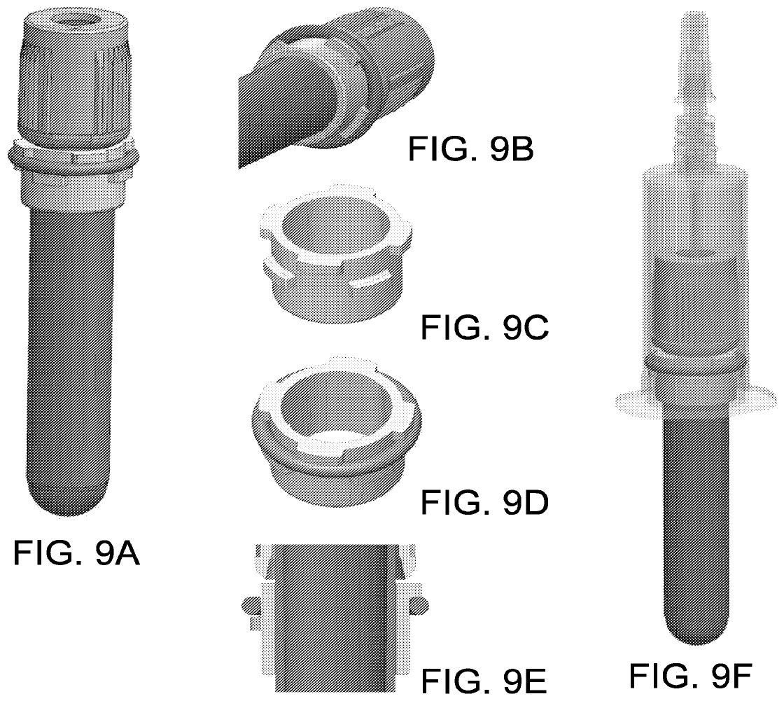

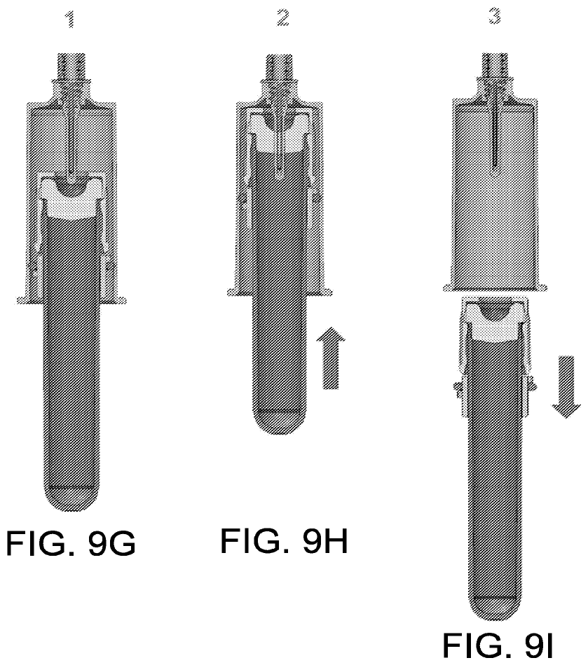

FIG. 9A-FIG. 91 illustrate the operation of a sample collection/discard tube in an adaptor. The sample collection/discard tube has fitted thereon a sleeve with flanges to accept an O-ring that secures the sample collection/discard tube from engagement with a needle in the adaptor during shipment but allows for the collection/discard tube to be slid into engagement with the needle for sample collection and then removed from the adaptor after sample collection.

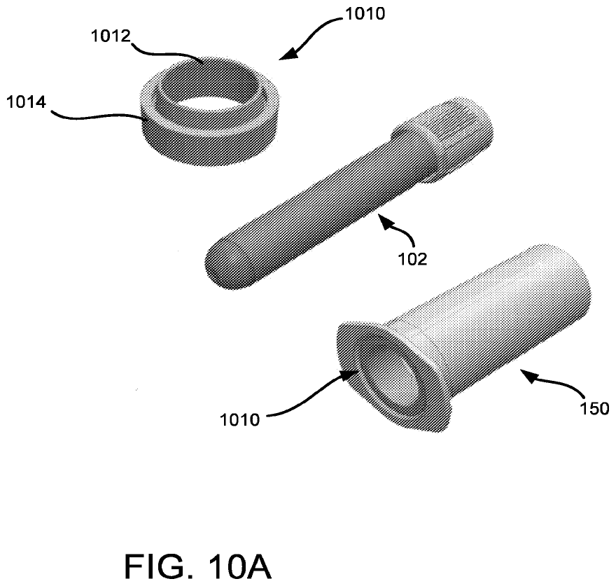

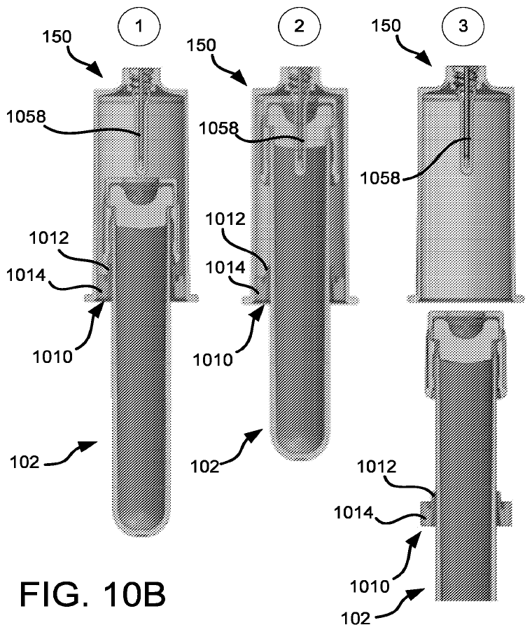

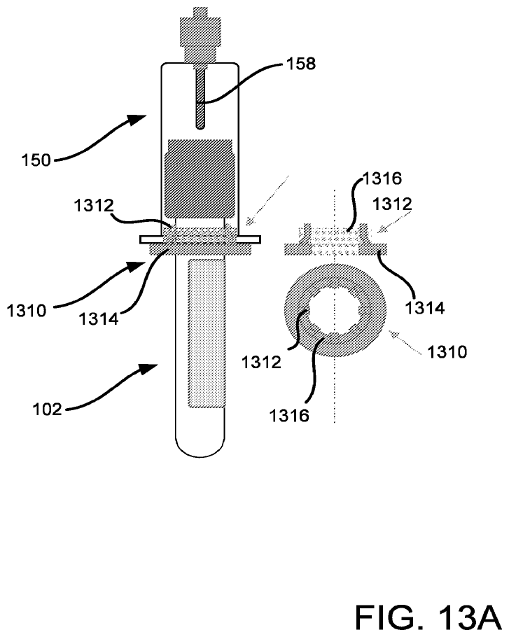

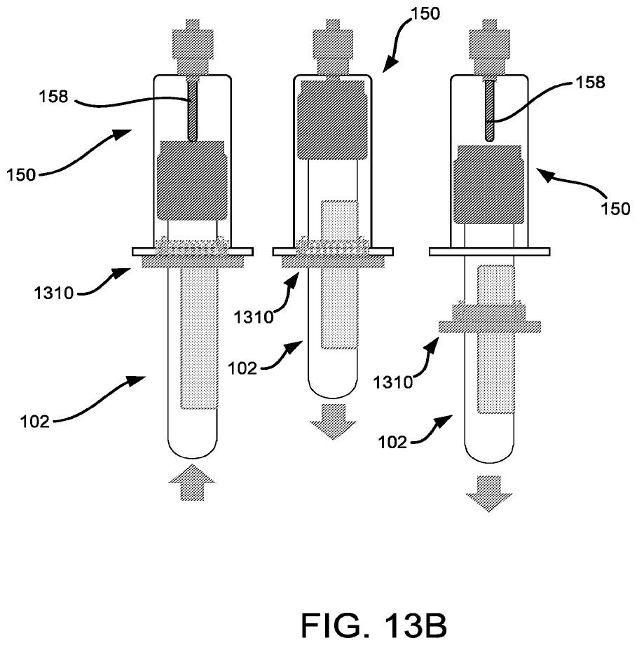

FIG. 10A-FIG. 10B is another aspect of an assembly with a securing mechanism 1010 that keeps the collection/discard tube 102 in spaced relation from the adaptor needle 158 during shipping and storage and will allow the collection/discard tube 102 to advance into engagement with the needle 158 in the adaptor 150 for sample collection. In this regard, the aspects illustrated in FIG. 10A-FIG. 10B are related to the aspects illustrated in FIG. 13A-FIG. 13B. FIG. 10A-FIG. 10B specifically illustrate a ring as the securing mechanism 1010. The ring 1010 may be made of rubber. The rubber ring 1010 includes (annular) portions 1012, 1014. Portion 1014 has a predetermined outer diameter configured to provide an interference fit with the inner diameter of bottom end opening of the adaptor 150. The inner diameter of portion 1012 (which defines the inner diameter of ring 1010) is configured to receive the collection tube 102 snugly to ensure it remains spaced apart from the needle 158 in the adaptor 150 during shipment. In one aspect, portion 1012 projects from portion 1014 distally and has a distally tapering diameter. Portion 1012 is configured (via its inner diameter and shape) to permit the tube 102, upon application of a pushing force, to be advanced easily only in a single direction into adaptor 150 as shown in FIG. 10B such that the collection/discard tube 102 may be advanced into engagement with the needle 158 for sample collection. After sample collection, the rubber ring 1010 is pulled from engagement with the adaptor 150. Upon application of a pulling force on tube 102 to remove tube 102 from adapter 150, portion 1012 grabs or catches onto the exterior of tube 102 and causes the ring 1010 to disengage the adaptor 150 and the tube 102 and ring 1010 pulled together out of the adapter 150. Ring 1010 is configured as a one-way ratchet mechanism.

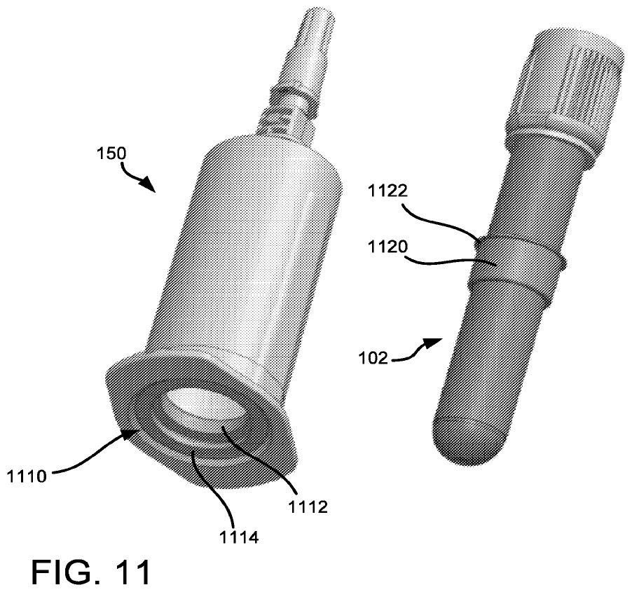

FIG. 11 illustrates securing mechanism 1110, which is an alternative configuration of the rubber ring 1010 of FIG. 10A-FIG. 10B. The rubber ring or securing mechanism 1110 includes portions 1114 and 1112. The exterior diameter of ring 1110 is configured to provide an interference fit with the adapter 150 in the manner described above. Portions 1112 and 1114 each include an inner diameter. The inner diameter of portion 1112 is smaller than portion 1114 and is selected receive and grip the flange 1122 of sleeve 1120 that is disposed on tube 102 to hold tube 102 in a spaced relationship with needle 158 within adapter 150. Tube 102 can be pushed into adapter 150 to engage needle 158. When tube 102 is pulled out of adapter 150, the flange 1122 engages the rubber ring 1110. The rubber ring 1110 is pulled out from the adaptor 150 by the flanged ring 1120 when the collection/discard tube 102 is pulled and removed from the adaptor 150.

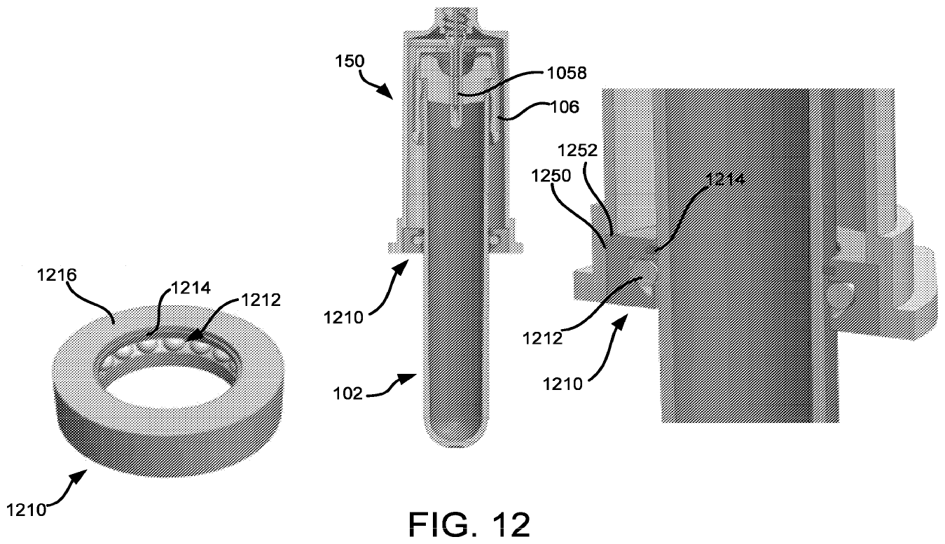

FIG. 12 illustrates a securing mechanism 1210, which is another alternative configuration of a rubber ring disposed in the bottom the adaptor 150 that is a variation of what is described in FIG. 10A-FIG. 10B and FIG. 11. In this aspect, the rubber ring 1210 has bearings 1212 (e.g., ball bearings) that are embedded in an inner circumference of the rubber ring 1210. The ring 1210 has outer and an inner diameters configured to hold the tube 102 in an interference fit with adapter 150. In one aspect, the ring 1210 may include an inner ring 1214 that projects from the inner circumference of ring 1210 toward the center of the central opening of ring 1210. The ring 1214 is configured to grip the exterior of tube 102 to hold tube 102 in place. Upon an application of the pushing force onto tube 102 into adapter 150, the ring 1210 and the bearings 1212 are configured to easily allow the collection/discard tube 102 to be advanced into engagement with the needle 158 in the adaptor 150. In one aspect, the rubber ring 1210 is prevented from advancing into the adaptor 150 by a flange or stepped end 1250 in the inner diameter of the open proximal end 154B of the adapter 150. The stepped end 1250 has an inner diameter that matches the outer diameter of ring 1210. A surface 1216 of ring 1210 abuts against surface or ledge 1252 of stepped end 1250 to prevent ring 1210 from advancing into adapter 150. The rubber ring 1210 is removed from the adaptor 150 when the tube 102 is pulled or advanced out of the adaptor 150. In one aspect, when the tube 102 is pulled out of the adaptor 150, the ring 1210 (e.g., due to the gripping ring 1214 and the surrounding material of ring 1212) is configured to pinch the ball bearing 1212 such that the gripping force of ring 1210 on tube 102 is increased and the ring 1210 disengages the adaptor 150 and is pulled out of the adaptor 150 with the tube 102.

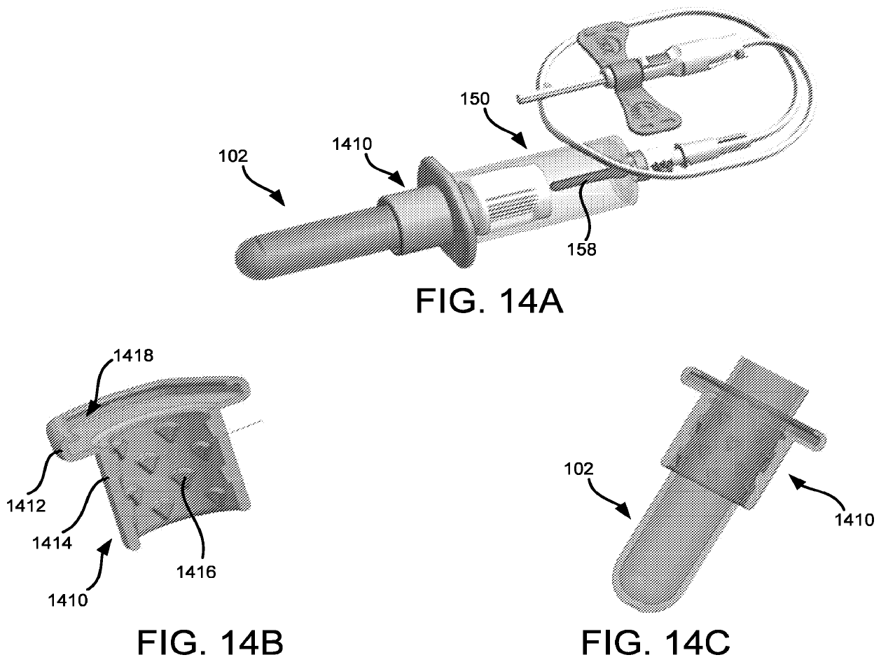

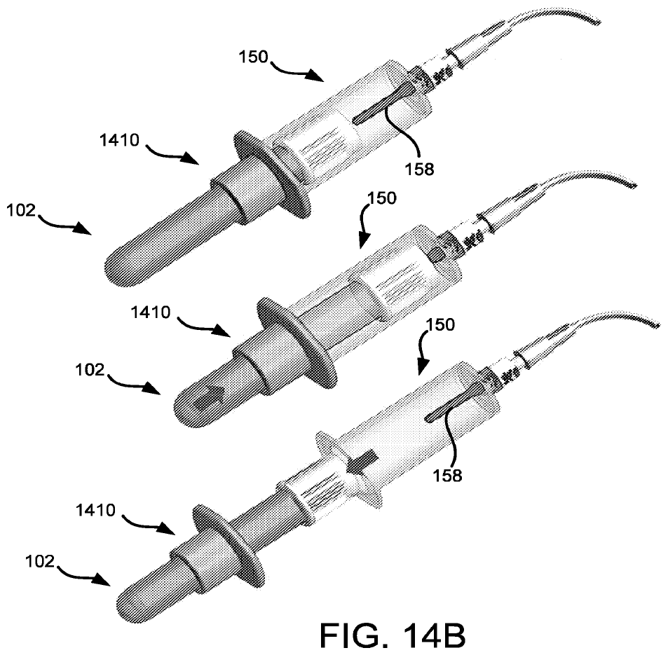

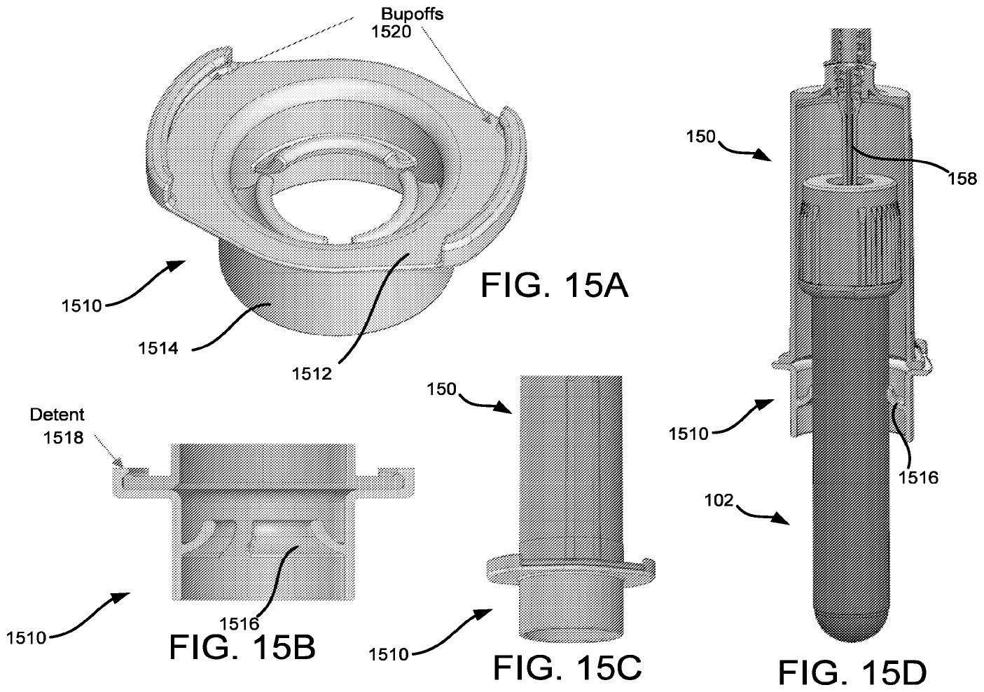

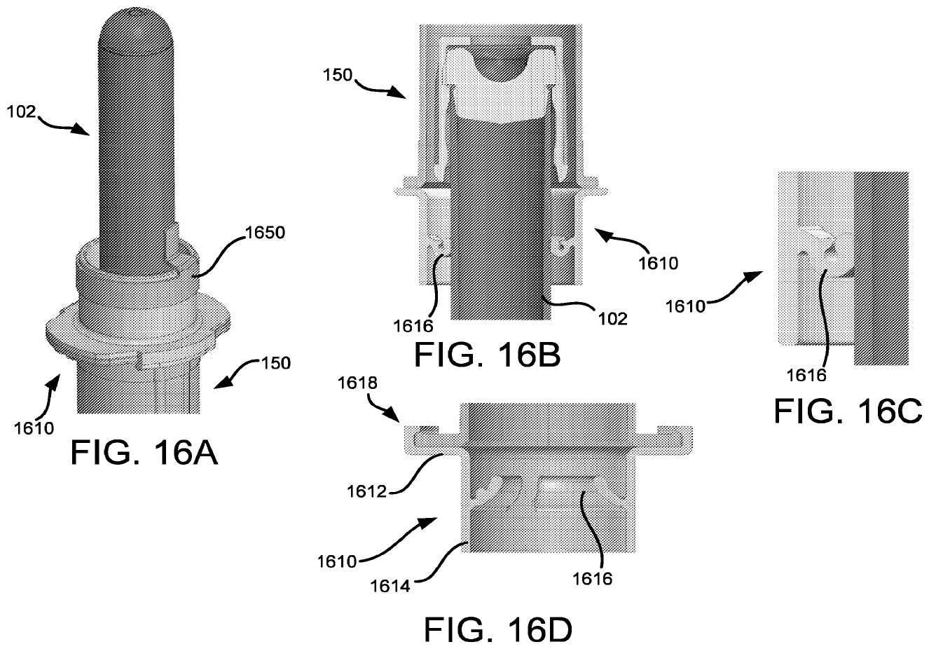

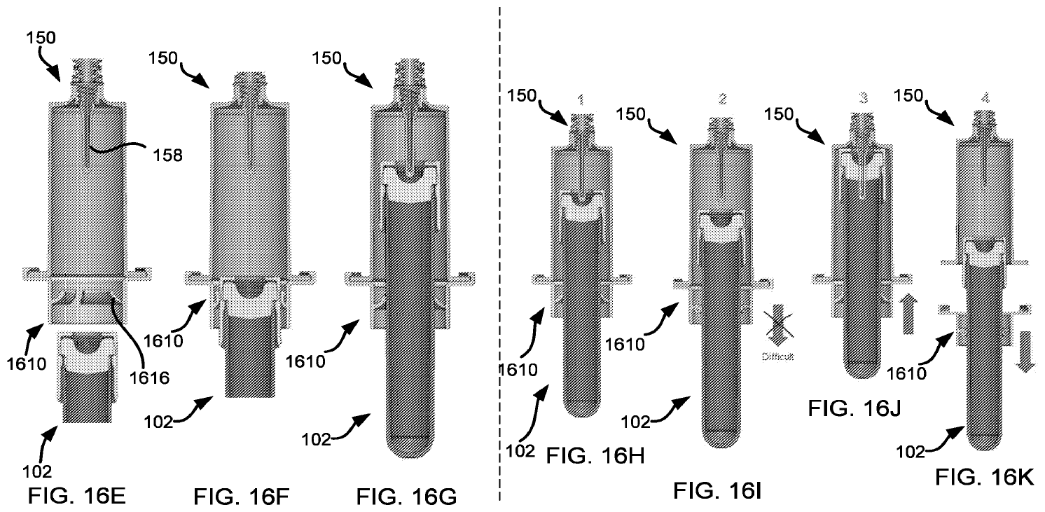

FIG. 13A-FIG. 13B is another aspect of an assembly with a securing mechanism 1310 that keeps the collection/discard tube 102 in spaced relation from the adaptor needle 158 during shipping and storage and will allow the collection/discard tube 102 to advance into engagement with the needle 158 in the adaptor 150 for sample collection. FIG. 13A-FIG. 13B illustrate the securing mechanism as a ratcheting collar 1310 with tabs 1312 that extend from a flanged portion 1314. The tabs 1312 are spaced around an inner circumference of the collar 1310 and extend distally from flanged portion 1314. The central opening of collar 1310 receives the tube 102 and the tabs 1312 provide an interference fit for the collar 1310 in the adaptor 150 and with the tube 102 such that the collection/discard tube may be advanced through the collar 1310. As shown in FIG. 13A, at least a portion of each of the tabs 1312 extends in a direction toward the center of the central opening of collar 1310. The flanged portion 1314 of the collar 1310 abuts against the open proximal end of adaptor 150 to hold adaptor 150 in place when tube 102 is pushed into adaptor 150. As illustrated in FIG. 13B, the collar 1310 remains in place as the tube 102 is advanced into engagement with the adaptor needle 158. The collar 1310 permits the collection/discard tube 102 to freely advance toward the needle 158, but tabs 1312 of the collar 1310 are configured to grip and bunch on the exterior of tube 102 when the collection/discard tube 102 is pulled away from the needle 158. The collar 1310 is therefore removed from the adaptor 150 when the collection/discard tube 102 is pulled from the adaptor 150. In one aspect, as shown in FIGS. 13A-13B, tabs 1312 may be supported by ring or rim 1312 that extends distally from flanged portion 1314. Alternatives to this configuration are described in FIG. 10A-FIG. 10B, FIG. 12, FIG. 14A-14D, FIG. 15A-15D, and FIG. 16A-16E. In this way, collar 1310 is like a one-way ratchet mechanism allowing easy advancement of the tube 102 into adaptor 150, but catching and gripping the tube 102 when the tube 102 is pulled out.

FIG. 14A-FIG. 14D is another aspect of an assembly with a securing mechanism 1410 that keeps the collection/discard tube 102 in spaced relation from the adaptor needle 158 during shipping and storage and will allow the collection/discard tube 102 to advance into engagement with the needle 158 in the adaptor 150 for sample collection. FIG. 14A-FIG. 14D is another alternative to FIG. 13A-FIG. 13B. FIG. 14A-FIG. 14D illustrate securing mechanism as a one-way collar 1410. The collar 1410 includes a tubular or barrel portion 1414 and a flanged portion. The collar 1410 includes internal grips 1416 disposed on and projecting from an inner surface of portion 1414. Grips 1416 provide a friction force for gripping the exterior of tube 102 when tube 102 is received into the interior of portion 1414. Grips 1416 are configured to hold to tube 102 in place (e.g., in a spaced relationship with needle 158) and permit the collection/discard tube 102 to be advanced into the adaptor 150 upon application of a pushing force onto tube 102. As illustrated in FIG. 14D, grips 1416 of the collar 1410 grip the tube 102 such that it is removed from engagement with the adaptor 150 when the collection/discard tube 102 is pulled from the adaptor 150. The flanged portion 1412 of collar 1410 has a catch 1418 that accepts the flange at the base or open proximal end of the adaptor 150. The catch 1418 may form all or part of the outer rim or circumference of the flanged portion 1412. The grips 1416 grip the exterior of tube 102, such that when tube 102 is pulled from the adapter 102, seal/coupling of the catch 1418 to the base of adapter 150 is broken. The catch 1418 may be made of a soft material (TPE) so that the seal/coupling with flanged base of the adaptor 150 can be broken and detached from the adaptor 150 when the tube 102 is pulled out of adaptor 150.

FIG. 15A-FIG. 15D illustrate an alternative securing mechanism 1510 to the collar 1310 of FIG. 13A-FIG. 13B and operates in a similar manner. The one-way collar 1510 in this aspect has portions 1512 and 1514. Portion 1512 includes a detent or catch 1518 that engages with the base of the adaptor 150 in a similar manner as described above in relation to catch 1418. As such, in this aspect, the collar 1510 does not have an interference fit with the adaptor 150. Because of bump-offs 1520 in the detent 1518 on the collar 1510, the collar 1510 cannot advance upward into the adaptor 150. Collar 1510 includes grips 1516, which extend upward (distally) and toward the center of the interior of portion 1514 from the interior wall of portion 1514. The grips 1516 act as a one-way ratcheting mechanism on tube 102. In use, the collar 1510 is disposed around tube 102 and grips 1516 hold to the exterior of tube 102 and detent 1518 engages with the base of the adapter 1510 to hold the tube 102 in a spaced relationship with needle 158. The grips 1516 are configured to permit easy movement of the tube 102 toward the interior of adaptor 150 and into engagement with needle 158 with relatively low pushing force. Moreover, grips 1516 are configured to bunch and provide high resistance or friction force on the exterior of tube 102 when tube 102 is pulled toward the exterior of adaptor 150. An example of the operation and bunching that occurs for grips 1516 is shown for the similarly configured grips 1616 in FIGS. 16B, 16C, and 16E-16K. The grips 1516 hold to the collection/discard tube 102 such that detent 1518 is detected from the base of the adapter when tube 102 is removed or pulled from the adaptor 150. Thus, the collar 1510 and the tube 102 are both removed from adapter 150 by the pulling of tube 102.

FIG. 16A-FIG. 16D illustrate an alternative securing mechanism 1610 to the collar of FIG. 15 with a sticker 1650 added to secure the collar 1610 to the collection/discard tube 102 in the shipping/storage position. The collar 1610 includes portions 1612, 1614, which are configured with similar features as portions 1512, 1514 described above. The bump-offs and detents 1618 are rotated ninety degrees relative to what is illustrated in FIG. 15A-FIG. 15D.

FIG. 16E-FIG. 16K illustrate the operation of the assembly of FIG. 16A-FIG. 16D. The upward facing grips 1618 make it feasible to advance the collection/discard tub 102 upwards into the adaptor 150 but, when the collection/discard tube 102 is advanced out of the adaptor 150, it carries the collar 1610 with the tube 102 from the adaptor 150. Grips 1616 act as a one-way grip so that the tube 102 may be advanced upward through the grips, but the tube 102 carries the collar 1610 when advanced outward from the adapter.

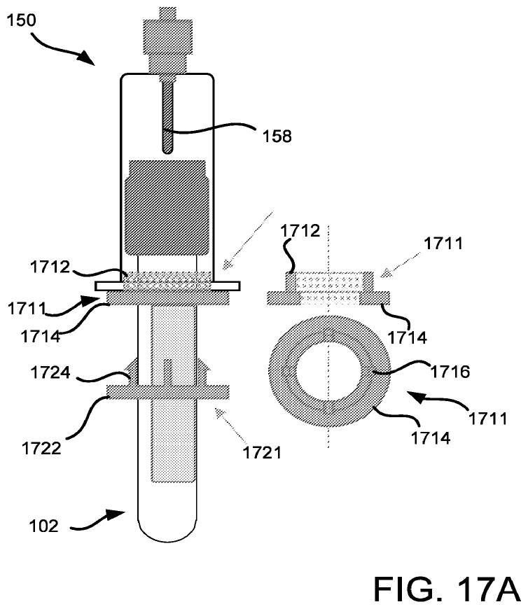

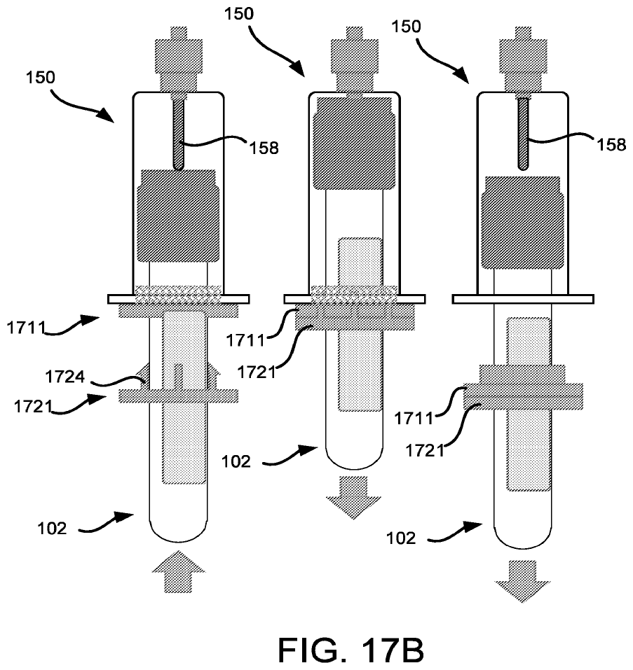

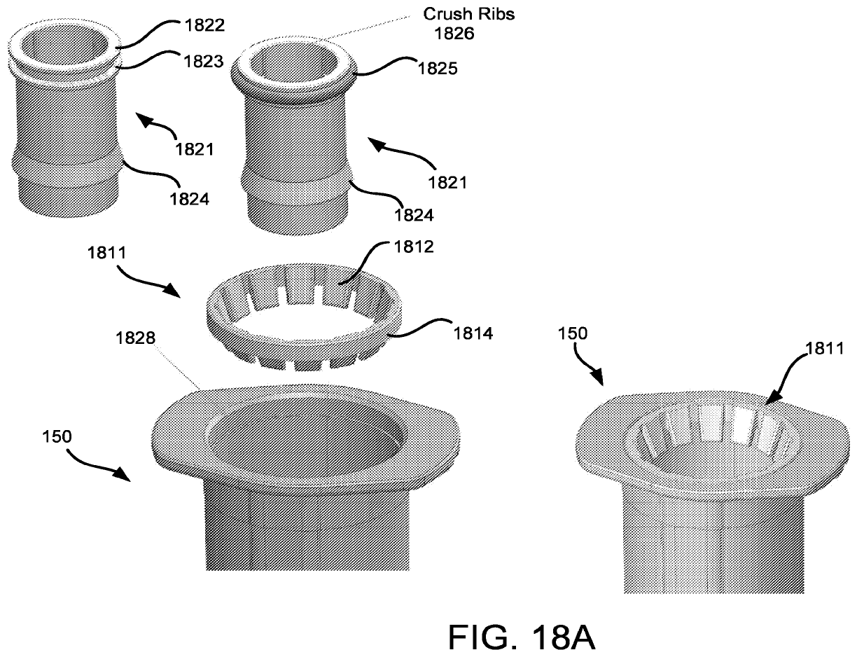

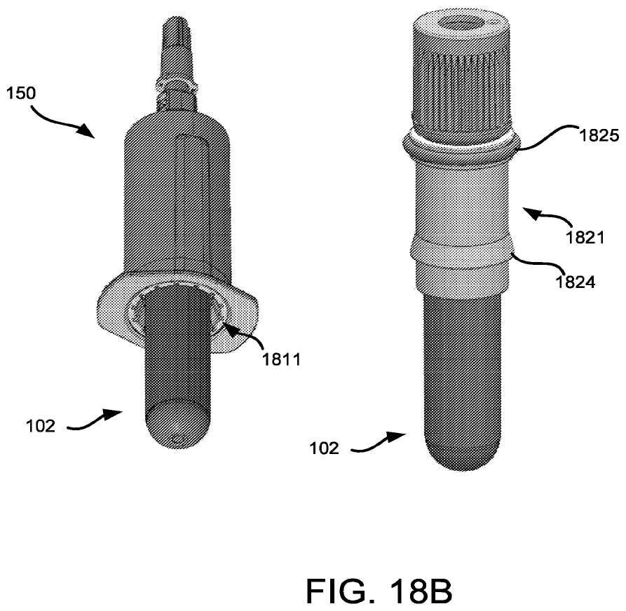

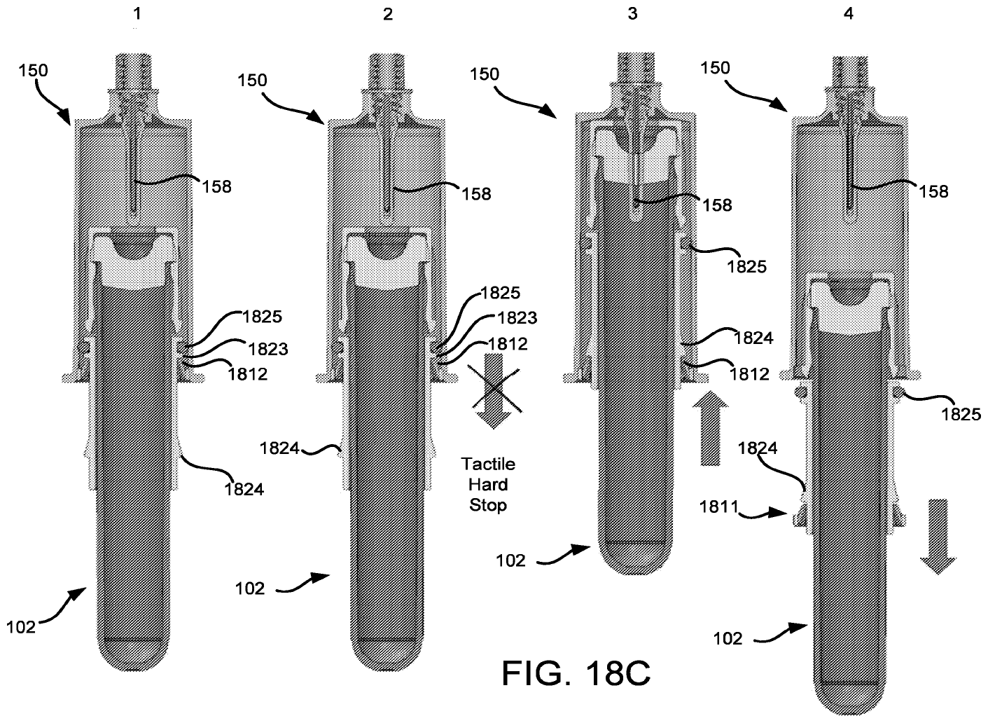

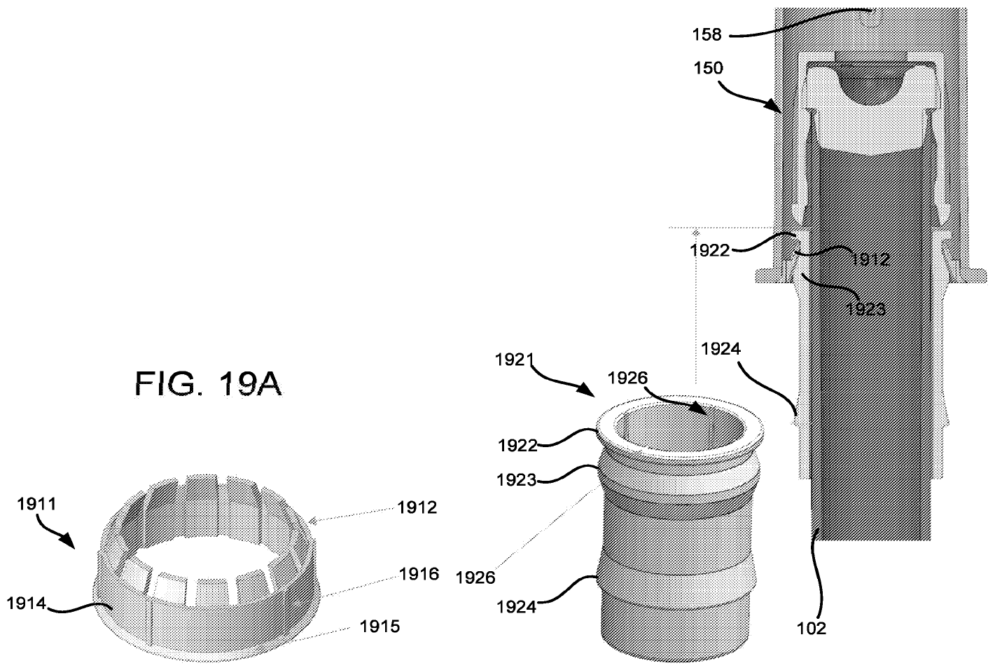

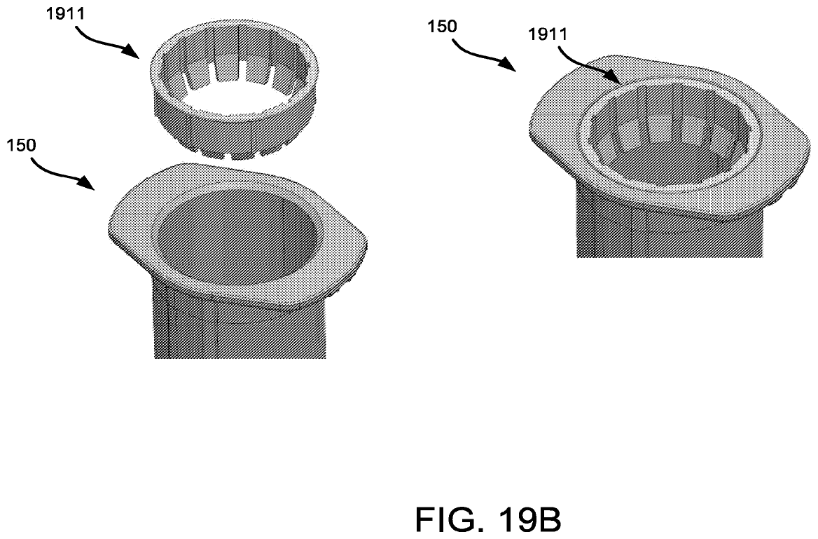

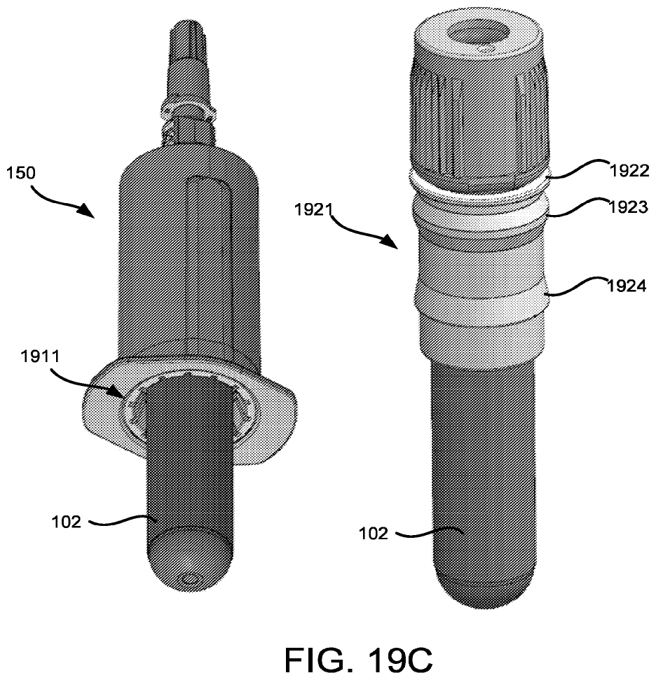

FIG. 17A-FIG. 17B is a two-piece securing mechanism. The securing mechanism 1710 illustrated in FIGS. 17A-17B includes collars 1711 and 1721. Variations of this aspect are illustrated in FIG. 18A-FIG. 18C and FIG. 19A-FIG. 19C. Collar 1721 includes a base portion 1722 and barbs 1724 that extend longitudinally (i.e., in the direction of insertion and extraction of the tube 102 into adaptor 150) from base portion 1722. The collar 1722 is affixed (e.g., adhered) to the exterior of the collection/discard tube 102 by an interference fit. Collar 1711 includes portions 1712, 1714, which are configured in a similar manner to portions 512, 514 described above to form an interference fit with the open base end of adaptor 150 and to prevent collar 1711 from being advanced further into adaptor 150. The inner diameter of collar 1711 provides an interference grip onto the exterior of tube 102 to hold tube 102 in a stationary position relative to collar 1711 and adaptor 150 unless a sufficient force is applied to tube 102. Collar 1711 includes holes 1716 configured to receives barbs 1724. Tube 102 is held by collar 1711 in a spaced relationship with needle 158 during shipment and storage with the collar 1721 in a spaced relationship with collar 1711. When tube 102 is pushed or advanced into the adaptor 105 and into engagement with needle 158, the collar 1721 is advanced toward collar 1711 and the barbs 1724 lock into holes 1716 in a flanged adaptor collar 1711 which is in an interference fit with the adaptor base. The barbs 1724 of collar 1722 lock into openings 1716 in the flanged adaptor collar 1711, forming a two-piece assembled collar. When the collection/discard tube 102 is advanced out of the adaptor 150, tube 102 carries the two-piece assembled collar (comprising collars 1711, 1721) from the adaptor 150.

FIG. 18A-FIG. 18C is another securing mechanism configured as a two-piece assembly for holding the collection/discard tube 102 in spaced arrangement from the needle 158 in the adaptor 150 during shipment but allowing the collection/discard tube 102 to be advanced into engagement with the needle 158 for sample collection. The two-piece assembly or securing mechanism is pulled from the adaptor 150 when the collection/discard tube 102 is removed therefrom. The securing mechanism comprises a snap ring 1811 with a ring portion 1814 and with upward facing projections or snap fins 1812 that project from ring portion 1814 and are biased to be angled toward the interior of snap ring 1811. The snap ring 1811 is fitted into the open end base of the adaptor 150. The adapter 150 includes a stepped end 1828 to receive the snap ring 1811. The securing mechanism further comprises a sleeve 1821 and raised portions 1822 and 1823 configured to retain an O-ring 1825 between portions 1822 and 1823. Sleeve 1821 further includes raised portion or flange 1824. The sleeve 1821 is fitted on the collection discard tube 102 (e.g., sleeve 1821 may be adhered to the exterior and/or tube 102 or by grip the exterior of tube 102 with crush ribs 1826). When the tube 102 is inserted into adaptor 150, the projections 1812 permit the raised portions 1822, 1823 and O-ring 1825 to pass through the snap ring 1811. The O-ring 1825 provides friction against the interior of adaptor 150 to hold tube 102 in a spaced relationship with needed 158 during shipment and/or storage. The projections 1812 of the snap ring 1811 sign against the exterior of sleeve 1821 and abut against the raised portion 1823 and thus do not permit the tube 102 to be pulled out of the adaptor 150. The tube 102 is pushed into adaptor 150 to engage needed 158 for sample collection. During this engagement, the projections 1812 permit the raised portion 1824 to pass through the snap ring 1811. When the tube 102 is pulled out of the adaptor 150, the flange or raised portion 1824 on the sleeve 1821 catches on the ends of projections 1812 and pulls the snap ring 1821 from the adaptor 150 when the collection/discard tube 102 is removed therefrom.

FIG. 19A-FIG. 19C is an alternative aspect to the two-piece assembly in FIG. 18A-FIG. 18C for holding the collection/discard tube 102 in spaced arrangement from the needle 158 in the adaptor 150 during shipment but allowing the collection/discard tube 102 to be advanced into engagement with the needle 158 for sample collection. The two-piece assembly or securing mechanism is pulled from the adaptor 150 when the collection/discard tube 102 is removed therefrom. The securing mechanism comprises a snap ring 1911 and a sleeve 1921. The snap ring 1921 comprises upward facing projections or snap fins 1912 that project for a ring portion or base 1914 and are biased to be angled toward the interior of snap ring 1911. The snap ring form an interference fit on the collection/discard tube 102. The snap ring 1911 further comprises ribs 1916 for gripping the interior of adaptor 150 and a flange 1915 formed in the base 1914 for abutting against the rim of the open base end of adaptor 150. The snap ring 1911 is fitted into the base of the adaptor 150 as shown with the ribs 1916 gripping the interior of adaptor 150 and the flange 1915 preventing the snap ring 1911 from being advanced further into the adaptor 150. In this aspect the sleeve 1921 has first, second, and third flanges or raised portions 1921, 1922, 1924 projecting from the exterior of the sleeve 1921. The sleeve 1921 further comprises ribs 1926 projecting from the interior surface of sleeve 1921 for gripping the exterior of tube 102 when sleeve 1921 is fitted on the collection/discard tube 102 such that sleeve 1921 and tube 102 are stationary relative to each other. When the tube 102 is advanced into the adaptor 150 for shipment, the fins 1912 permit the first raised portion 1922 to advance past the fins 1912 such that the ends of the fins 1912 are inserted between raised portions 1922 and 1923 to lock the tube 102 and sleeve 1921 in place for shipment and storage with the tube 102 in a spaced relationship with the needle 158. The inner surfaces of the fins 1912 lay against a portion of the second flange or raised portion 1923 in this position, holding the collection/discard tube 102 in place for shipment or storage. The ramped flange 1923 advance past the snap ring 1911 when the collection/discard tube in place in the second position. The fins 1912 permit the second and third raised portions 1923 and 1924 to pass through snap ring 1911 when the tube 102 is pushed into adaptor 150 to such that the collection tube 102 is engaged with the needle 158 for sample collection. When the tube 102 is pulled out of the adaptor 150, the third flange or raised portion 1924 on the sleeve 1921 catches on the ends of projections 1912 and pulls the snap ring 1911 from the adaptor 150 when the collection/discard tube 102 is removed therefrom.

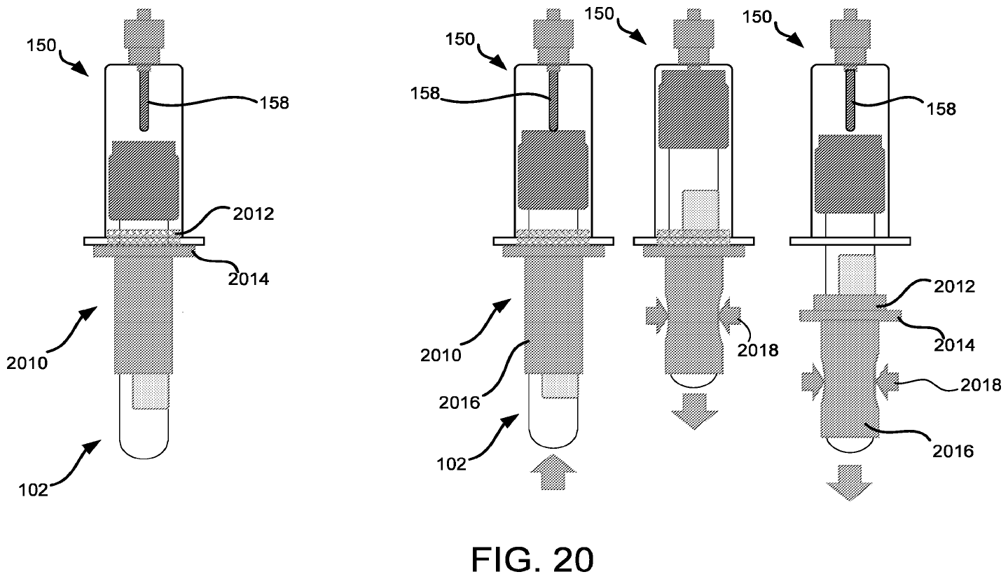

FIG. 20 is a collection/discard tube 102 with securing mechanism 2010 including a flanged sleeve disposed around the tube 102 for holding the collection/discard tube 102 in spaced arrangement from the needle 158 in the adaptor 150 during shipment but allowing the collection/discard tube 102 to be advanced into engagement with the needle 158 for sample collection. The securing mechanism 2010 includes a sleeve 2016 coupled to and extending from a flanged portion comprising portions 2012, 2014. Portions 2012, 2014 may be configured with the same features described above in relation to portions 112, 114 of securing mechanism 110 and portions 512, 514 of securing mechanism 510. In this way, portions 2012, 2014 of securing mechanism 2010 provide an interference fit between the securing mechanism 2010 and the adaptor 150. The flanged portion 2014 is engaged with the adaptor 150 through an interference fit. There is clearance between the outer diameter of the collection/discard tube 102 and the inner diameter of the sleeve portion 2016 that, upon application of a pushing/pulling force onto tube 102 permits easy (or free) movement of the collection/discard tube 102 in the securing mechanism 2010 and into the adaptor 150. Portion 2014 has a larger diameter than the proximal end of adaptor 150 and prevents the securing mechanism 2010 from advancing into adaptor 150 beyond the open end of the adaptor 150 when the collection/discard tube 102 is placed therein. The flanged sleeve portion 2016 is flexible. When the collection/discard tube 102 is advanced through the securing mechanism 2010 and into engagement with the needle 158 in the adaptor 150 for blood collection, the securing mechanism 2010 remains in position. There is diametric clearance between the tube and the sleeve 2016 that allows free movement of the tube in the sleeve 2016. The operator grips the collar adjacent the bottom of the adaptor 150 when advancing the collection/discard tube 102 into engagement with the needle 158 in the adaptor 150. Since the sleeve 2016 is flexible, as indicated by arrows 2018, the sleeve 2016 can assist with gripping (e.g., when gripped by an operator) the collection/discard tube 102 when removed from the adaptor 150. Portion 2016 is dimensioned with a sufficient length such that when the tube 102 is advanced into adaptor 150 and engaged with needle 150, the only way to grab the tube 102 is by grabbing the sleeve 2016 (as indicated by arrows 2018). This forces the user to pull the securing mechanism 2010 out with the tube 102. When the collection/discard tube 102 is pulled from the adapter 150, the interference fit between portion 2012 and adaptor 150 is overcome and the flanged sleeve securing mechanism 2010 (which is being gripped by the user) is removed along with the collection/discard tube 102.

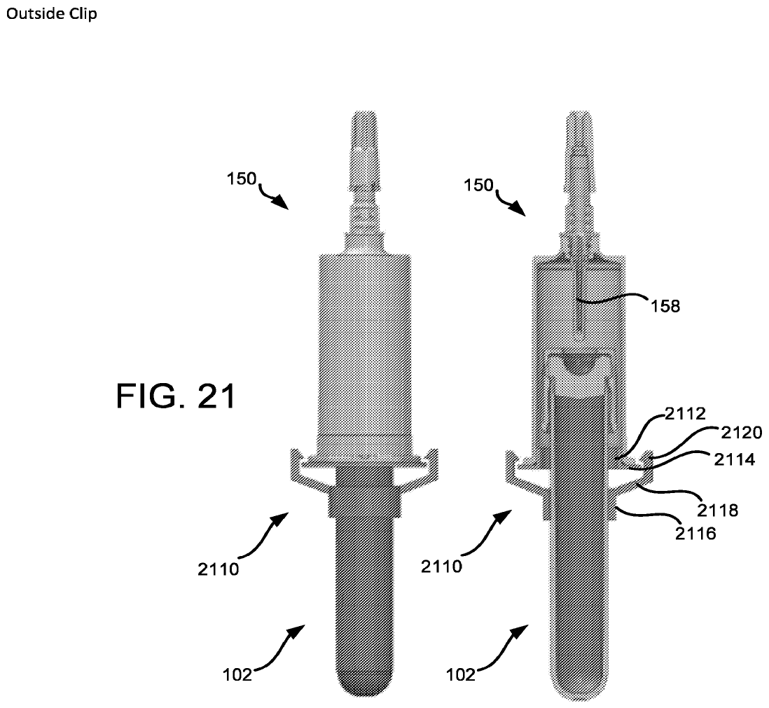

FIG. 21 illustrates a securing mechanism 2110 including a two-part collar with a collar including portions 2112, 2114 that fits in the bottom portion of the adaptor 150 but does not advance therein, e.g., in the same manner described in relation to the collars in FIGS. 5A-5D and FIG. 20 above. The first portion of the collar 2112, 2114 in the base of the adaptor 150 cooperates with the collar on the collection/discard tube 102 to hold the collection/discard tube 102 in spaced relation to the needle 158 in the adaptor 102. The second portion 2116 of the collar on the collection/discard tube 102 has arms 2108 with notched engagement members 2120 that will advance past the first portion 2114 of the collar as the collection/discard tube 102 is advanced into the adaptor 150. As such, the second portion 2116 of the collar is fashioned as an outside clip. When the collection/discard tube 102 is removed from the adaptor 150 after sample collection, the engagement members 2120 on the second portion 2118 of the collar pull the first portion 2114 of the collar from the base of the adaptor 150.

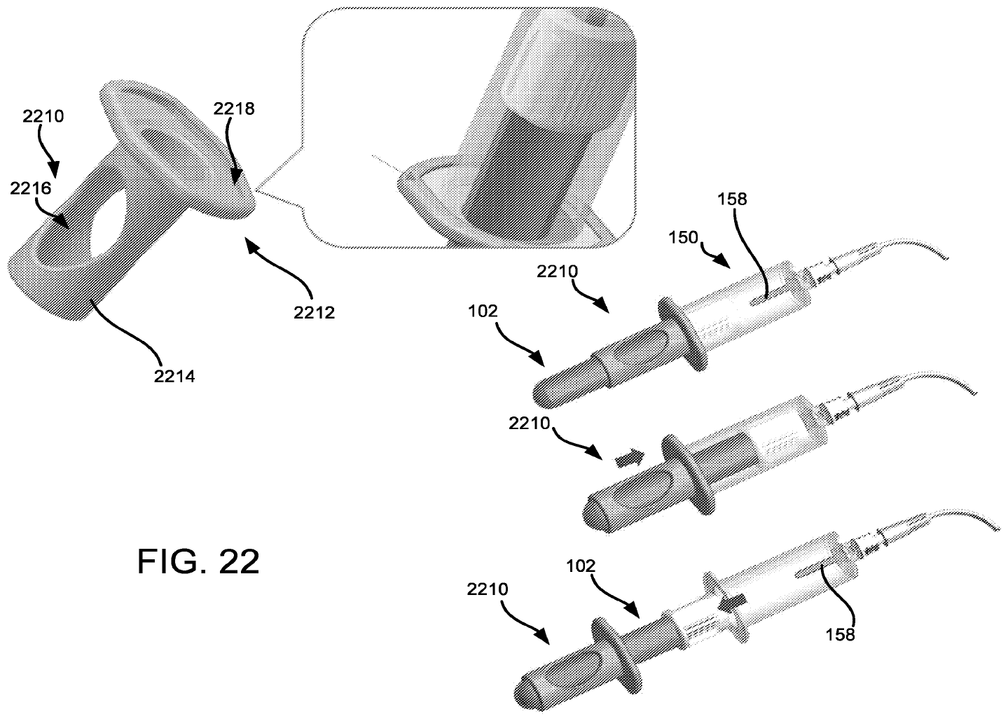

FIG. 22 is another securing mechanism 2210 configured as a flanged sleeve for the collection/discard tube 102 that is an alternative to what is illustrated in FIG. 20. Securing mechanism 2210 includes flanged portion 2212 and sleeve portion 2214. The flange 2212 locks to the flange at the base of the adaptor 150 with a lip/detent 2218 that fits over the adaptor base flange in the same manner described above in relation to flanged portion 4112 including catch 1418 in FIGS. 14A-14D. As such, the securing mechanism 2210 is secured by a lock fit with adaptor 150 instead of an interference fit. The inner diameter of securing mechanism 2210 grips the outer diameter of tube 102 and collection/discard tube 102 is held in spaced arrangement from the needle 158 in the adaptor 150 during shipment by the securing mechanism 2210. Advancing the collection/discard tube 102 through the securing mechanism 2210 advances the collection/discard tube 102 into connection with the adaptor needle 158. After the sample is collected, the flanged portion 2212, which is flexible, is detached from the flange at the base of the adaptor 150 and the collection/discard tube 102, with the sleeve 2210 attached, is removed from the adaptor 150. In one aspect, the flanged portion 2212, when locked to the base of the adaptor 150 creates a sealed unit with the adaptor 150 for increased sterilization. In one aspect, the sleeved portion includes one or more opening(s) 2216 to enable the operator to view the contents (e.g., the sample collected) within tube 102.

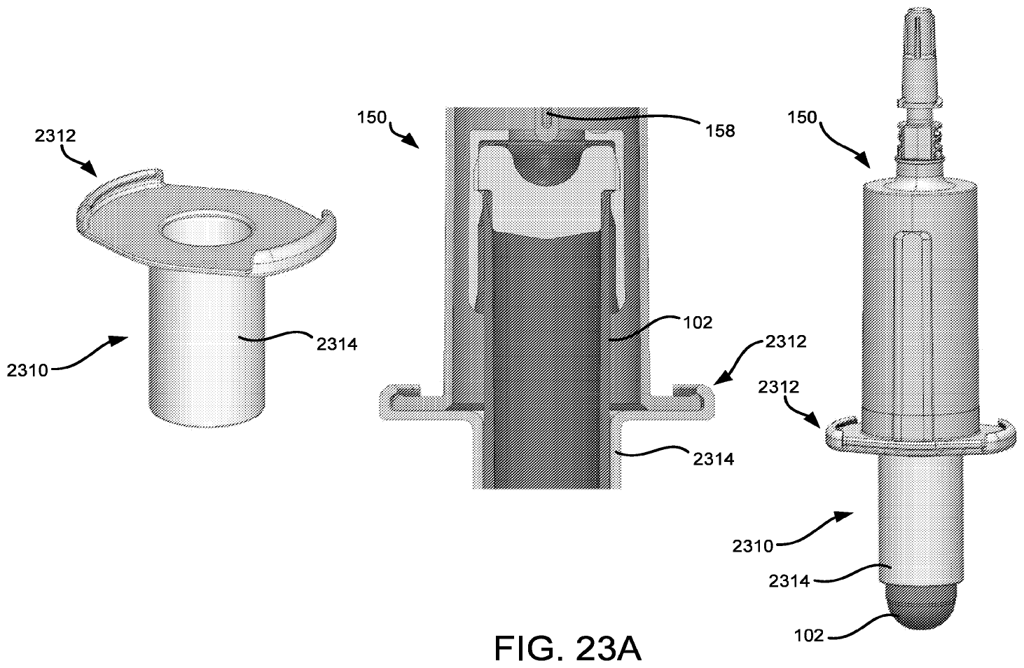

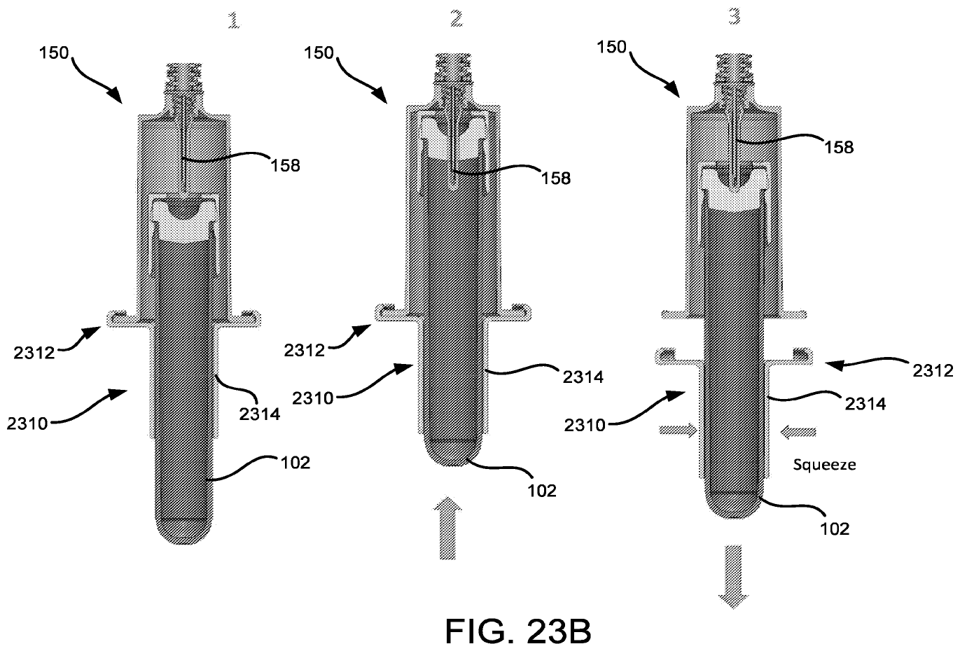

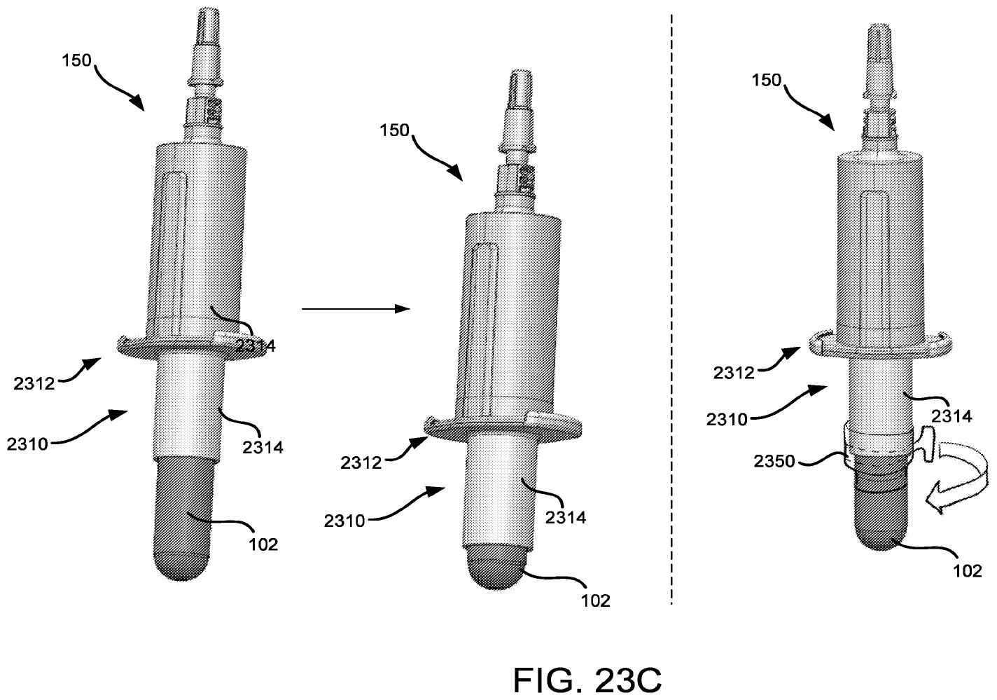

FIG. 23A-FIG. 23C is another securing mechanism 2310 configured as a flanged sleeve that is an alternative to what is illustrated in FIGS. 20 and 22. The securing mechanism 2310 in this aspect includes portions flanged portion 2312 and sleeve portion 2314. Flanged portion 2312 has a detent with bump-off for locking onto the base of the adaptor 150 as previously described and illustrated. The flanged sleeve 2310 is detached from the adaptor 150 by squeezing the (elastomeric) sleeve 2314 and applying a pulling force away from the adaptor 150, which causes the sleeve flange 2312 to separate from the flange at the base of the adaptor 150 when the collection/discard tube 102 is removed from the adaptor 150. The sleeve 2314 provides a gripping surface for removing the collection/discard tube 102. There is diametric clearance between the sleeve 2314 and the collection/discard tube 102. In one aspect, some or all of the sleeve portion 2314 may be made of a clear elastomeric material such that the contents of tube 102 are visible through the sleeve portion 2314. In one aspect, the sleeve portion 2314 is affixed to the collection/discard tube 102 by shrink wrap or tape 2350 (shown in FIG. 23C) during shipment. The shrink wrap or tape 2350 (in addition to the friction provided by the interior of the sleeve portion 2314) helps ensure that the collection/discard tube 102 remains spaced apart from the needle 150 during shipment.

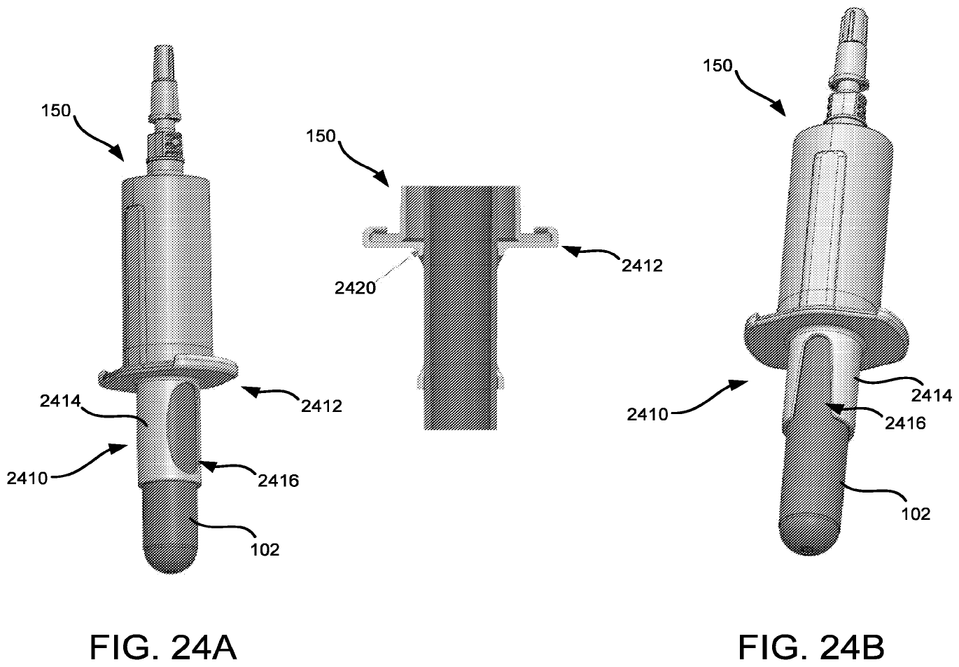

FIG. 24A-FIG. 24B is another securing mechanism 2410 and another aspect of what is illustrated in FIG. 20 and FIG. 23A-FIG. 23C. Securing mechanism 2410 includes flanged portion 2412 and sleeve portion 2410, which includes similar features to the fanged portions 2212, 2312 and sleeve portions 2214, 2314, described above. Sleeve portion 2414 includes opening(s) 2416 for enabling the user to view the contents of tube 102. Each of the opening(s) 2416 may be formed as a closed loop (FIG. 24A) or a slot (FIG. 24B). In one aspect, the securing mechanism 2410 may include friction bead 2420 on the interior circumference of securing mechanism 2410 (e.g., inner circumference of portion 2412) to add additional friction and aid in retaining tube 102 in a stationary position relative to securing mechanism 2410. FIGS. 24A and 24B show how the sleeve flange securing mechanism 2410 seats on the flange at the base of the adaptor 150 to retain the collection/discard tube 102 in the spaced position from the adaptor needle 150 during shipment. As described above, the tube 102 may be moved in relation to securing mechanism 2410 and adaptor 150 upon application of a pushing or pulling force into/out of the adaptor 150.

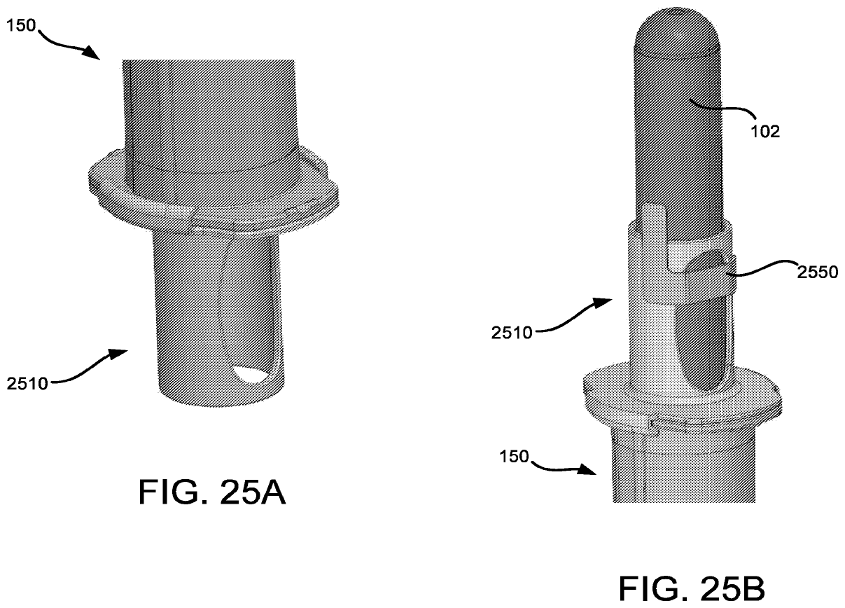

FIG. 25A-FIG. 25C is another securing mechanism 2510 and another aspect of what is illustrated in FIG. 20, FIG. 23A-FIG. 23C, and FIGS. 24A-24B. Securing mechanism 2510 includes the same features (i.e., flanged portion and sleeve portion with opening(s)) as securing mechanism 2410. In this aspect, as shown in FIG. 25B, the securing mechanism 2510 may include a tape or adhesive strip 2550 (e.g., U-shaped, L-shaped, etc.) that is affixed over a portion of the securing mechanisms 2510 (e.g., over the sleeve portion) and over a portion of tube 102 to aid in retaining tube 102 in a stationary position relative to securing mechanism 2510 (and adaptor 150). The strip 2550 may be positioned and configured to contact the tube 102 through one of the openings in the securing mechanism 2510. The securing mechanism 2510 holds the tube 102 in a spaced relationship with needle 158 during shipment and allows the tube 102 to be moved relative to securing mechanism and adaptor 150 upon application of a pulling or pushing force into/out of adaptor 150. It is to be appreciated that, after shipment and during use, the pulling and pushing forces on tube 102 will decouple the strip 2550 from tube 102.

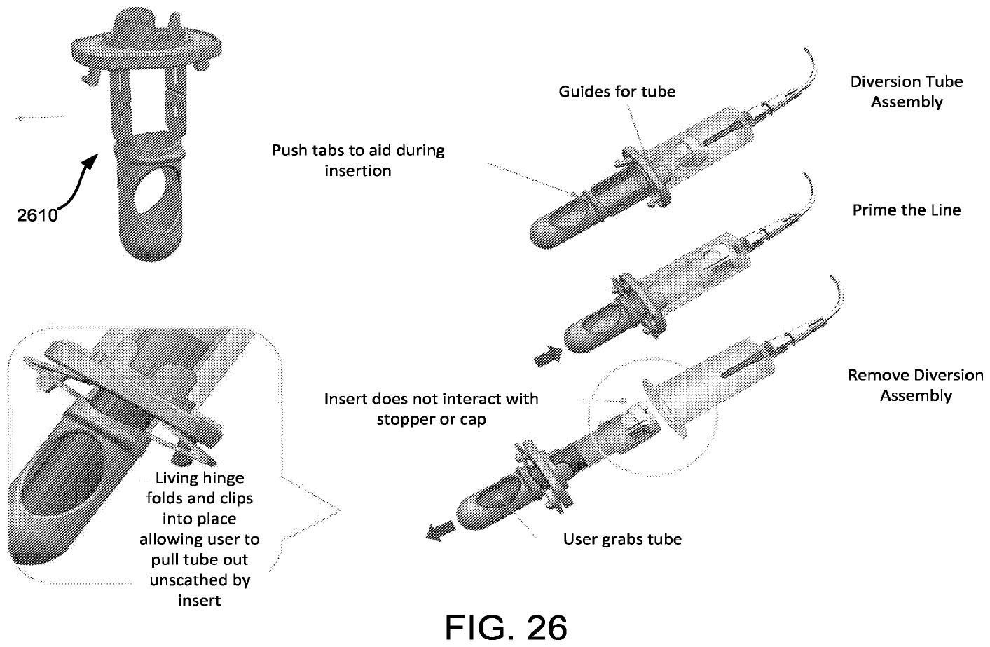

FIG. 26 illustrates a securing mechanism 2610 configured as a collar for the collection/discard tube 102 that has a collapsible section with a living hinge that allows the collar to keep the collection/discard tube in spaced relation to the needle in the adaptor during shipment but, for blood collection, as the collection/discard tube is advanced toward the needle, the living hinge collapses, allowing the collection/discard tube to be advanced into contact with the adaptor needle for blood collection. After collection, the collar is removed with the collection/discard tube as it is removed from the adaptor.

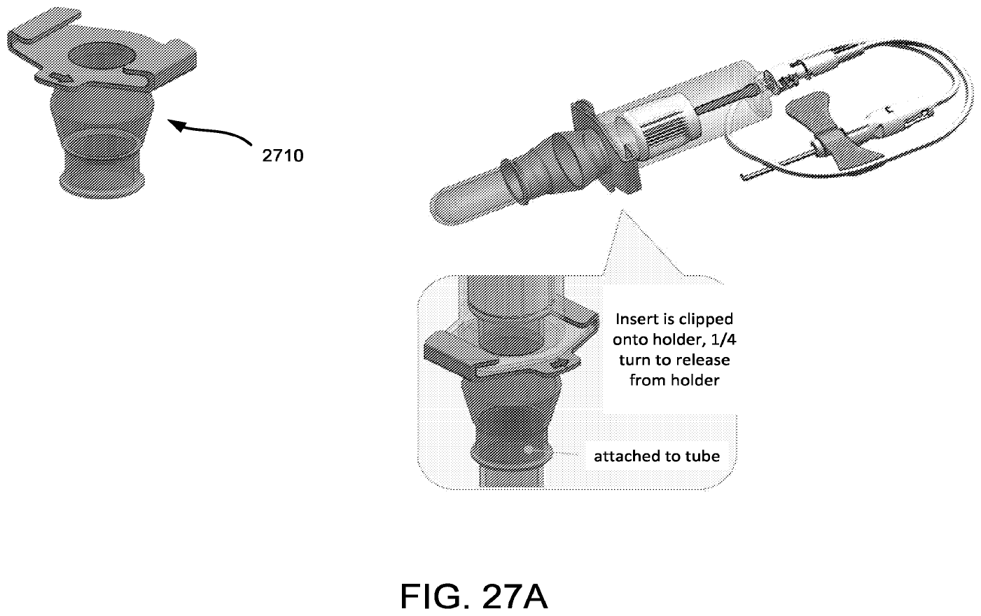

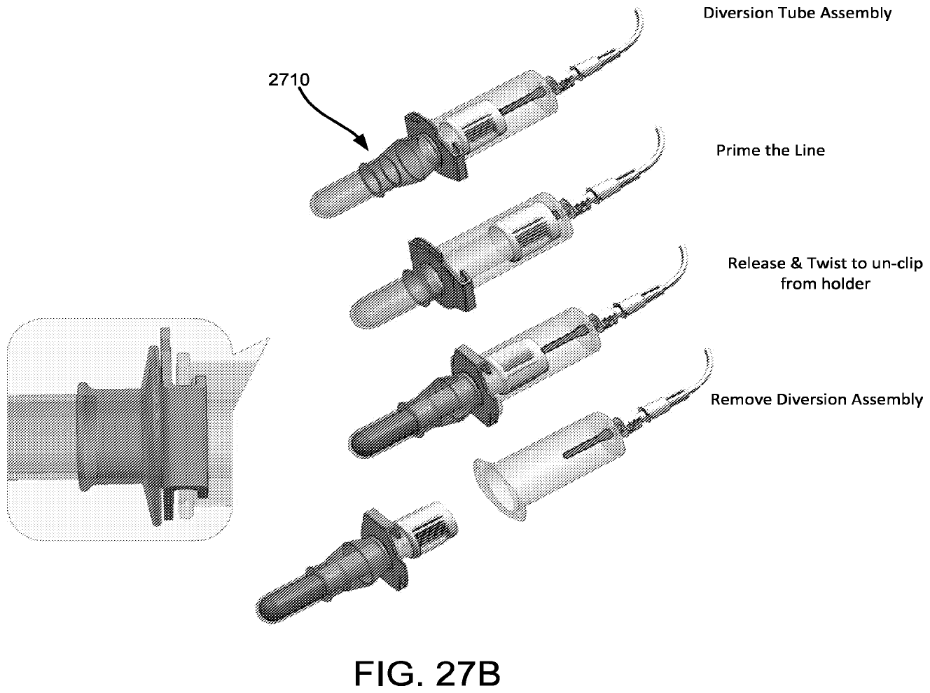

FIG. 27A-FIG. 27B is another securing mechanism 2710 and another aspect of the collection/discard tube assembly wherein the collection/discard tube has a sleeve affixed thereto with a twist-on, twist-off flange that attaches to the flange at the base of the adaptor. As illustrated, the line set is in fluid communication with the adaptor and blood is drawn into the collection/discard tube when the needle in the adaptor pierces the seal in the cap of the collection/discard tube when it is advanced into contact with the needle. The collection/discard tube is advanced in the sleeve, which remains fixed when locked on the flange of the adaptor. When the sleeve is twisted off the adaptor flange, the collection/discard tube is removed from the adaptor.

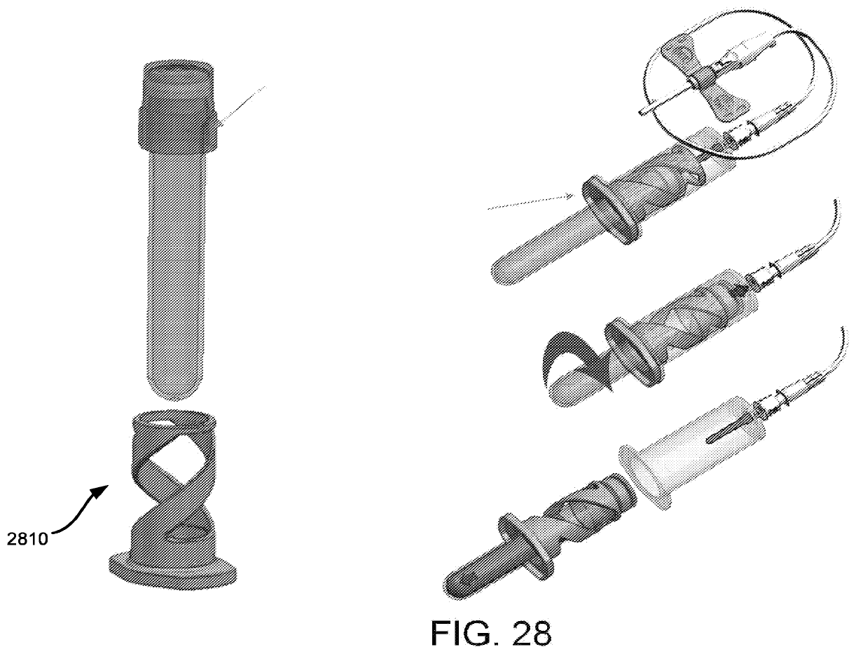

FIG. 28 is another securing mechanism 2810 and another aspect of the collection/discard tube assembly for holding the collection/discard tube in spaced relation from the needle in the adaptor during shipment but allowing the collection/discard tube to be advanced into engagement with the needle for sample collection. In this aspect, the collection/discard tube has a sleeve that can be threaded into the adaptor. There is a flange at the base of the sleeve, which, as illustrated, has helical spines that provide a friction fit when advance into the adaptor. The flange limits how far the sleeve may advance. The sleeve holds the collection/discard tube in spaced relation from the needle during shipment and storage. The collection/discard tube is twisted to advance it within the sleeve. As illustrated, the line set is in fluid communication with the adaptor and blood is drawn into the collection/discard tube when the needle in the adaptor pierces the seal in the cap of the collection/discard tube when it is advanced into contact with the needle. After sample collection, the collection/discard tube is removed from the adaptor.

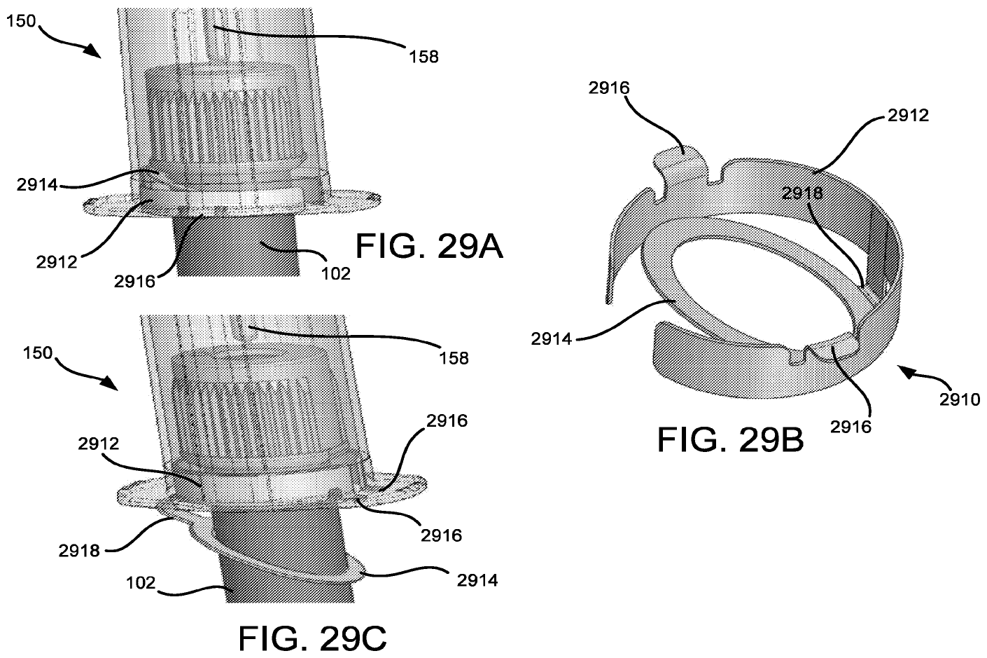

FIGS. 29A-29C illustrate another securing mechanism 2910 and another aspect of the collection/discard tube assembly for holding the collection/discard tube 102 in spaced relation from the needle 158 in the adaptor 150 during shipment but allowing the collection/discard tube 102 to be advanced into engagement with the needle 158 for sample collection. The securing mechanism 2910 has a collar 2912 and a flexible hinged spring ring 2914 attached to collar 2912 by hinge 2918. The ring 2918 is biased by hinge 2918 to a slanted or angled position relative to collar 2912. In one aspect, as shown in FIG. 29A, the ring 2918 is biased to be slanted toward the interior of collar 2912 and toward the interior of adaptor 150. In another embodiment, as shown in FIG. 29C, the ring 2918 is biased to be slanted toward the exterior of collar 2912 and exterior of adaptor 150.

The collar 2912 includes an outer diameter selected to fit (e.g., snugly) within the inner circumference at the open end of the base of the adaptor 150 in an interference fit. The securing mechanism 2910 includes one or more tabs 2916 which extend away from the outer circumference of collar 2912. Tabs 2916 are configured to abut against the circumference defining the opening at the end of the base of adaptor 150 to prevent the securing mechanism 2910 from being advanced further into the interior of adaptor 150 and to hold the securing mechanism 2910 in position at the base of adaptor 150. The flexible hinged spring ring 2912 is configured to receive the tube 102 and to grip and hold the exterior of the collection/discard tube 102 in spaced relation from the needle 158 during shipment and storage. When the ring 2914 is at an angle relative to the base of the adaptor and/or the collar 2912, the inner circumference of ring 2914 makes contact with the collection/discard tube and grips or locks against the exterior of tube 102. The grip is such that, upon application of pulling or pushing force onto the tube 102, the tube 102 is moveable in relation to the securing mechanism 2910 and adaptor 150 into and out of the adaptor 150. The ring 2914 bends in the direction of the tube movement. When the tube 102 causes the ring 2914 to move toward the adaptor 150, the ring 2914 reduces or releases contact with the collection/discard tube 102 and allows the collection/discard tube 102 to advance. Blood is drawn into the collection/discard tube 102 when the needle 158 in the adaptor 150 pierces the seal in the cap of the collection/discard tube 102 when it is advanced into contact with the needle 158. After sample collection, the collection/discard tube 102 is removed from the adaptor 150 along with the securing mechanism 2910. When the collection/discard tube 102 is pulled away from the needle 158 in the adaptor 150, the ring 2914 bends away from the holder and grasps or pinches the collection/discard tube 102 more tightly. This locks the ring 2914 against the collection/discard tube 102 and the collection/discard tube 102 pulls the securing mechanism 2910 with it when the collection/discard tube 102 is removed from the adaptor 150.

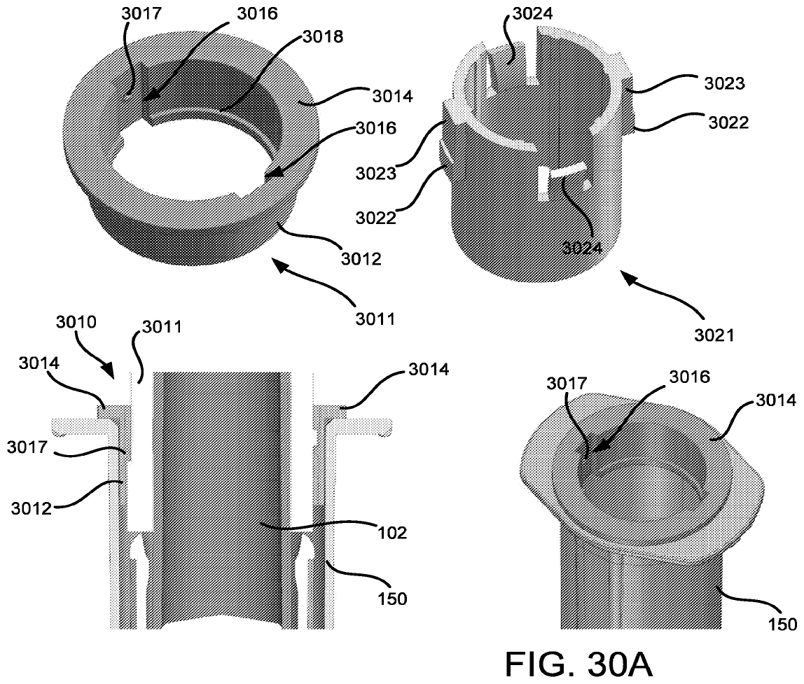

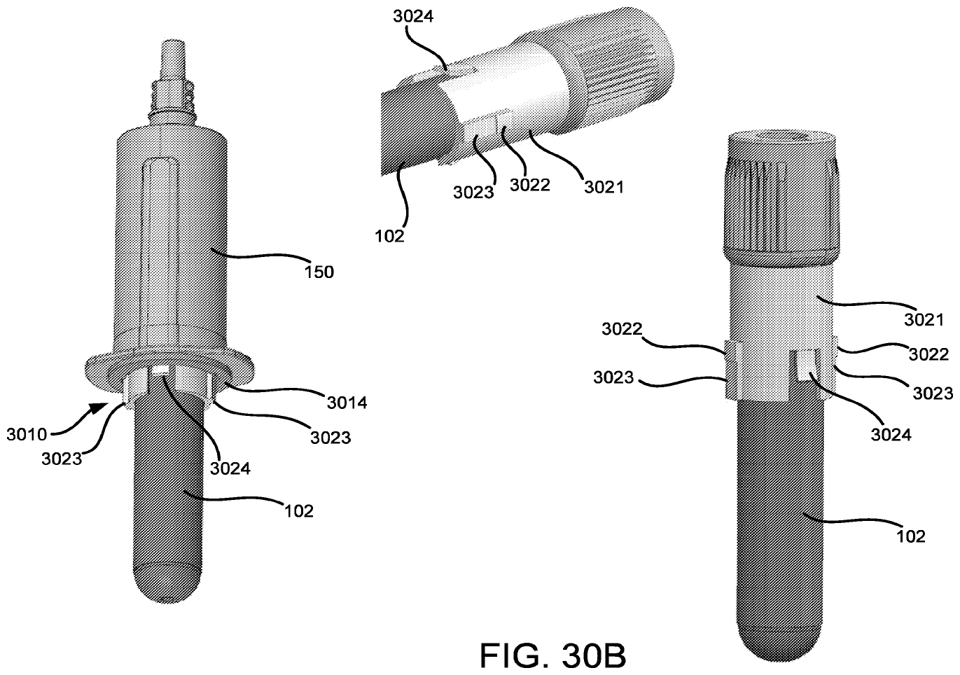

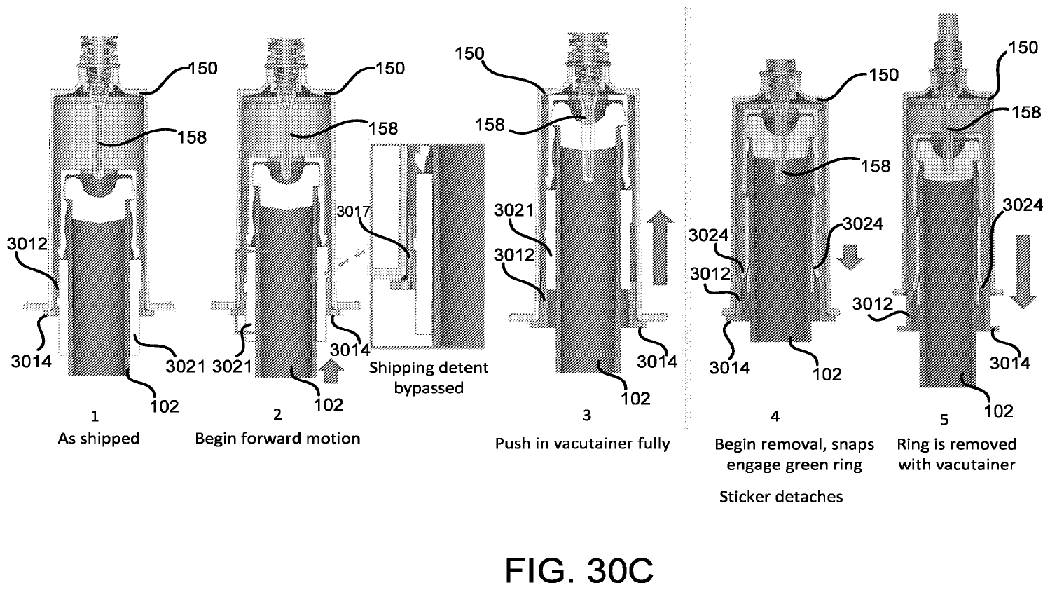

FIG. 30A-FIG. 30C illustrate another securing mechanism 3010 and another aspect of a two-piece collar/sleeve assembly for holding the collection/discard tube 102 in spaced relation to the needle 158 in the adaptor 150 during shipment and storage but allowing the collection/discard tube 102 to be advanced into engagement with the needle 158 for sample collection. The securing mechanism 3010 comprises a collar 3011 and a sleeve 3021. The collar 3011 includes portions 3012 and 3014, which are configured in the same manner described above in relation to portions 512, 514 and portions to provide an interference fit with the open end at the base of the adaptor 150 and to prevent the collar 3011 from being advanced further into adaptor 150. The collar 3011 includes notched channels 3016 in the interior of collar 3011. Collar 3011 further includes detents or protrusions 3017 extending from an interior surface in each notched channel 3016. The sleeve 3021 has raised guides 3023 that travel through the notches 3016 in the adaptor collar 3011. The raised guides 3023 each include stepped or raised portions 3022. Sleeve 3021 includes raised tabs 3024 which extend toward the exterior of sleeve 3021. The sleeve 3021 is fitted onto the collection/discard tube 102. In one aspect, the sleeve 3021 is mounted to tube 102 via friction or gripping between the interior of sleeve 3021 and the exterior of tube 102 or via adhesive applied between the interior of sleeve 3021 and the exterior of tube 102. During shipment or storage, the raised portions 3022 sit behind detents 3017 such that the collection/discard tube 102 is kept in a stationary position and in spaced relation from the needle 158 and additional pushing force is required to advance tube 102 into the interior of adaptor 150. In one aspect, the raised portions 3022 have an interference fit with the interior surface of notched channels 3016 to also aid in retaining the tube 102 in the stationary position unless a pulling or pushing force is applied to tube 102. Blood is drawn into the collection/discard tube 102 when the needle in the adaptor 150 pierces the seal in the cap of the collection/discard tube 102 when it is advanced into contact with the needle 158 when the raised portions 3022 are forced to bypass the detents 3017 by the force on the tube 102. The channels 3016 and guides 3023 cooperate to provide smooth linear motion to tube 102. When the cap of tube 102 is advanced into adaptor 150 to engage needle 158, the tabs 3024 are configured to bend inward in a direction toward the interior of sleeve 3021 upon being compressed from the contact with the rim and interior surfaces of portions 3014 and 3012. As tub 102 is pushed, the tabs 3024 advance beyond portion 3012 into the interior of adaptor 150, where the tabs 3024 bend or deflect outwards to their original position due to the natural bias of tabs 3024. After sample collection, the collection/discard tube 102 is pulled to remove the tube 102 from the adaptor 150. When the tube 102 is pulled, the end of tabs 3024 abut against the rim defining the open end of portion 3012 of collar 3011 thus transferring the pulling force applied to tube 102 onto collar 3011 and forcing the collar 3011 to disengage from the adaptor 150 and be removed along with sleeve 3021 and tube 102 when the collection/discard tube 102 is removed from the adaptor 150.

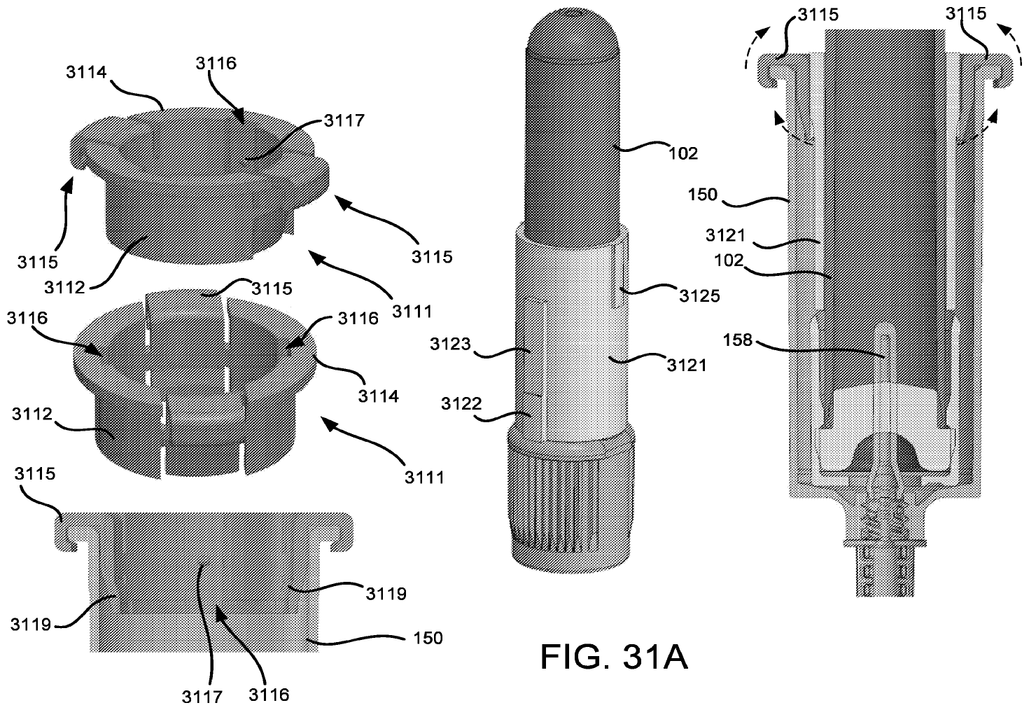

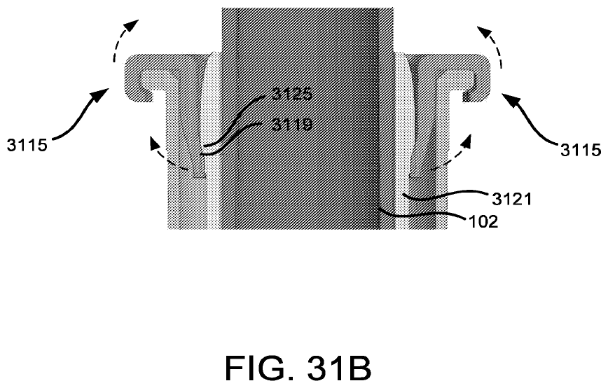

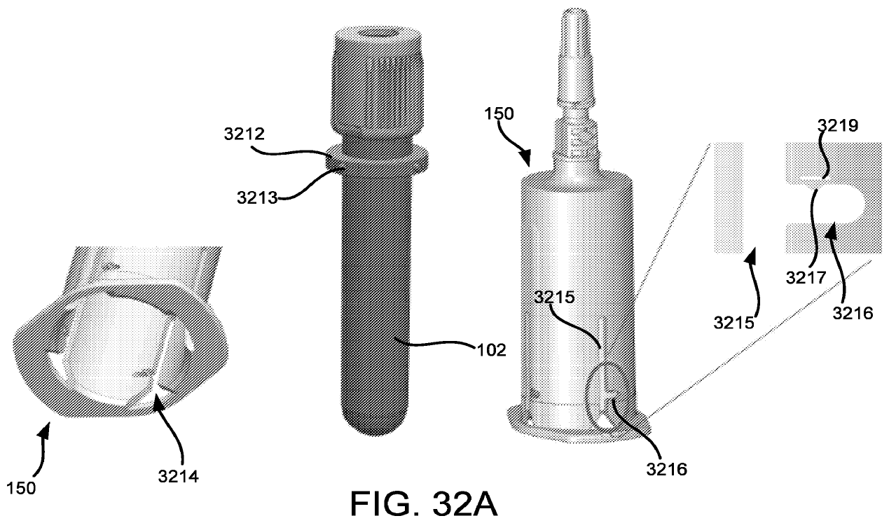

FIG. 31A-FIG. 31B illustrates a securing mechanism 3110 and is a modified aspect of what is illustrated in FIG. 30A-FIG. 30C. It is to be appreciated that components of FIG. 31A that are similarly numbered to components of FIGS. 30A-30C (i.e., 30XX vs. 31XX) are configured with the same features unless otherwise specified. The collar 3111 has clips 3115 that fasten to the flange at the base of the adaptor 150, as described above in previous embodiments. The interior surface of collar 3111 includes inclined surface or raised portions 3019, which converge toward the center of the collar 3111. Clips 3115 and raised portions 3019 are configured as deflectable tabs, where deflection of a first tab 3119 toward the exterior of collar 3111 in an arc causes deflection of a first clip 3115 toward the interior of collar 3111 in an arced motion. The same relationship holds true for a second tab 3119 and a second clip 3115, etc. The arced motion is shown by the arrows with dotted lines in FIGS. 31A-31B. The sleeve 3021 has raised guides 3121 that travel through the notches 3116 in the adaptor collar 3111 and detents 3117 that cooperate with raised portions 3122, as described above. The sleeve 3011 further includes raised portions 3125, which correspond to respective pairs of clips 3115 and tabs 3119, which as described below contact tabs 3119 to cause deflection of clips 3115. The sleeve 3121 is fitted onto the collection/discard tube 102. The sleeve has raised portions 3112 at the base of the sleeve 3112 that interact with detents 3117 ensure that the collection/discard tube 102 is held in spaced relation from the needle 158 during shipment and storage. Blood is drawn into the collection/discard tube 102 when the needle 158 in the adaptor 150 pierces the seal in the cap of the collection/discard tube 102 when it is advanced into contact with the needle 102 by bypassing the detents 3117. When the tube 102 is advanced into adaptor 150 and into engagement with needle 158, raised portions 3125 deflect raised portions 3119, which cause deflection of clips 3115, as indicated by the dotted lines and arrows in FIGS. 31A-31B. When clips 3115 are deflected in this manner, the clips 3115 disengage the flange of the base of the adaptor 150. When the raised portions 3125 deflect the raised portions 3119, portions 3125 and 3119 may form an interference fit such that after sample collection, when the collection/discard tube 102 is pulled and removed from the adaptor 150, the collar 3111 with disengaged clips 3115 and sleeve 2131 along with the tube 102 are also removed from adaptor 150. The disengaged clips 3115 permit the withdrawal.

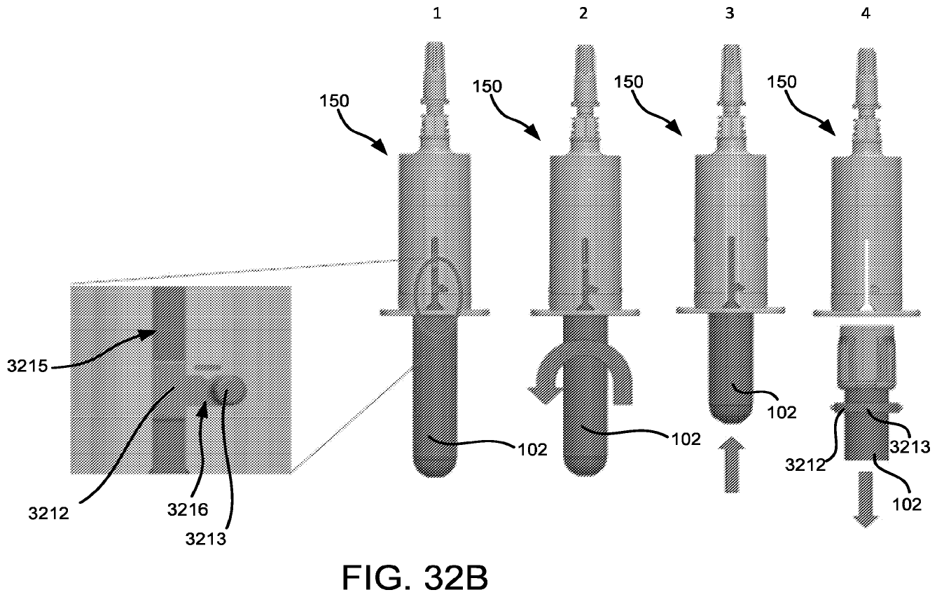

FIG. 32A-FIG. 32B is a modified aspect of an assembly for holding the collection/discard tube 102 in spaced arrangement from the needle 158 in the adaptor 150 during shipment but allowing the collection/discard tube 102 to be advanced into engagement with the needle 158 for sample collection. In this aspect, the securing mechanism 3210 comprises a ring or collar 3212 and a slot 3214 on the adaptor 150. The collar 3212 is disposed on the collection/discard tube 102 and has posts 3212 that each engage a slot 3214 in the adaptor housing 150. It is to be appreciated that although multiple slots and posts are shown, in some aspects, a single slot and post may be used. The side slot 3214 includes portions 3215 and 3216. Portion 3215 extends along a longitudinal direction parallel to the insertion and extraction direction of the tube 102 into adaptor 150. Portion 3216 branches off from portion 3215 in a direction that traverses the longitudinal direction. Portion 3215 of the slot is configured to receive a post 3213 and allows the collar and tube 102 to advance longitudinally within portion 3215 of the slot 3214. Portion 3216 receives post 3213 to allow the collar 3212 (and tube 102) to be rotated into a locked position (i.e., preventing longitudinal motion of collar 3212 and tube 102) that locks the collection/discard tube 102 in spaced relation from the needle 158 during shipment and storage. In one aspect, portion 3216 of slot 3214 includes a detent or protrusion 3217 that extends into portion 3216 to lock the post 3213 in portion 3215. A slot 3219 behind detent 3217 allows detent 3217 to move longitudinally when post 3213 is formed to traverse (e.g., by a torque force applied by the user to tube 102) through portion 3216 of slot 3214. To advance the collection/discard tube 102 into engagement with the adaptor needle 158, the collection/discard tube 102 is rotated/twisted such that post 3213 travels through portion 3215 and into portion 3215 of slot 3214 and then tube 102 is pushed longitudinally to allow post 3213 to travel longitudinally through portion 3215 until tube 102 is advanced into engagement with the needle 158 in the adaptor 150 for blood collection because the collar pin/post 3213 can again advance along the slot portion 3215 in the adaptor housing 150. After sample collection, the collection/discard tube 102 is removed from the adaptor 150 by pulling tube 102, thereby pulling post 3213 through slot portion 3215 along with the collar 3212 until the tube 102 and collar 3212 are removed from adaptor 150. It is to be appreciated that collar 3212 may be fixedly coupled to tube 102 or may have an interference or friction fit with tube 102 such that collar 3212 and tube 102 remain stationary with respect to each other.

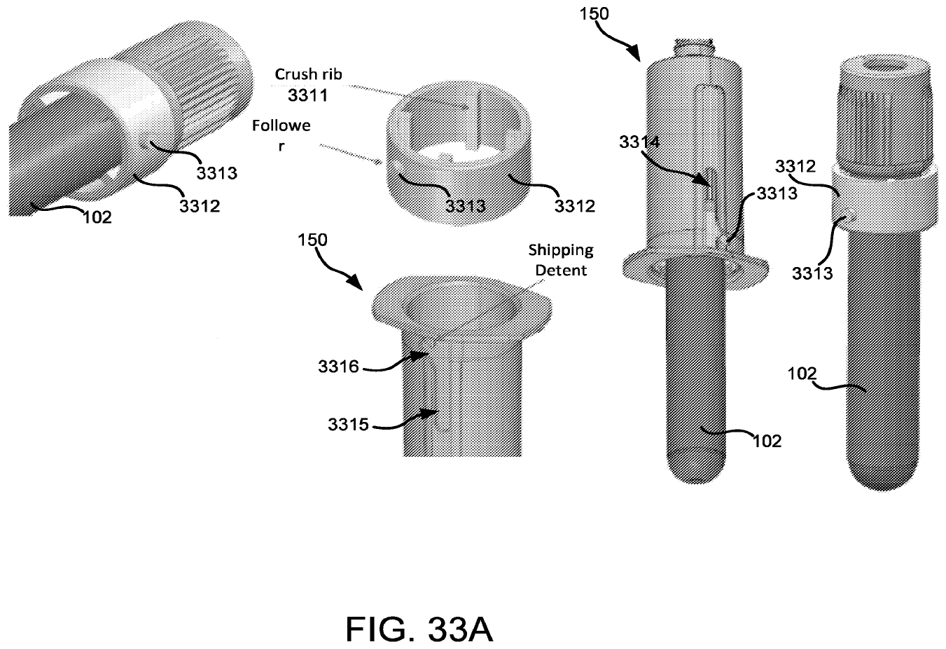

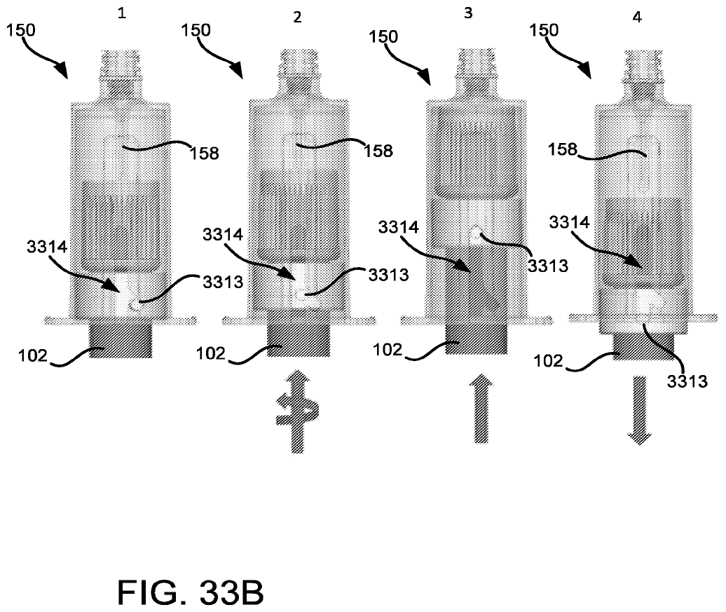

FIG. 33A-FIG. 33B is a modification of the aspect of FIG. 32A-FIG. 32B. The securing mechanism in FIG. 33A-33B comprises collar 3312 and slot 3314 in adaptor 150. Collar 3312 includes posts 3313 which are slidable in slot 3314, which includes slot portions 3316, 3315. Slot portion 3315 extends longitudinally and slot portion 3316 branches off in a direction traverse to the longitudinal direction. The collar 3312 that is disposed on the collection/discard tube 102 has posts 3313 that engage an angled slot 3316 in the adaptor housing 150. The side slot 3316 receives the follower pin/post 3313 on the collar 3312 and holds the collection/discard tube 102 in spaced relation to the needle 158 in the adaptor 150. The slot portion 3316 is angled such that the post 3313 when sitting at the closed end of portion 3316 prevents tube 102 from being pulled out of adaptor 150. At the same time, slot portion 3316 is angled relative to portion 3315 such that when the operator advances the collection/discard tube 102 longitudinally, the follower pin/post 3313 is permitted to move through and past the detent into slot portion 3315, which causes the collection/discard tube 102 to twist such that the follower pin/post 3313 is in the main vertical slot 3315. Pin 3313 in slot 3315 allows tube 102 to be advanced longitudinally into adaptor 150 and into engagement with needle 158. After sample collection, the collection/discard tube 102 is removed from the adaptor 150 by pulled tube 102. Tube 102 is removed along with the collar 3312. The follower pin/post 3313 moves down the vertical main slot 3315 for removal of the collection/discard tube 102 from the collection/discard device 150.

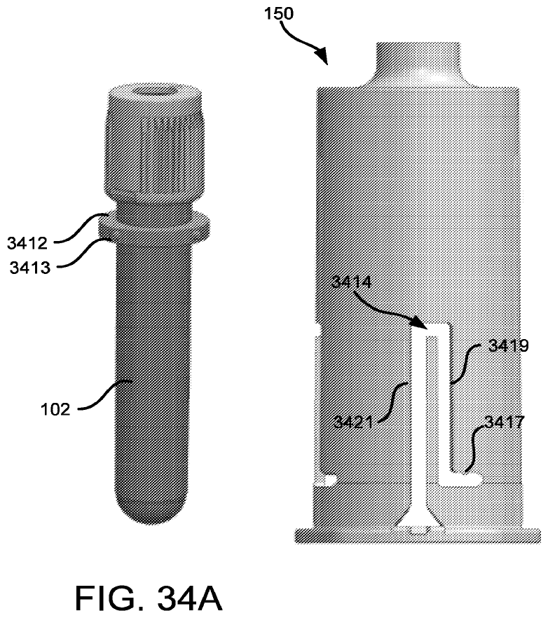

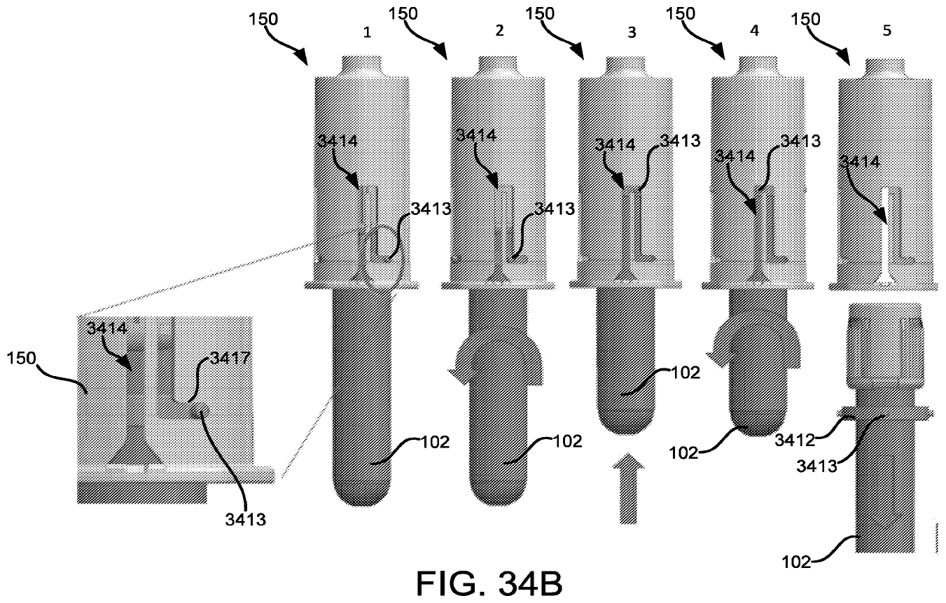

FIG. 34A-FIG. 34B is a modification of aspects of FIG. 32A-FIG. 32B. The securing mechanism 3410 in FIGS. 34A-34B include a collar 3412 with posts 3413 and a slot 3414 in the adaptor housing 150. The slot 3414 is configured in a “U-turn” shaped path and forces the user to collect sample in the collection/discard tube 102 before the tube 102 is removed from the adaptor 150. The slot may include a detent or protrusion 3417. The configurations described herein that require the collection/discard tube 102 to engage the needle 158 in the adaptor 150 before the collection/discard tube 102 is removed therefrom are referred to herein as forced compliance configurations. The collar 3412 that is disposed on the collection/discard tube 102 has follower posts 3413 that engage a slot 3414 in the adaptor housing 150. The side slot allows the collar 3414 to be rotated into a locked position that locks the collection/discard tube 102 in spaced relation from the needle during shipment and storage. A detent or protrusion 3417 may keep the post 3413 in the locked position. To advance the collection/discard tube 102 into engagement with the adaptor needle 158, the collection/discard tube 102 is rotated such the post 3413 is also rotated past detent 3417 and the collar/collection/discard tube assembly can be advanced longitudinally (i.e., with post 3413 travelling along first longitudinal portion 3419 of slot 3414 to the end of portion 3419) into engagement with the needle 158 in the adaptor 150 for blood collection because the collar follower pin 3413 can again advance along the slot 3419 that is adjacent to the side slot in the adaptor housing 150. After sample collection, the collection/discard tube 102 is again rotated and the collar pin 3413 travels down a discard slot longitudinal portion 3421, thereby removing the collection/discard tube 102 from the adaptor 150 along with the collar 3412. To follow this path, the collection/discard tube 102 must be brought into engagement with the needle 158 in the adaptor 150.

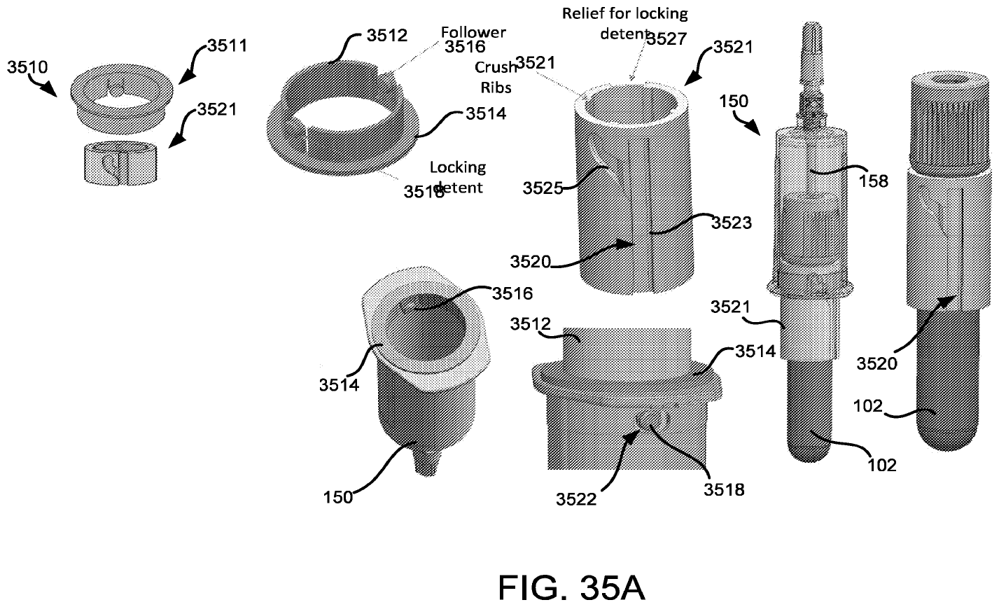

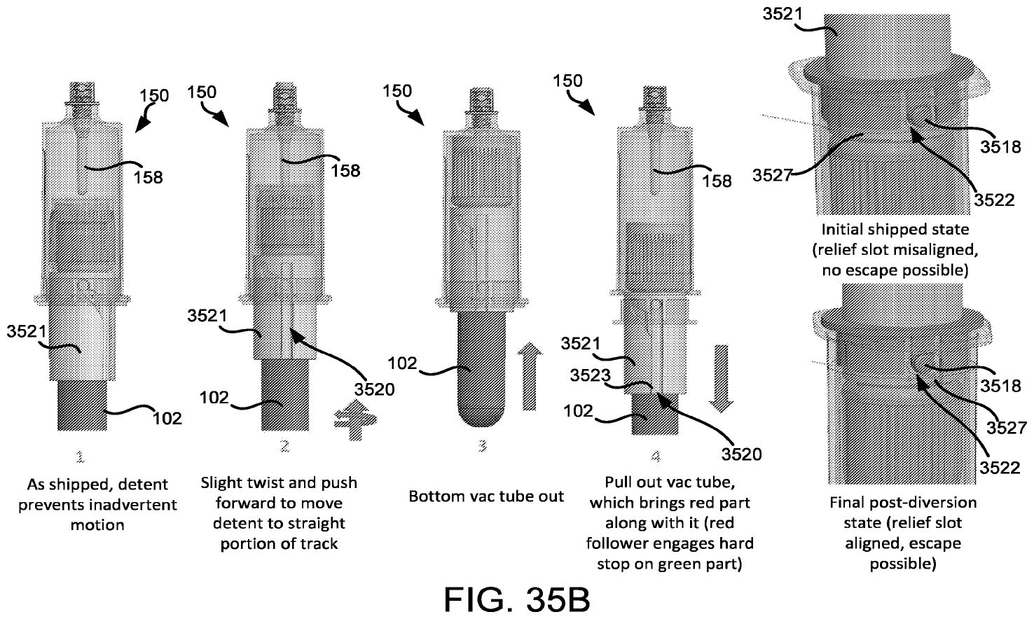

FIG. 35A-FIG. 35B is a modification of the aspects of FIG. 33A-FIG. 33B. The securing mechanism 3510 in FIGS. 35A-35B comprises a collar 3511 and a sleeve 3521. Collar 3511 includes portions 3512 and 3514. Portion 3512 is configured to be received by the open base end of the adaptor 150. Portion 3514 is configured to abut against the flanged base of adaptor 150 to stop the securing mechanism 3510 from being advanced further into the interior of adaptor 150. Portion 3512 includes pins or posts 3516, 3518. Post 3516 extends toward the interior of portion 3511 and post 3518 extends toward the exterior of portion 3511. Posts 3516, 3518 are configured to be able to bend radially inward toward the center of collar 3511. As will be described below, post 3516 controls the motion of the collection/discard tube 102 relative to the adaptor 150.

Sleeve 3521 includes a ribbed interior (e.g., including ribs 3521 that extend from an internal surface of sleeve 3521 for gripping tube 102). The ribbed sleeve 3521 fits snugly on the collection/discard tube 102. When portion 3512 is inserted into adapter 150 and portion 3514 abuts against adaptor 150, the locking detent pin 3518 locks into and extends into a slot 3522 in the adaptor 150 to hold the collar 3511 in a stationary position relative to adaptor 150. Sleeve 3521 includes a relief or slot 3527 in the exterior surface of sleeve 3521. Sleeve 3521 further includes a slot 3520 with a longitudinal portion 3523 and an angled portion 3525 that branches from portion 3523. Pin 3516 is slidably disposed in slot 3520. When the collection/discard tube 102 is rotated such that the post 3516 is engaged in end of the angled slot 3525, this locks the collection/discard tube 102 in spaced relation from the needle 158 during shipment and storage and prevents (i.e., by pin 3516 abutting the closed end of portion 3525) the tube 102 or collar 3511 from advancing further into adaptor 150. As shown in FIG. 35B, in this position, the relief slot 3527 on the exterior of sleeve 3521 is not aligned with post 3518 and thus it is not possible for the securing mechanism to escape adaptor 150. To advance the collection/discard tube 102 into engagement with the adaptor needle 158, the collection/discard tube 102 is pushed and rotated and guided by the travel of pin 3516 in portion 3525 and into portion 3523 of slot 3520 such that the collar/collection/discard tube assembly may be advanced into engagement with the needle 158 in the adaptor 150 for blood collection because the collar pin 3516 can again advance along portion 3523 in the slot 3520 in the sleeve 3521. After sample collection, the collection/discard tube 102 is pulled and guided by pin 3516 in sleeve 3520 and the sleeve 3521 travels downward because the collar pin 3516 is aligned with the slot 3520 in sleeve 3521. When the collection discard tube 102 is removed from the adaptor 150, the pin 3516 engages the closed end (hard stop) of portion 3523 of slot 3520 and the relief slot 3527 is aligned with pin 3518 allowing pin 3518 to bend radially inward and exist slot 3522 in adaptor 150. Thus, the collar 3511 is not locked with the adaptor 150 and is pulled out of the adaptor 150 with tube 102 when pin 3516 engages the hard stop at the closed end of portion 3523 of slot 3520.

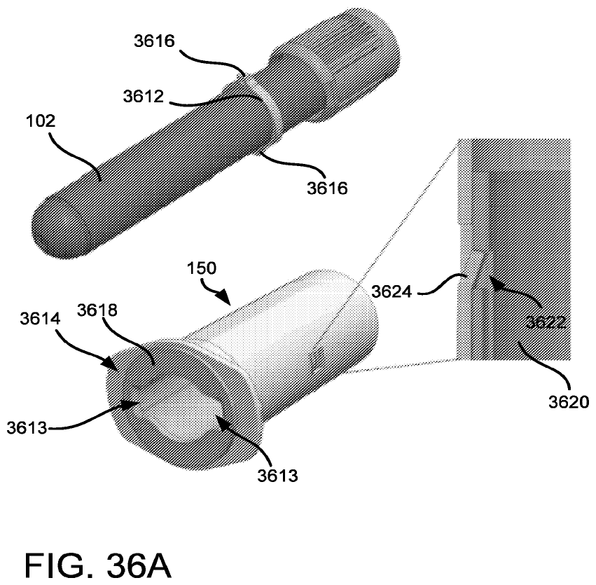

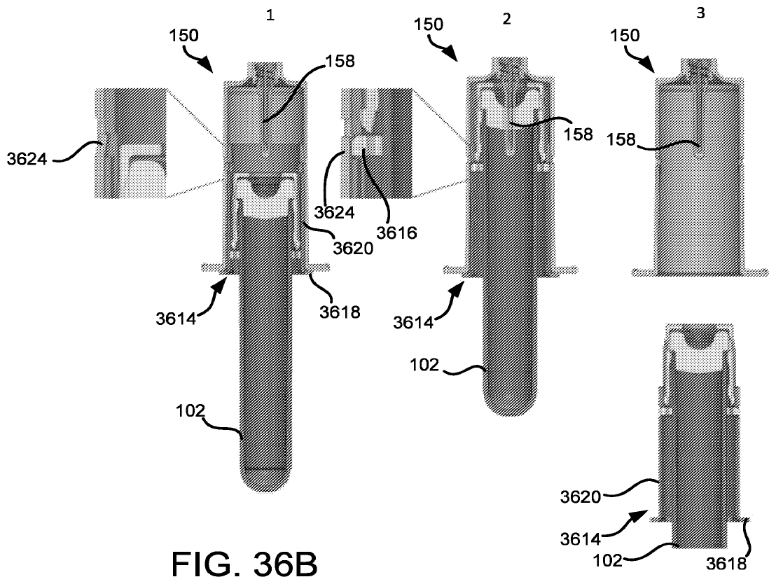

FIG. 36A-FIG. 36B is another aspect of an assembly with a securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In this aspect, the securing mechanism comprises a collar 3612 and an insert 3614. The collar 3612 is disposed around the tube 102 and includes flexible fingers or posts 3616 that extend toward the exterior of the tube 102. The insert 3614 includes a barrel or tubular portion 3620 that is received in the interior of adaptor 150. Insert 3614 further includes a flanged end portion 3618 that has a larger diameter than the inner diameter of adaptor 150 and thus prevents the insert 3618 from being advanced further into adaptor 150. Insert 3614 further includes slots 3622 in portion 3620. In this aspect, adaptor 150 includes flexible or deflectable tabs 3624 that are biased to tilt toward the interior of adaptor 150 and are received by corresponding slots 3622 of the insert 3614 to lock the insert 3614 in a stationary position with respect to the adaptor 150. When the assembly is shipped or stored, the tube 102 and collar 3612 is held in a spaced relationship with needle 158. In one aspect, the finger 3616 press into the interior surface of insert 3614 such that the tube 102 is held in the spaced relationship unless a force is applied. In one aspect, insert 3614 includes longitudinal grooves 3613 that the posts 3616 travel or slide in longitudinally when the tube 102 is pushed into adaptor 150. The collection/discard tube 102 may advance into engagement with the needle 158 in the adaptor 150. When the tube 102 is pressed into adaptor 150, the fingers 3616 enter the slots 3622 and force the tabs 3624 out of the slots 3622 to release the insert 3614 from the locked arrangement with the adaptor 150. After sample collection, the collection/discard tube 102 is pulled and removed from the adaptor 150 and the fingers 3616 in slots 3622 pull the insert 3614 along with the tube 102 and the collar 3612 out of adaptor 150.

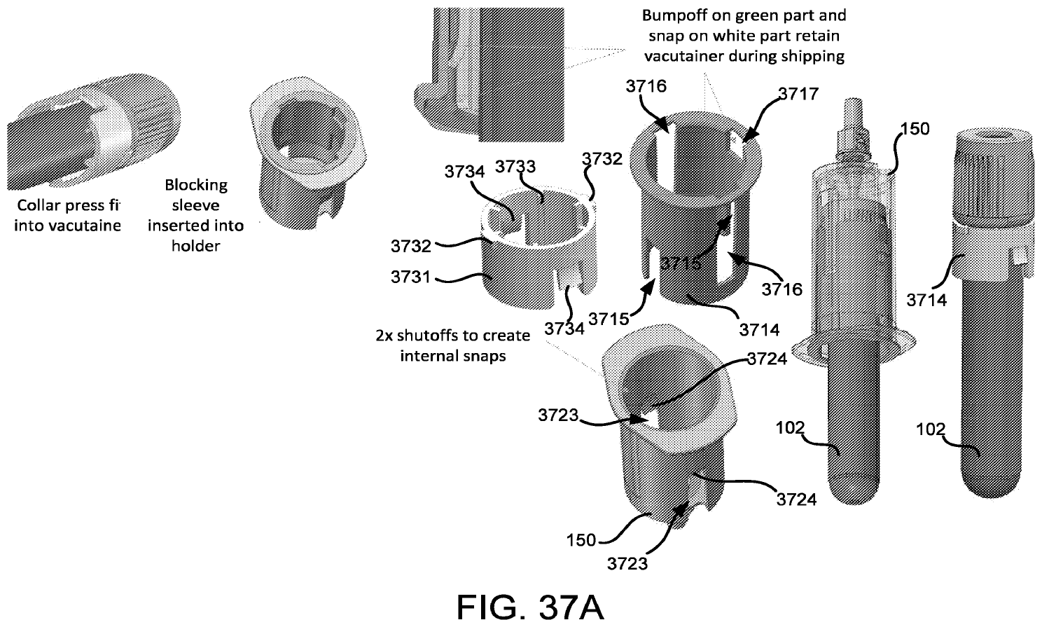

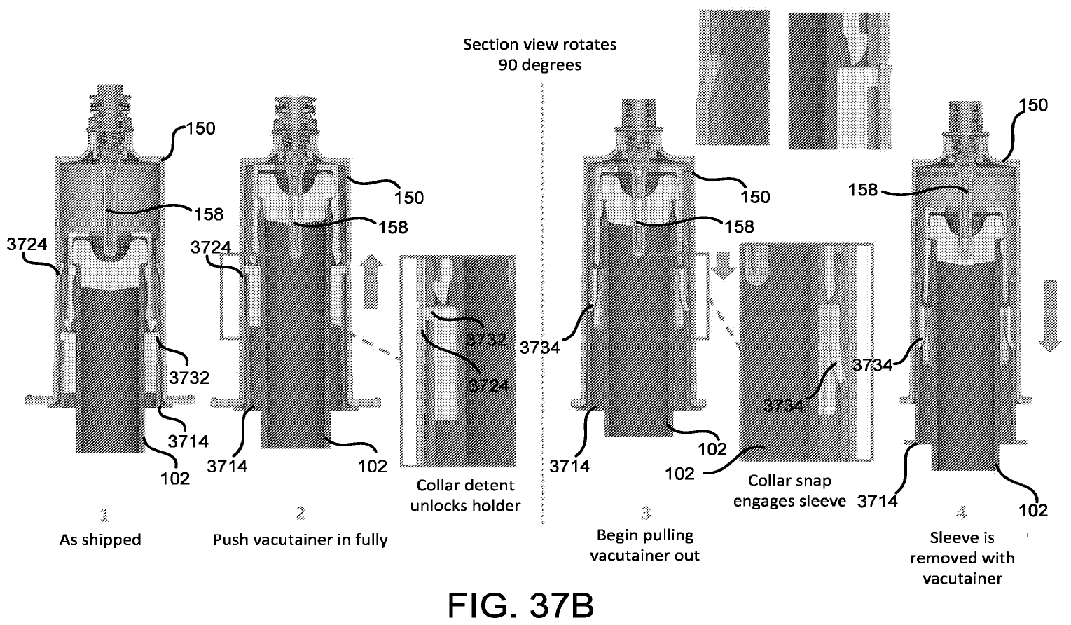

FIG. 37A-FIG. 37B is another aspect of an assembly illustrated in FIG. 36A-36B including a securing mechanism that that keeps the collection/discard tube 102 in spaced relation from the adaptor needle 158 during shipping and storage and will allow the collection/discard tube 102 to advance into engagement with the needle 158 in the adaptor 150 for sample collection. In the illustrated aspect, the securing mechanism has a sleeve 3714 with a bump off 3717, slots 3715, and slots 3716. The securing mechanism further comprises a collar 3731 including raised portions 3732 and flexible projections or fins 3734 that are biased to project at an angle toward the exterior of collar 3731. The collar 3731 includes ribs 3733 to grip the exterior of tube 102 as shown. The sleeve 3714 is received into adaptor 150 as shown and includes a flanged end to prevent the sleeve 3731 from advancing further into adaptor 150 than what is shown. The adaptor includes flexible projections or fins 3724 that are biased to project toward the interior of adaptor 150. When the sleeve 3714 is inserted into adaptor 150, the projections 3724 extend into and catch and end of respective slots 3716 to retain sleeve 3714 in a locked position in adaptor 150. The collar 3731 grips the tube 102 as shown and tube 102 is inserted into sleeve 3714 such that projections 3734 project into and catch slots 3717 to retain tube 102 and collar 3731 in a locked position such that tube 102 is in a spaced relationship with needle 158 of adaptor 150. When tube 102 is advanced into adaptor 150, the raised portions 3716 are guided along slots 3716 and the projections 3734 deflect inward to release collar 3731 from the locked position. In this way, the collection/discard tube 102 may advance into engagement with the needle 158 in the adaptor 150. When the tube 102 engages the needle 158, the raised portions 3732 press projections 3724 outward and out of slots 3716, thus unlocking sleeve 3714 from adaptor 150. Furthermore, projections 3734 extend into slots 3715 and catch the closed end of slots 3715, thus locking collar 3731 to the sleeve 3714. After sample collection, the collection/discard tube 102 is removed from the adaptor 150 along with the sleeve 3714 and the collar 3731, which are now engaged.

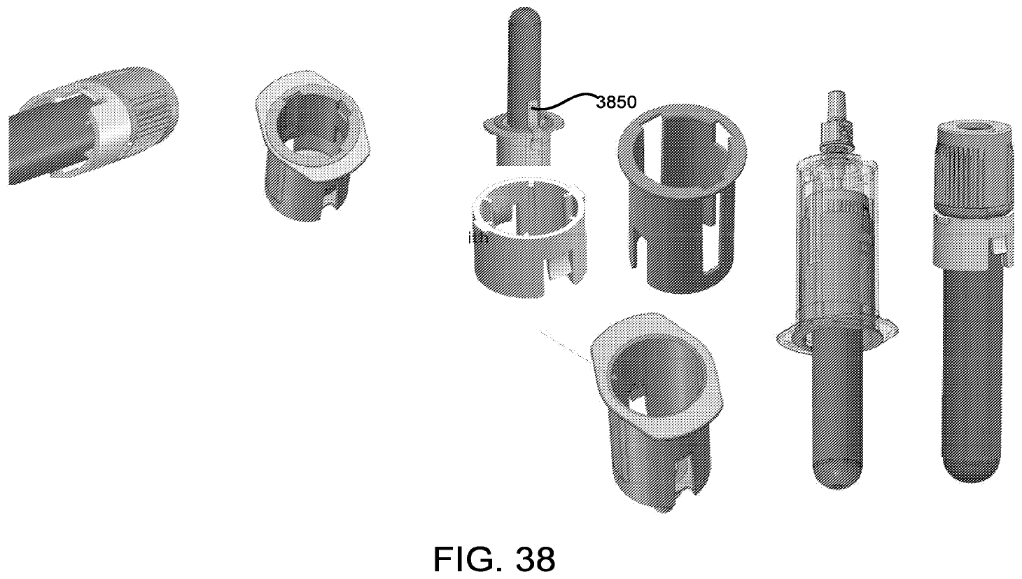

FIG. 38 is the aspect of FIG. 37A-FIG. 37B but with tape or sticker 3850 instead of relying on the bump out to lock the collection/discard tube into distanced position from the needle in the adaptor during shipping and storage.

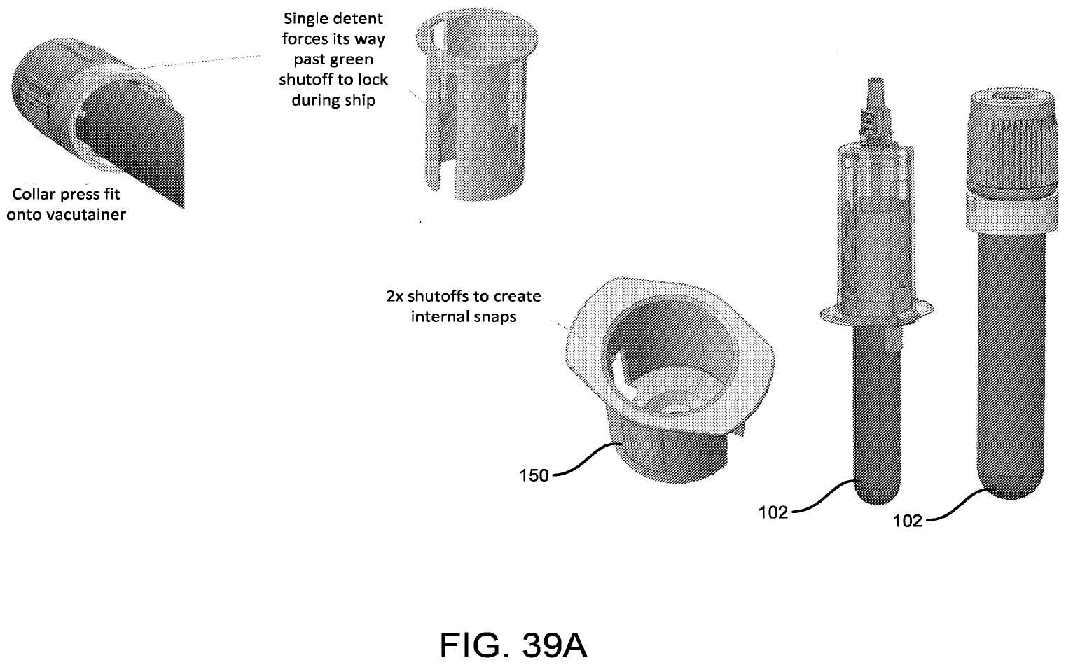

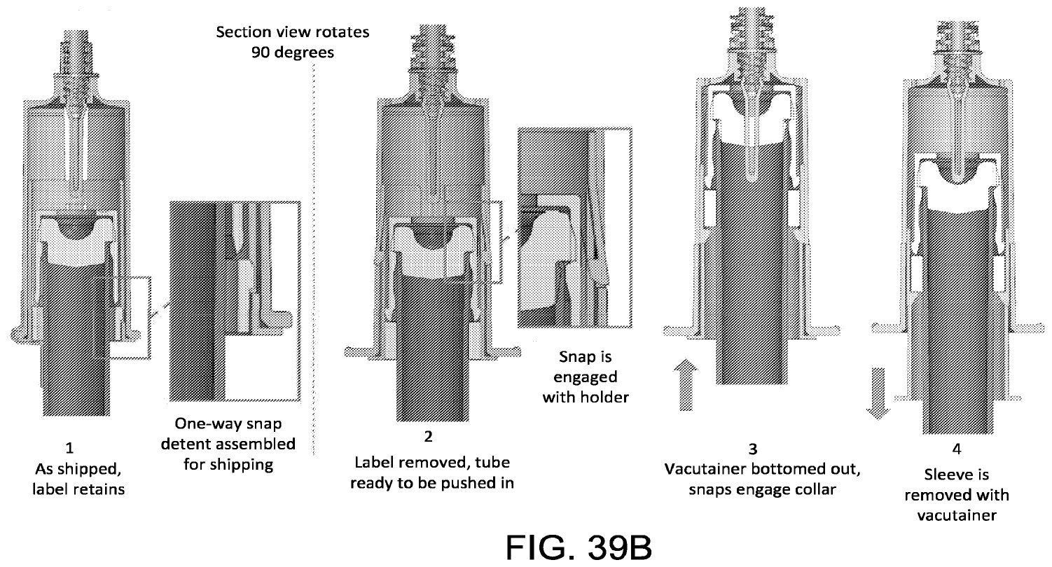

FIG. 39A-FIG. 39B is another aspect of an assembly illustrated in FIG. 36A-36B including a securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In the illustrated aspect, the adaptor has a sleeve with a resilient stop that will allow a projection on a collar that is press fit onto the collection/discard tube to pass by it when force is applied to advance the collection/discard tube in the adaptor. As such, tape is applied to ensure that the collection/discard tube is retained in the desired spaced relation with the adaptor needle. When the tape is removed, the collection/discard tube may advance into engagement with the needle in the adaptor. The collar also has detent projections that travel along guides in the adaptor sleeve. At this point, the projections on the collar and the resilient stop engages the projections on the collar. After sample collection, the collection/discard tube is removed from the adaptor along with the adaptor sleeve, which is engaged with the collar.

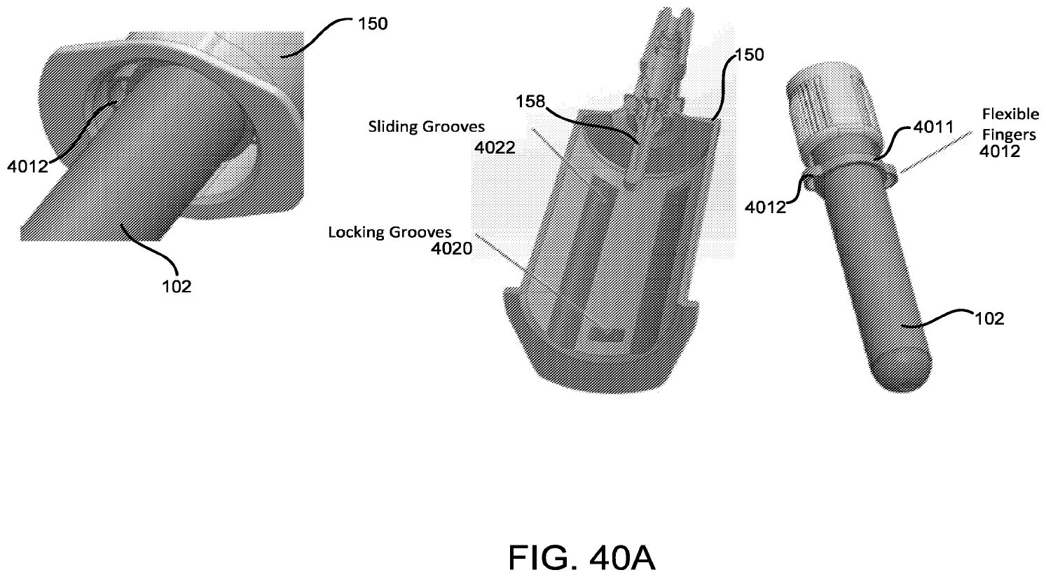

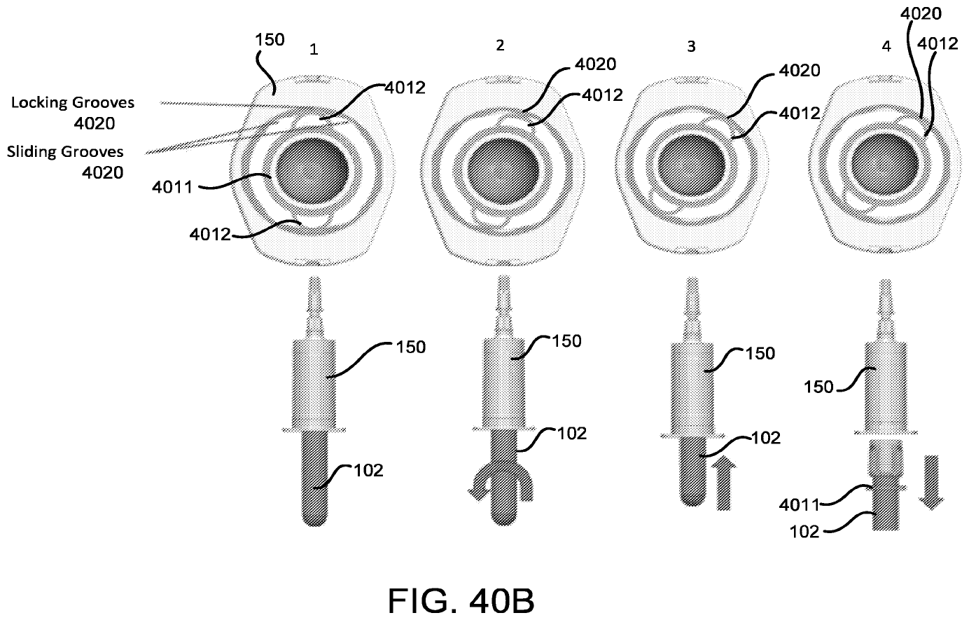

FIG. 40A-FIG. 40B is another aspect of an assembly with a securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In the illustrated aspect, the adaptor has longitudinal sliding grooves 4022 and locking grooves or slots 4020 disposed in the interior surface of the adaptor 150. Moreover, the securing mechanism comprises a collar 4011 fixed or coupled to the exterior of the tube 102. The collar 4011 includes flexible fingers or projections 4012 extending from a ring portion of the collar 4012. The flexible fingers 4012 on the collar 4012 extend into the respective locking grooves 4020, where tube 102 is in a spaced relationship with the needle 158 (i.e., not engaged therewith) for storage and/or shipment and the grooves 4020 and flexible fingers 4012 prevent insertion/removal of the tube 102 into or out of the adaptor 150. The flexible fingers 4012 are configured to be depressed when the collar 4012 and tube 102 is rotated. Thus, the fingers 4012 allow rotation of the tube 102 such that rotation of tube 102 causes the fingers 4012 to exit the locking grooves 4020 and to enter the sliding grooves 4022. When collection/discard tube 102 is rotated to place the fingers 4012 in the sliding grooves 4020 of the adaptor 150, the collection/discard tube 102 may be advance into engagement with the needle 158 in the adaptor 150 with the fingers 4012 sliding longitudinally in the sliding grooves 4022 to permit the advancement. After sample collection, the collection/discard tube 102 is removed from the adaptor 150 by sliding it out with the flexible fingers 4012 sliding along the sliding grooves 4022.

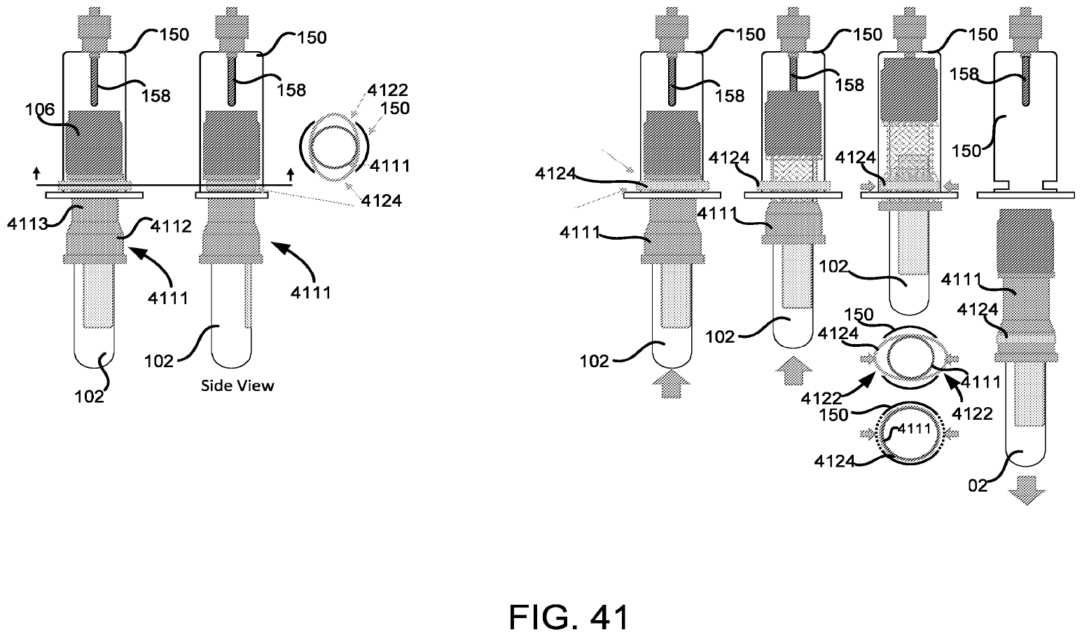

FIG. 41 is another aspect of an assembly with a securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In this aspect, the securing mechanism is configured such that the collection/discard tube 102 must be placed into engagement with the needle 158 in the adaptor 150 before it is removed from the adaptor 150. As such, this aspect is also a forced compliance configuration. In the illustrated aspect, the collection/discard tube 102 has a sleeve 4111 attached thereto with a portion 4112 of the sleeve 4111 having an enlarged diameter relative to a portion 4113. The sleeve 4111 has a flange at the proximal end that prevents the sleeve from being advanced entirely into the adaptor 150. The adaptor 150 has windows 4122 cut into the sides of the housing that hold a molded, flexible component 4124 (e.g., an oval locking ring that is free floating) in an oval configuration. The component 4124 is in the oval shape when in a resting state. The oval component 4124 holds the collection/discard tube 102 in spaced relation to the adaptor needle 158. In this regard, in one aspect, the inner circumference of the ring 4124 along the minor axis (i.e., the vertices of the minor axis) grip the outer surface of the sleeve 4111 at portion 4113 to hold (via friction) the tube 102 in the spaced relationship. The larger diameter portions of the oval component 4124 protrude into/from windows 4122 in the base of the adaptor 150 and this oval component 4124 locks the collection/discard tube 102 in place such that it is maintained in spaced relation from the needle 158 during shipping and storage. When the collection/discard tube 102 is forced past the oval component 4124, the tube 102 can be advanced into contact with the adaptor needle 158 for sample collection. In that position, the wider portion 4112 of the sleeve 4111 changes the oval component 4124 configuration to circular (i.e., the outer surface of portion 4112 contacts and pushes out against the inner circumference at the minor axis or shorter diameter of the oval component 4124 to change the shape to circular), which pulls those portions of the oval component 4124 that were extending out of the windows 4122 at the base of the adaptor 150 inside the adaptor 150. In this position, the component 4124 is retained snugly by friction on the outer diameter of portion 4112. This change in shape allows the collection/discard container 102 to be backed out from the adaptor 150, carrying the component 4112 with the tube 102 and the sleeve 4111.

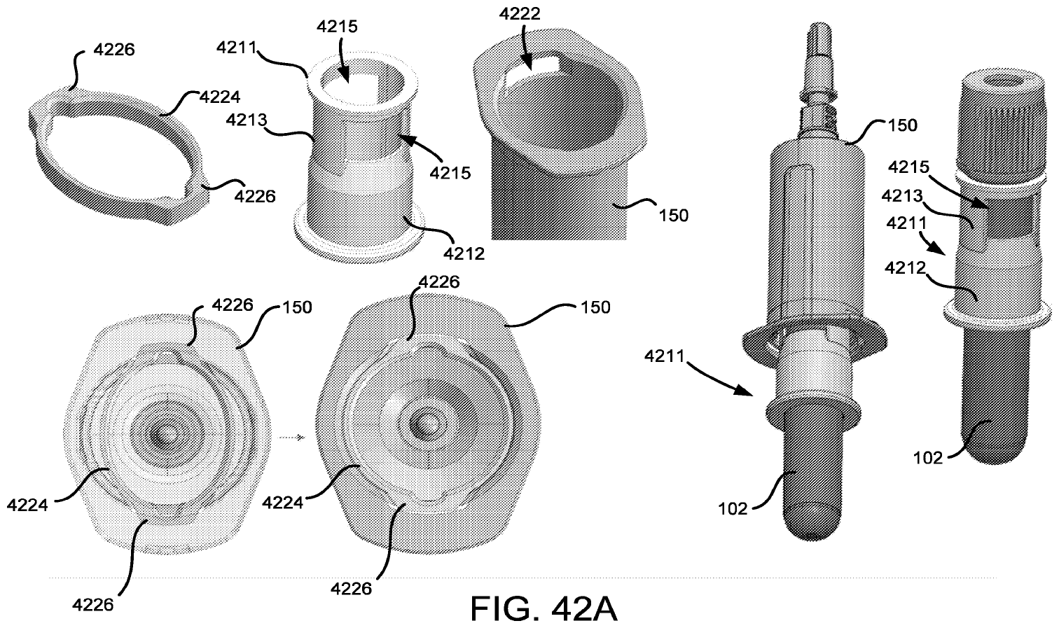

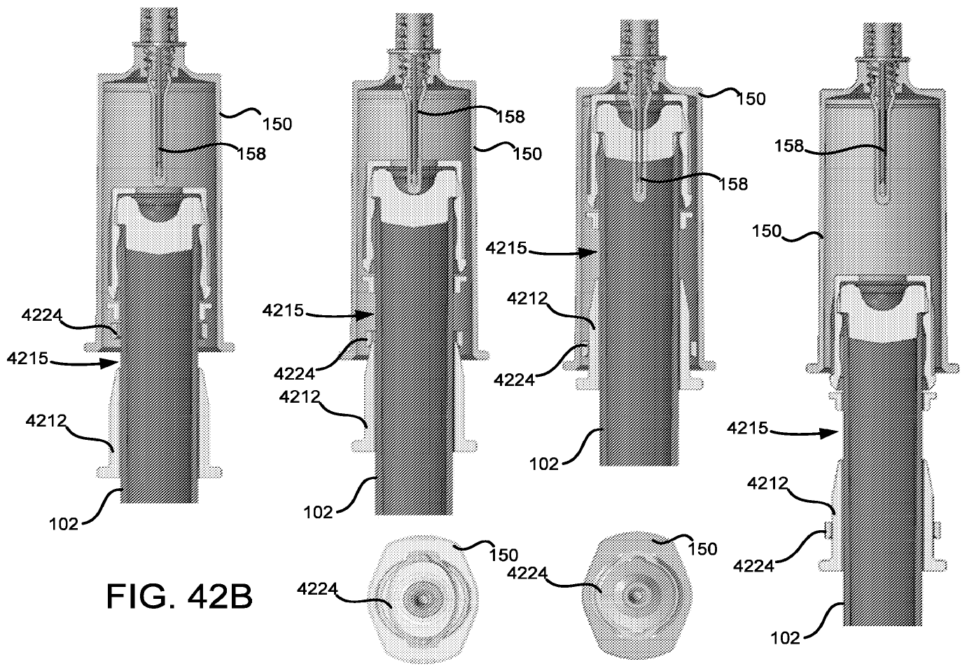

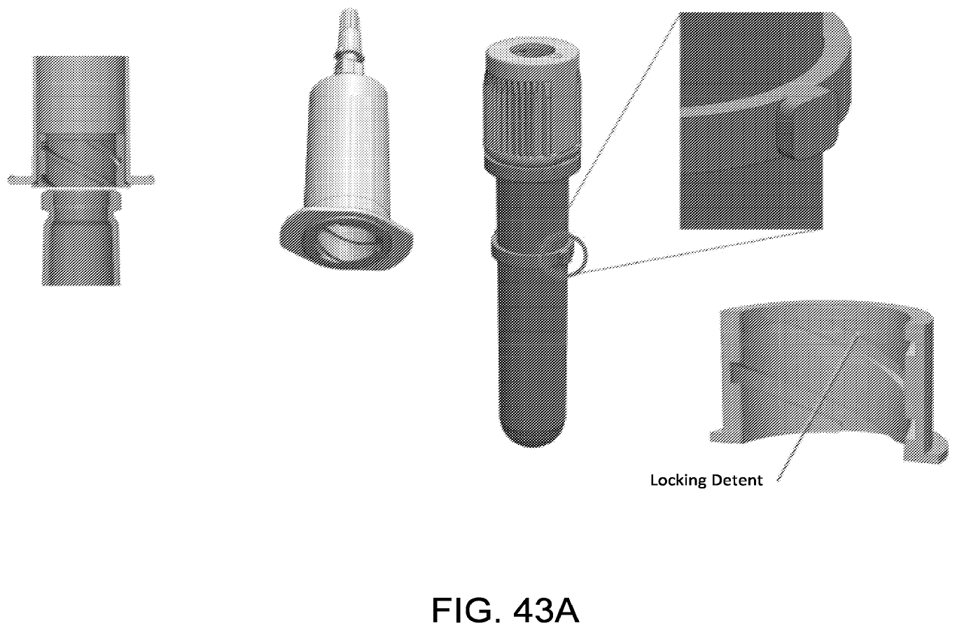

FIG. 42A-FIG. 42B is another aspect of the assembly of FIG. 41 including a securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In this aspect, the collection/discard tube 102 must be placed into engagement with the needle 158 in the adaptor 150 before it is removed from the adaptor 150. As such, this aspect is also a forced compliance configuration. In the illustrated aspect, the collection/discard tube 102 has a sleeve 4211 attached thereto with a portion 4212 of the sleeve 4211 having an enlarged diameter relative to a portion 4213. The adaptor 150 has windows 4222 above the flange at the base thereof that hold a flexible oval ring 4224. The oval ring 4224 includes tabs or projections 4226 at the vertices of the major axis (largest diameter) of the oval ring 4224. The tabs or projections 4226 are shaped to be received into windows 4222. The larger diameter portions (i.e., tabs 4226) of the oval component 4224 protrude from windows 4222 in the base of the adaptor 150 and the inner circumference portions of the ring 4224 along the minor axis (i.e., the vertices of the minor axis) extend into windows 4215 of sleeve 4211. In this way. the oval component 4224 locks the collection/discard tube 102 in place such that it is maintained in spaced relation from the needle 158 during shipping and storage. The oval ring 4224 holds the collection/discard tube 102 (by engaging both windows 4222 and 4215) in spaced relation to the needle 158 in adaptor 150. When the collection/discard tube 102 is forced through the oval ring 4224 into the interior of adaptor 150, the tube 102 can be advanced into contact with the adaptor needle 158 for sample collection. In that position, in the same manner described above in relation to FIG. 41, the wider portion 4212 of the sleeve 4211 changes the oval ring 4224 configuration to circular, which pulls the tabs 4226 that were extending into the windows 4222 at the base of the adaptor 150 inside the adaptor 150 and the portions of the inner circumference of ring 4224 that were extending into windows 4215 out of windows 4215. This change in shape allows the collection/discard container 102 to be backed out from the adaptor 150, carrying the ring component 4224 and sleeve 4211 with it.

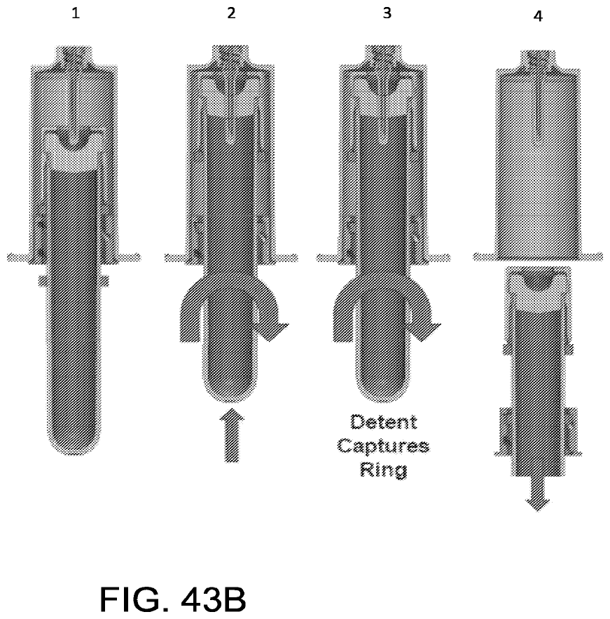

FIG. 43A-FIG. 43B is another aspect of an assembly with a securing mechanism that keeps the collection/discard tube in spaced relation from the adaptor needle during shipping and storage and will allow the collection/discard tube to advance into engagement with the needle in the adaptor for sample collection. In the illustrated aspect, the adaptor has a threaded inner collar with a flange that abuts the flange at the base of the adaptor. The collection/discard tube has a ring with posts. When the collection/discard tube is placed into the adaptor, the collection/discard tube is held away from the adaptor needle. When sample collection is desired, the collection/discard tube is rotated, which advances the ring, threading it such that the ring posts land in the detents in the threaded inner collar. When engaged, the sample is collected. After sample collection, the sample collection/discard tube is pulled from the adaptor with the ring and the threaded inner collar.

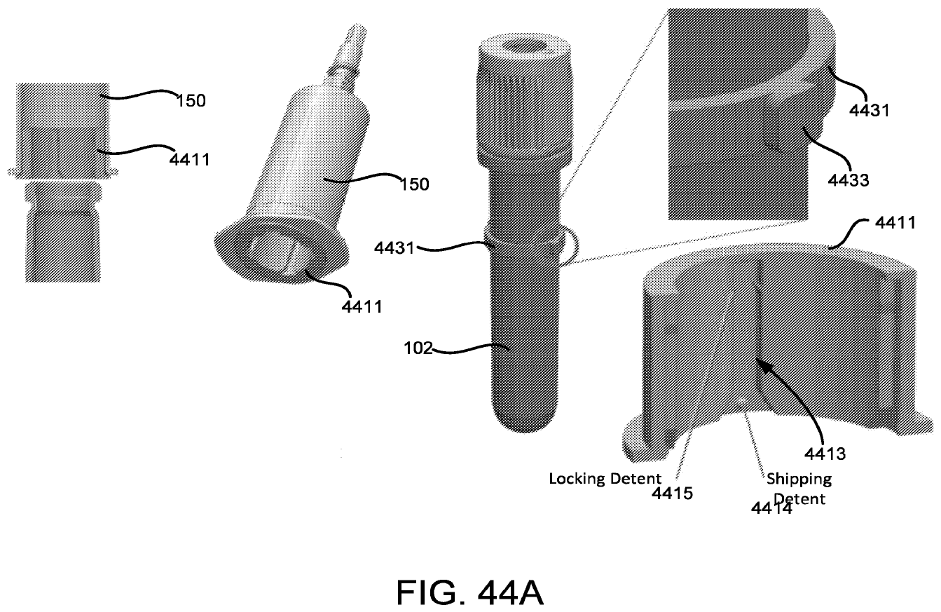

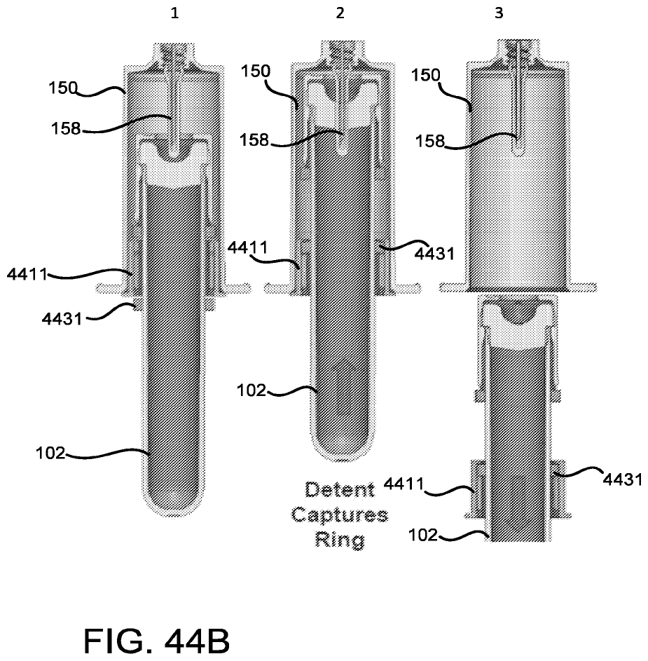

FIG. 44A-44B is an alternative aspect of FIG. 43A-FIG. 43B, and is another aspect of an assembly with a securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In the illustrated aspect, the securing mechanism comprises a slotted inner collar 4111 with a flanged end that abuts the flange at the base of the adaptor 150 and prevents the collar 4111 from being inserted further into the adaptor 150. The barrel portion of the collar 4111 is inserted into adaptor 150. The slot 4413 of the collar 4111 has two detents 4415, 4415. The collection/discard tube has a ring 4431 affixed thereon with posts 4433 that extend from a ring base. When the collection/discard tube 102 is placed into the adaptor 150, the collection/discard tube 102 is held away from the adaptor needle 158 by the engagement of each post 4433 with each first detent 4414. When sample collection is desired, the collection/discard tube 102 is advanced into adapter 150, such that the ring posts 4433 land in the locking detents 4415 at the top of slot 4413. When engaged, the sample is collected into the collection/discard tube 102. After sample collection, the sample collection/discard tube 102 is pulled from the adaptor 150 with the ring 4431 and the slotted inner collar 4411.

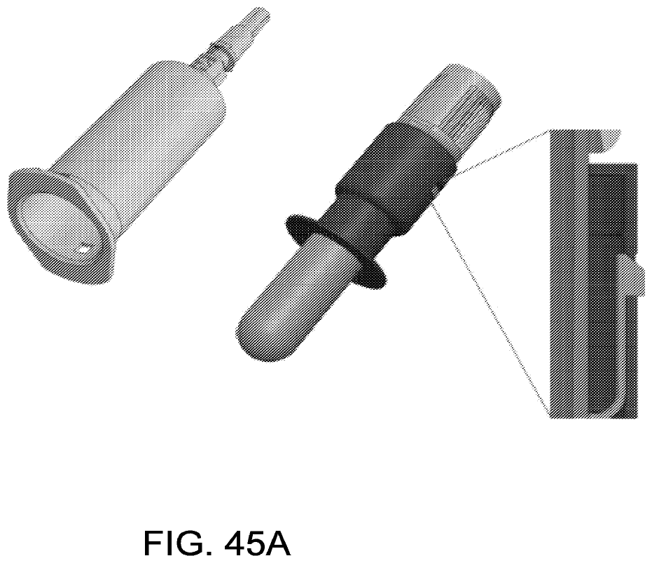

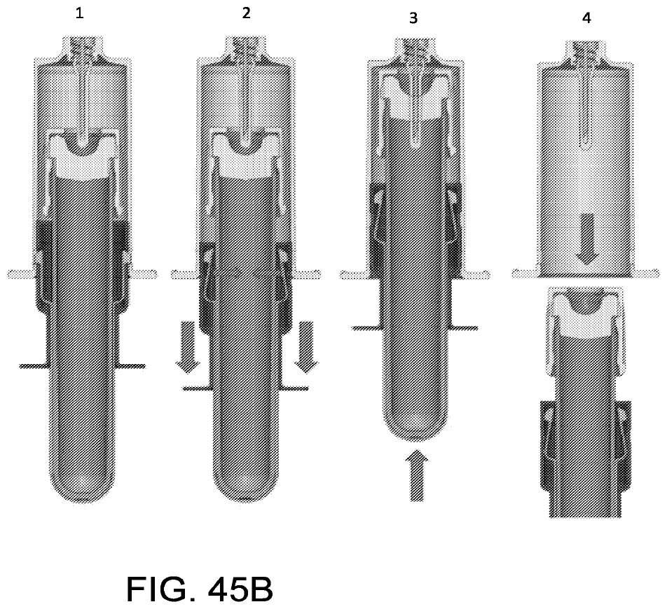

FIG. 45A-FIG. 45B is another aspect of an assembly with a securing mechanism that keeps the collection/discard tube in spaced relation from the adaptor needle during shipping and storage and will allow the collection/discard tube to advance into engagement with the needle in the adaptor for sample collection. The adaptor has two retention holes in its housing, proximal to the flange at the bottom of the adaptor. The collection/discard tube has an inner sleeve thereon. A collar is disposed on the sleeve. The collar has a first larger diameter, a second smaller diameter and a flange distal to the cap of the collection/discard tube. Disposed in the sleeve are fingers that extend outward and will fit in the retention holes in the collar when aligned therewith and holds the collection/discard tube away from the needle in the adaptor for storage and handling. Retracting the collar releases the fingers from the holes. The collection/discard tube may then be advanced upward for sample collection or downward to remove the collection/discard tube from the adaptor. After sample collection, the sample collection/discard tube is pulled from the adaptor with the sleeve and fingers affixed thereto.

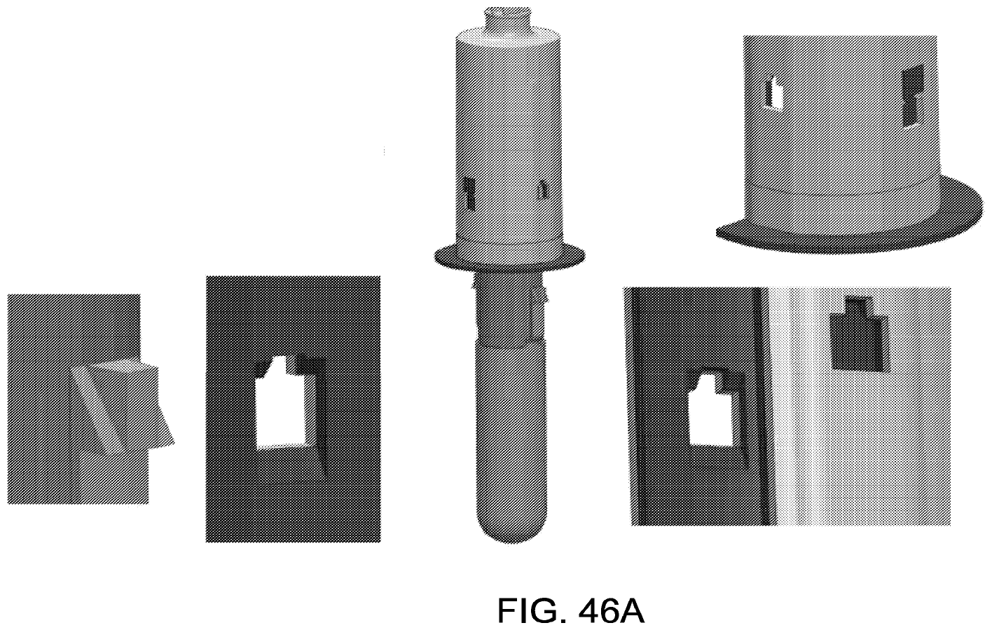

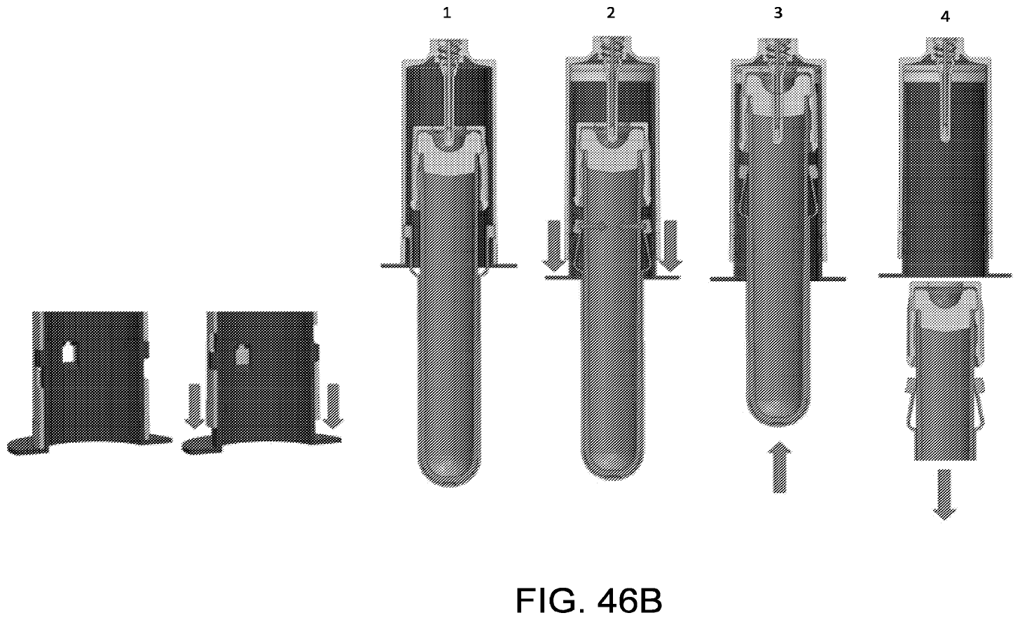

FIG. 46A-FIG. 46B is another aspect of an assembly with a securing mechanism that keeps the collection/discard tube in spaced relation from the adaptor needle during shipping and storage and will allow the collection/discard tube to advance into engagement with the needle in the adaptor for sample collection. The adaptor has two retention holes in its housing, proximal to the flange at the bottom the adaptor. The collection/discard tube has a flanged collar thereon and a holster with fingers that extend from the holster upward and outward into the adaptor. As the collar and the collection/discard tube are advanced into the adaptor, the holes in the collar align with the holes in the adaptor and the fingers extending from the holster on the collection/discard tube locks the collection/discard tube in a position away from the needle in the adaptor for storage and handling. Retracting the collar releases the fingers from the holes in the adaptor housing. The collection/discard tube is then advanced upward for sample collection. After sample collection, the sample collection/discard tube is pulled from the adaptor with the holster and fingers affixed thereto.

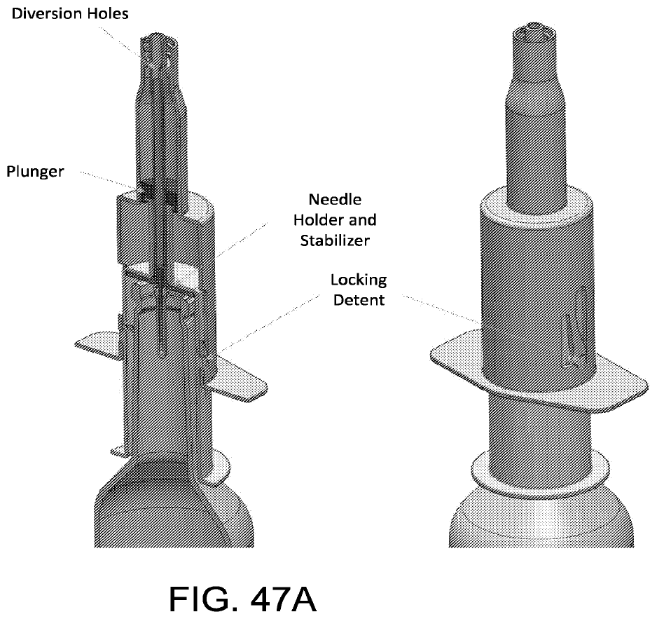

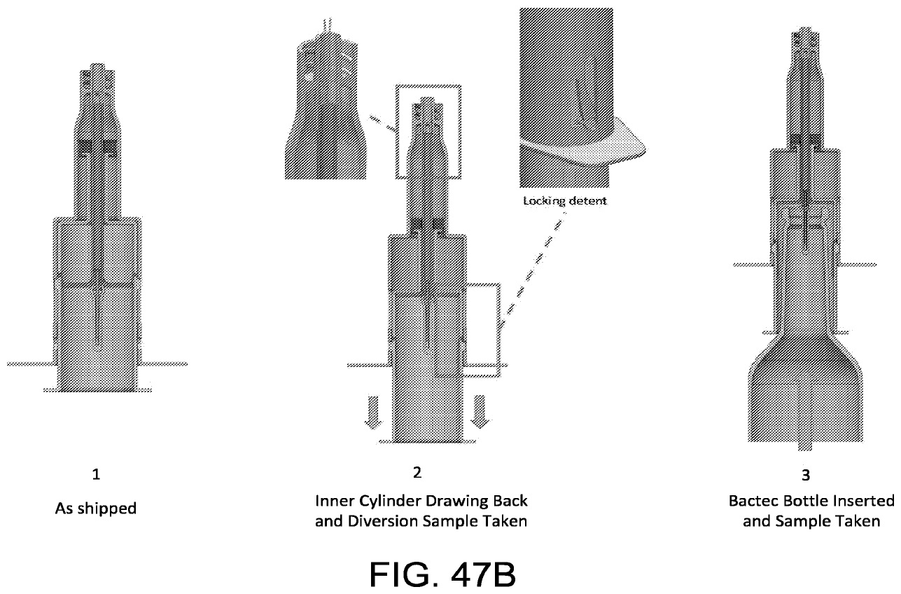

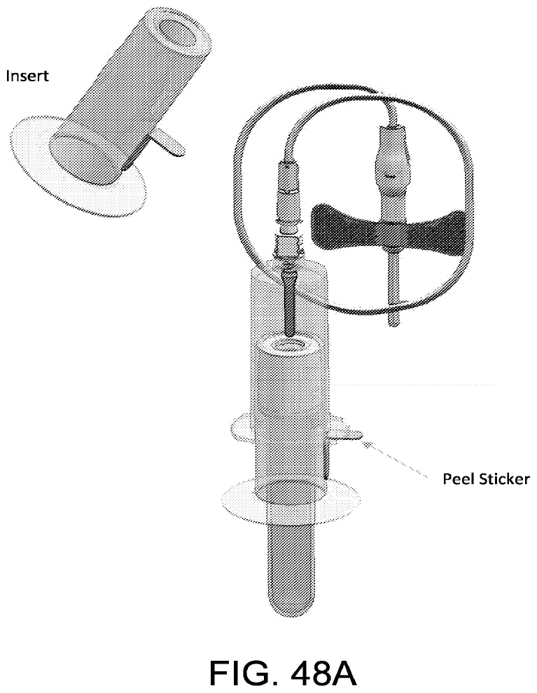

FIG. 47A-47B illustrates a diversion adaptor that provide a first flow path for collecting a first portion of the collected sample and a second flow path that collects the portion of the sample to be used for testing. The adaptor has an inner sleeve that is retracted into the adaptor when shipped. The inner sleeve is drawn back and posts in the inner sleeve lock into a detent in the adaptor housing and the diversion portion of the sample is drawn through the first flow path in the adaptor into an upper chamber of the adaptor. After the diversion sample is collected, the inner sleeve is advanced upward and the adaptor engages the sample collection bottle, and the sample travels through the second flow path into the sample collection bottle.

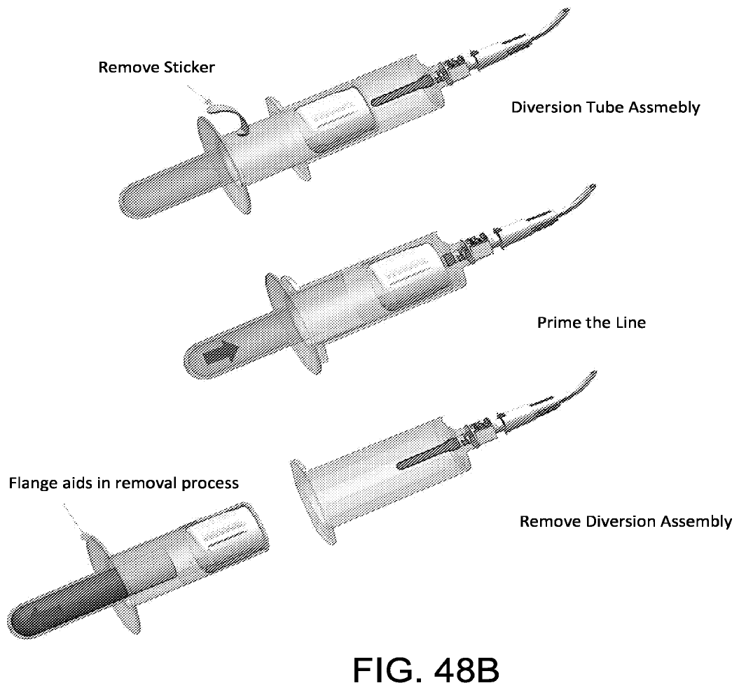

FIG. 48A-FIG. 48B illustrate another aspect of an assembly with a securing mechanism that keeps the collection/discard tube in spaced relation from the adaptor needle during shipping and storage and will allow the collection/discard tube to advance into engagement with the needle in the adaptor for sample collection. In this aspect, the collection/discard tube is delivered into the adaptor within a sleeve that has a flange on the end of the sleeve distal from the adaptor needle. In the first position, when the collection/discard tube is held away from the adaptor needle for shipment and storage, tape secures the sleeve in a position where the sleeve flange is spaced apart from the adaptor flange at the adaptor opening. When the sticker is peeled away, the collection/discard tube and the sleeve are advanced further into the adaptor for sample collection. After sample collection, the sample collection/discard tube is pulled from the adaptor with the sleeve.

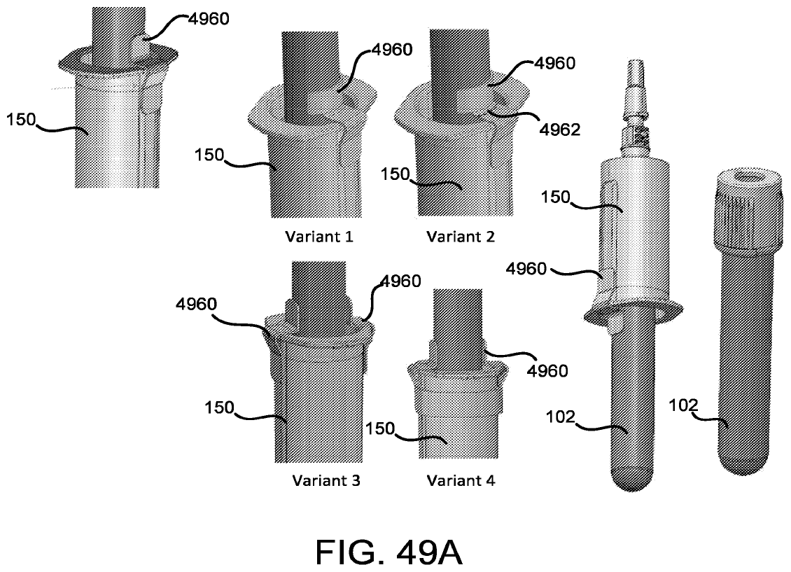

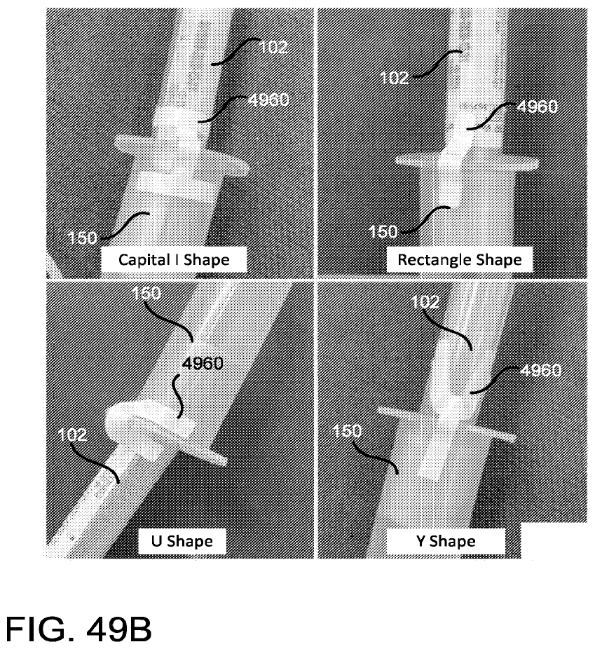

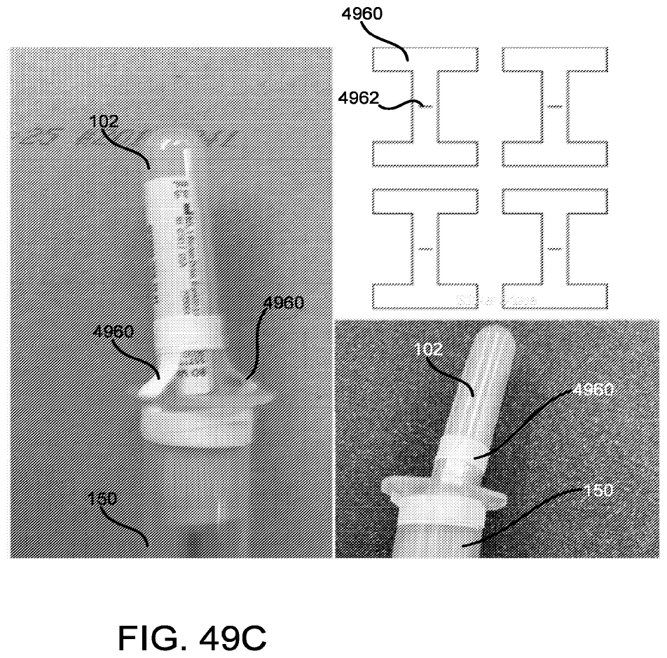

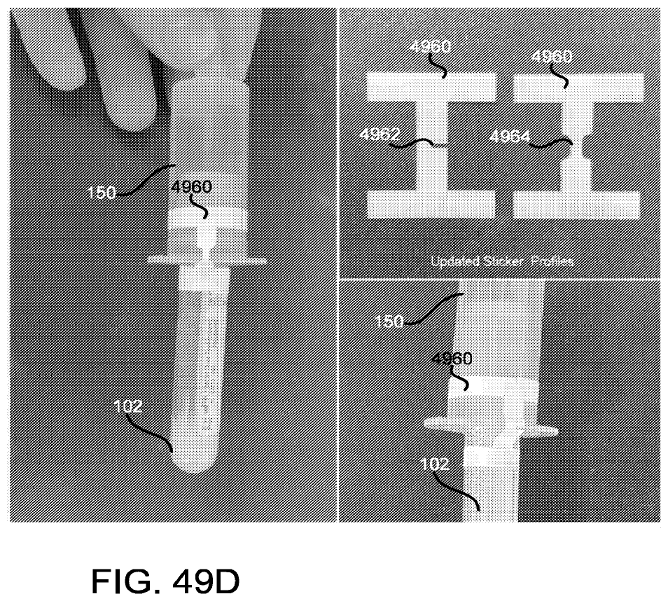

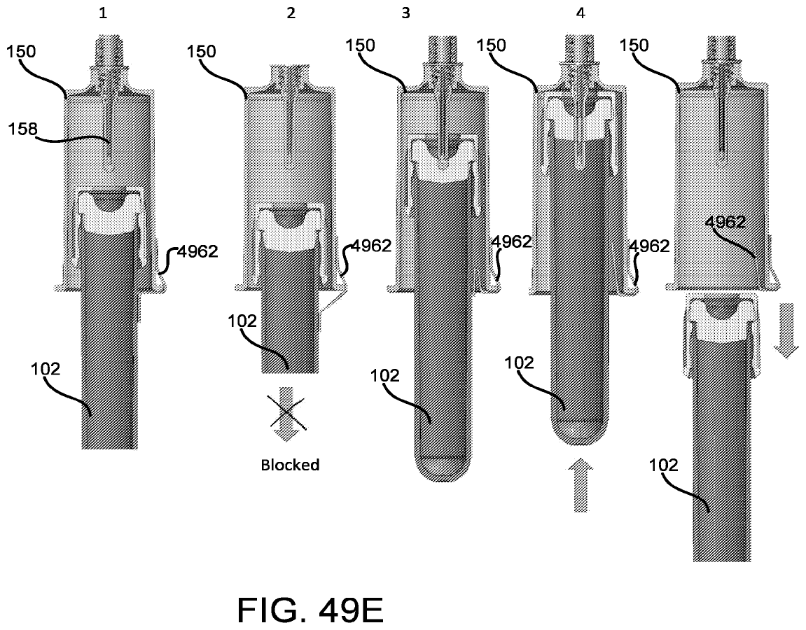

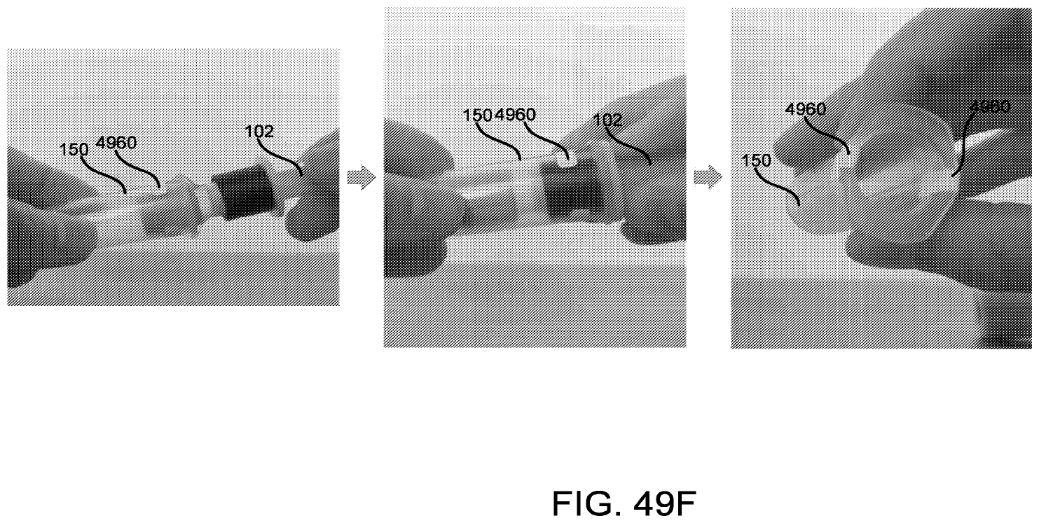

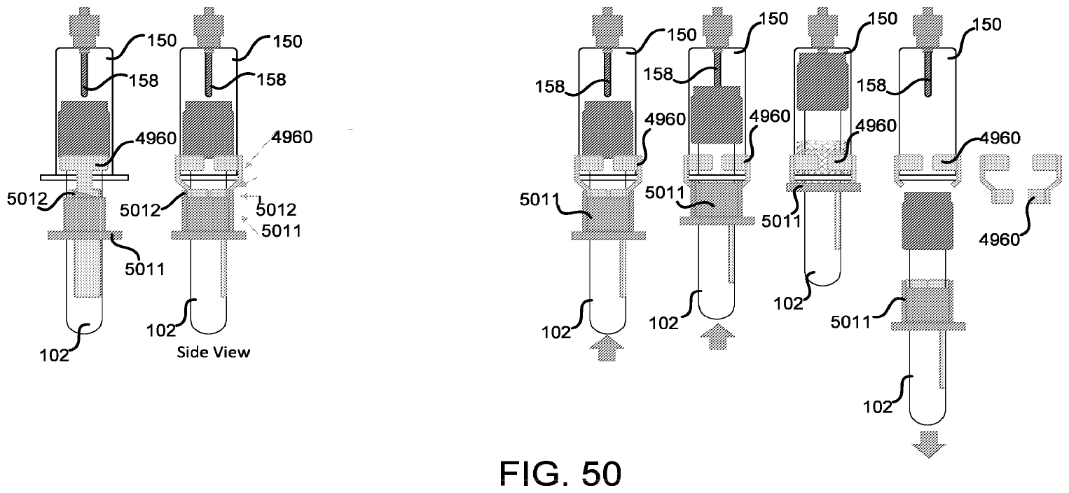

FIG. 49A-FIG. 49F illustrate different sticker configurations and placements to maintain the collection/discard tube 102 in spaced relation from the adaptor needle 158 during shipping and storage and will allow the collection/discard tube 102 to advance into engagement with the needle 158 in the adaptor 150 for sample collection upon removal of the sticker 4960. As shown, one or more stickers 4960 may be disposed over the base end of the adaptor 150 and over a portion of the exterior of the tube 102. The sticker placement and configuration are selected such that the tube 102 cannot be pulled from or pushed into the adaptor 150 inadvertently. Sticker 4960 holds the tube 102 in place prior to use. Moreover, the sticker placement and configuration are such that when the tube 102 is pushed into the adaptor 150, the portion of the sticker 4960 that is adhered to the exterior of tube 102 is peeled away, or torn or otherwise decoupled from tube 102 (as shown in FIG. 49E) to allow the collection/discard tube to advance to the second position. In one aspect, as the tube 102 is pushed into the adaptor 150, perforations 4962 (or thinned sections 4964) in the sticker are configured to tear to sever the sticker 4960 and allow the tube 102 to be pushed into engagement with the needle 158. As shown, the stickers 4960 may come in a variety of shapes (e.g., capital I, rectangle, U, Y shaped, etc.). The sticker may include perforations 4962 or thinner sections 4964 to help the sticker tear consistently. The sticker 4960 may be configured with suitable properties, such as, sticker size, orientation, peroration(s) 4962 (quantity of perforations, location of perforations, depth of perforations, etc.) such that the sticker(s) 4960 maintains the spaced relationship and peel away from the tube 102 and when the tube 102 is pushed into adaptor 150 into engagement with the needle 158. In one aspect, including one sticker on either side of the tube 102 and adaptor 150 maintains alignment of the tube 102 within the adaptor 105 with the needle 150 for engagement with the tube 102 during insertion of the tube 102 into adaptor 150.

FIG. 50 illustrates a different sticker configuration and placement to maintain the collection/discard tube 102 in spaced relation from the adaptor needle 158 during shipping and storage and will allow the collection/discard tube 102 to advance into engagement with the needle 158 in the adaptor 150 for sample collection upon removal of the sticker(s) 4960. In this aspect, the securing mechanism of the collection/discard tube 102 has a collar 5011 (e.g., including the same features as collar 510 and other similar collars described above) thereon and the sticker(s) 4960 maintains separation of the collar 5011 from the adaptor 150 and of the tube 102 from the needle 158. The collar 5011 includes cutting blade(s) 5012 configured to cut or sever the sticker(s) 4960 (e.g., in a central, perforated or thinner section thereof) into two separate pieces. With the sticker(s) 4960 severed, the collar 5011 may advance into the adaptor 150 for sample collection. After sample collection, the collection/discard tube 102 along with the collar 5011 is removed from the adaptor 150.

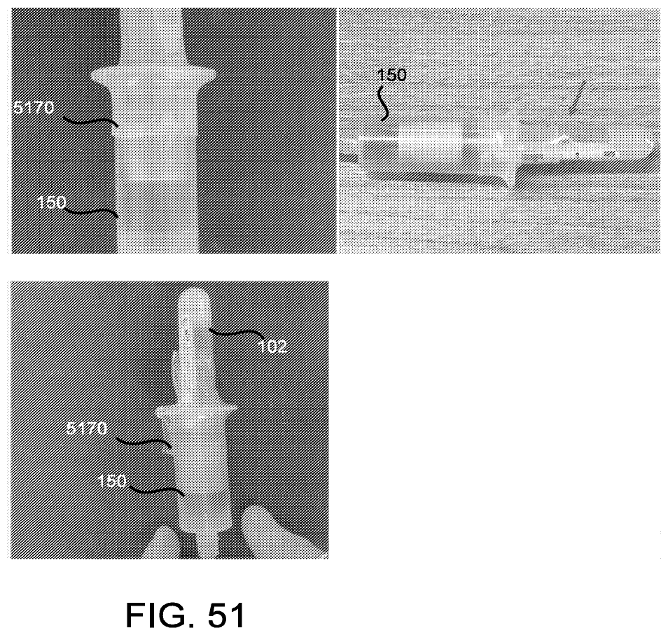

FIG. 51 illustrates the use of a heat shrink material or wrap 5170 to maintain the collection/discard tube 102 in spaced relation from the adaptor needle 158 during shipping and storage and will allow the collection/discard tube 102 to advance into engagement with the needle 158 in the adaptor 150 for sample collection upon removal of the heat shrink material 5170. To advance the collection/discard tube 102 into the adaptor 150, the heat shrink material 5170 is removed. The shrink wrap 5170 may include perforations (as described below in relation to wrap 5770) to facilitate easy tear-off/splitting the wrap 5170 and removal of the wrap 5170 to allow insertion of the tube 102 into adaptor 150. The wrap 5170 has the advantage of conforming to different geometries of the adaptor 150, tube 102, securing mechanisms, etc. that may be present in the assembly. After collection, the collection/discard tube 102 is removed from the adaptor 150.

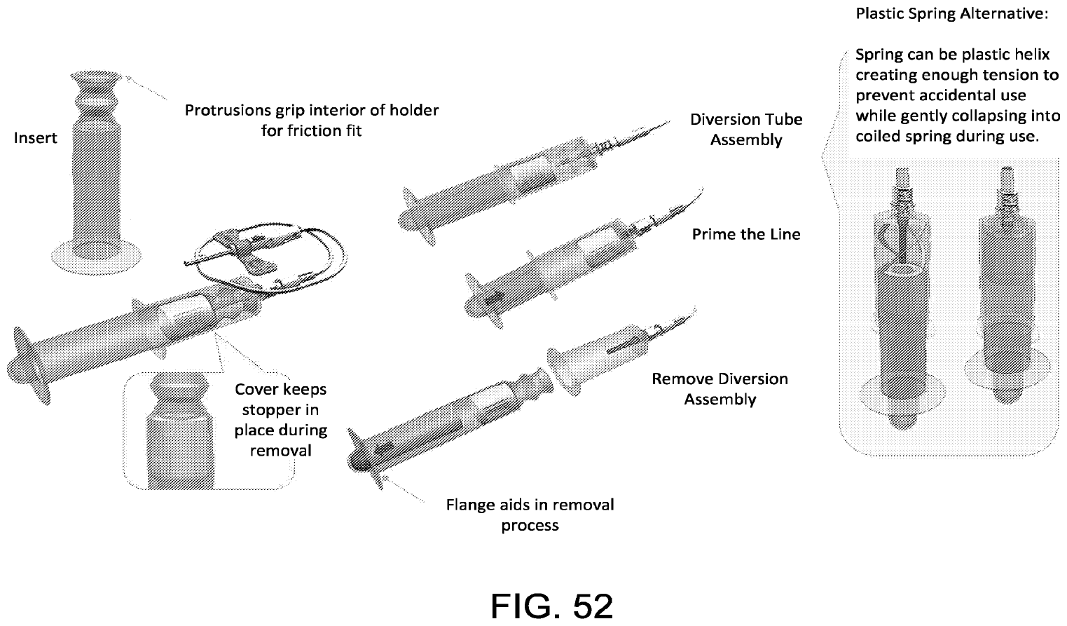

FIG. 52 illustrates another aspect of an assembly with a securing mechanism that keeps the collection/discard tube in spaced relation from the adaptor needle during shipping and storage and will allow the collection/discard tube to advance into engagement with the needle in the adaptor for sample collection. In this aspect, the collection/discard tube is delivered into the adaptor within a sleeve that has a bellows at the end thereof. The bellows operates to keep the collection/discard tube separated from the adaptor needle during shipment and storage. When sample collection is commenced, the collection/discard tube is forced upward, collapsing the bellows and allowing the adaptor needle to insert into the collection/discard tube for sample collection. When the desired amount of sample is collected, the collection/discard tube and sleeve are removed from the adaptor.

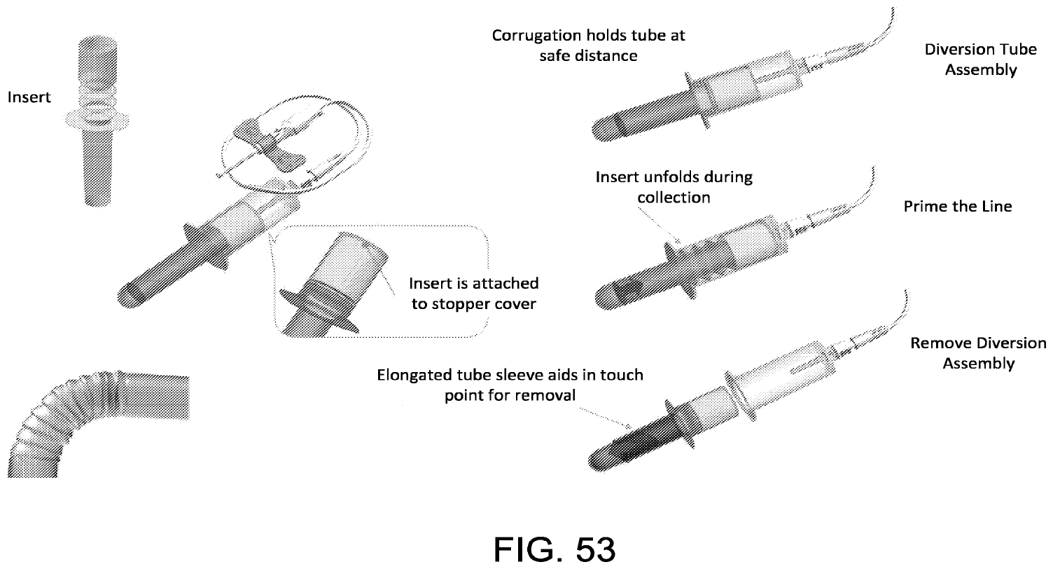

FIG. 53 illustrates another aspect of an assembly with a securing mechanism that keeps the collection/discard tube in spaced relation from the adaptor needle during shipping and storage and will allow the collection/discard tube to advance into engagement with the needle in the adaptor for sample collection. In this aspect, the collection/discard tube is delivered into the adaptor within a sleeve that has a collapsed, corrugated portion. The collapsed, corrugated portion operates to keep the collection/discard tube separated from the adaptor needle during shipment and storage. When sample collection is commenced the corrugated portion unfolds and the collection/discard tube is forced upward, allowing the adaptor needle to insert into the collection/discard tube for sample collection. When the desired amount of sample is collected, the collection/discard tube and sleeve are removed from the adaptor.

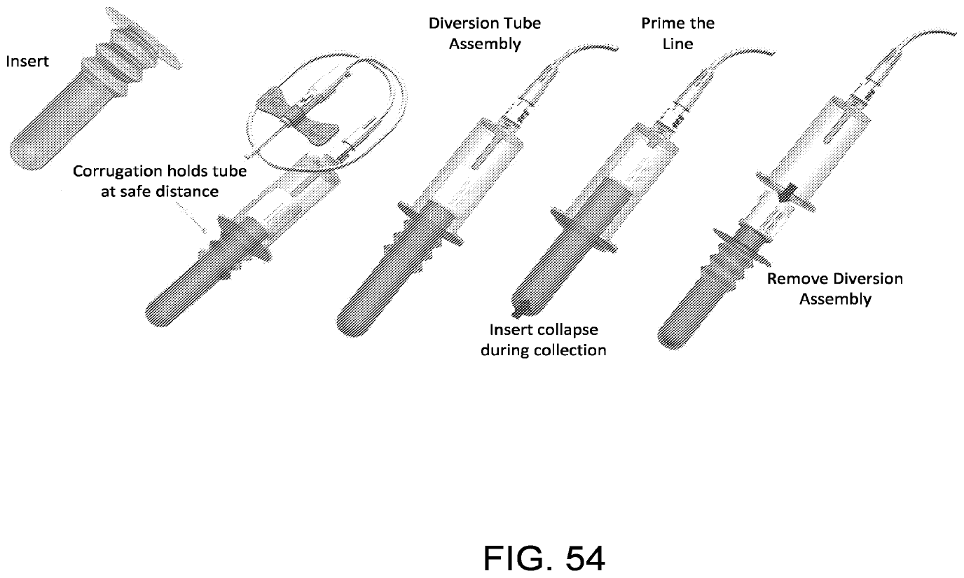

FIG. 54 is another aspect of the assembly described in FIG. 52. FIG. 54 illustrates an assembly with a securing mechanism that keeps the collection/discard tube in spaced relation from the adaptor needle during shipping and storage and will allow the collection/discard tube to advance into engagement with the needle in the adaptor for sample collection. In this aspect, the collection/discard tube is disposed in a sleeve with a bellows at the end thereof, adjacent a flange that does not permit the bellows to enter the adaptor. The bellows operates to keep the collection/discard tube separated from the adaptor needle during shipment and storage. When sample collection is commenced the collection/discard tube is forced upward, collapsing the bellows and allowing the adaptor needle to insert into the collection/discard tube for sample collection. When the desired amount of sample is collected, the collection/discard tube is removed from the adaptor. The sleeve, having never entered the adaptor, remains on the collection/discard tube.

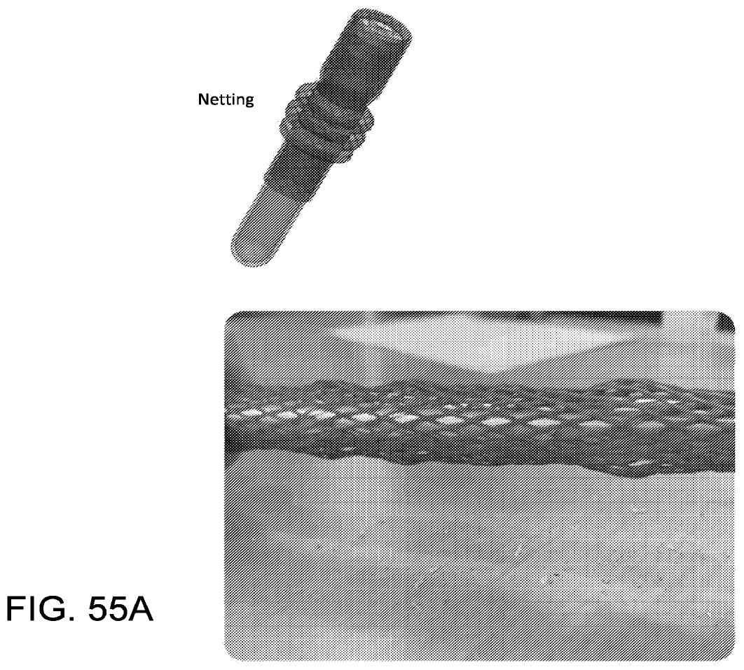

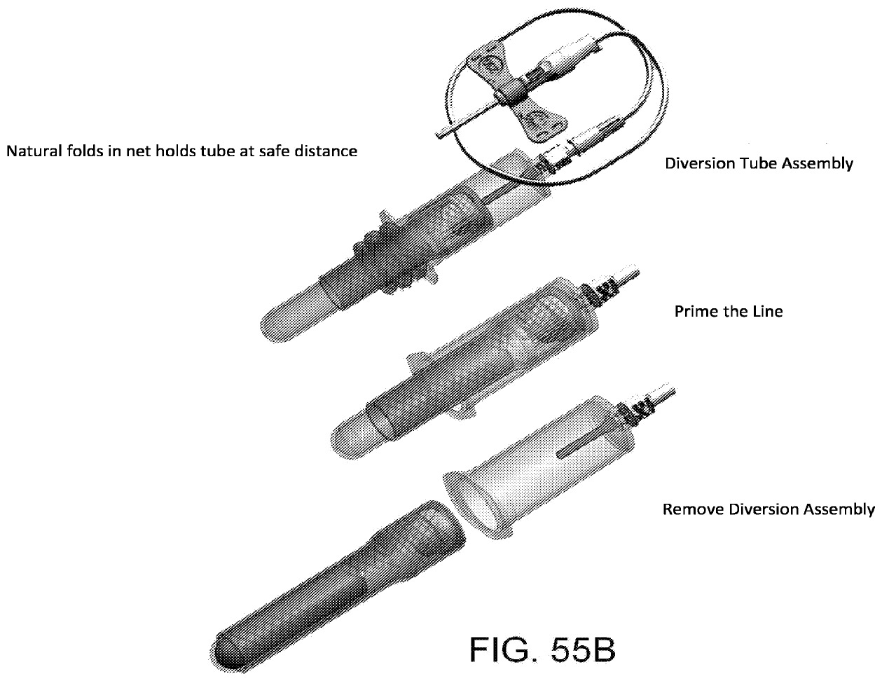

FIG. 55A-FIG. 55B is another aspect of an assembly with a securing mechanism that keeps the collection/discard tube in spaced relation from the adaptor needle during shipping and storage and will allow the collection/discard tube to advance into engagement with the needle in the adaptor for sample collection. In this aspect, the collection/discard tube is placed in a sleeve with netting disposed on the exterior of the collection/discard tube. Folds in the netting operate to keep the collection/discard tube separated from the adaptor needle during shipment and storage. When sample collection is commenced the collection/discard tube is forced upward, allowing the folds to unfold, thereby allowing the collection/discard tube to engage the adaptor needle, whereby the needle is inserted into the collection/discard tube for sample collection. When the desired amount of sample is collected, the collection/discard tube is removed from the adaptor along with the netting.

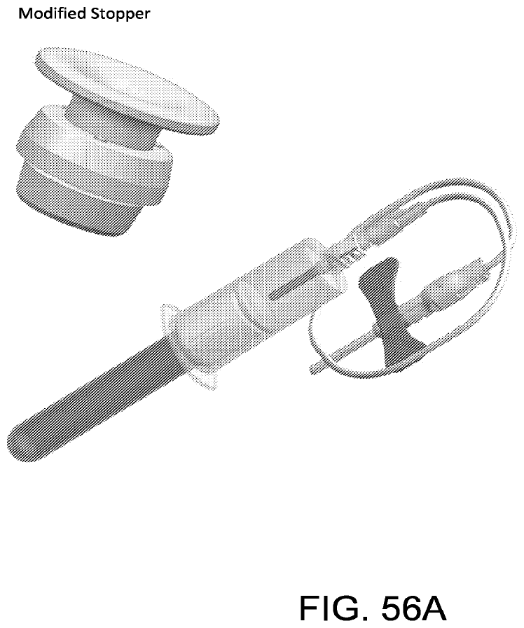

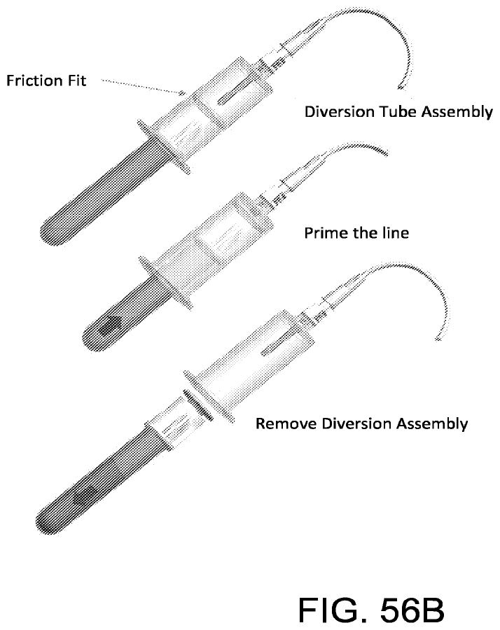

FIG. 56 is another aspect of an assembly with a securing mechanism that keeps the collection/discard tube in spaced relation from the adaptor needle during shipping and storage and will allow the collection/discard tube to advance into engagement with the needle in the adaptor for sample collection. In this aspect, a stopper is placed on top of the cap of the collection/discard tube. The stopper has a perimeter that forms a friction fit with the adaptor when the stopper/collection/discard tube assembly is placed within the adaptor. The friction fit allows the stopper to maintain the collection/discard tube in spaced relation with the adaptor needle.

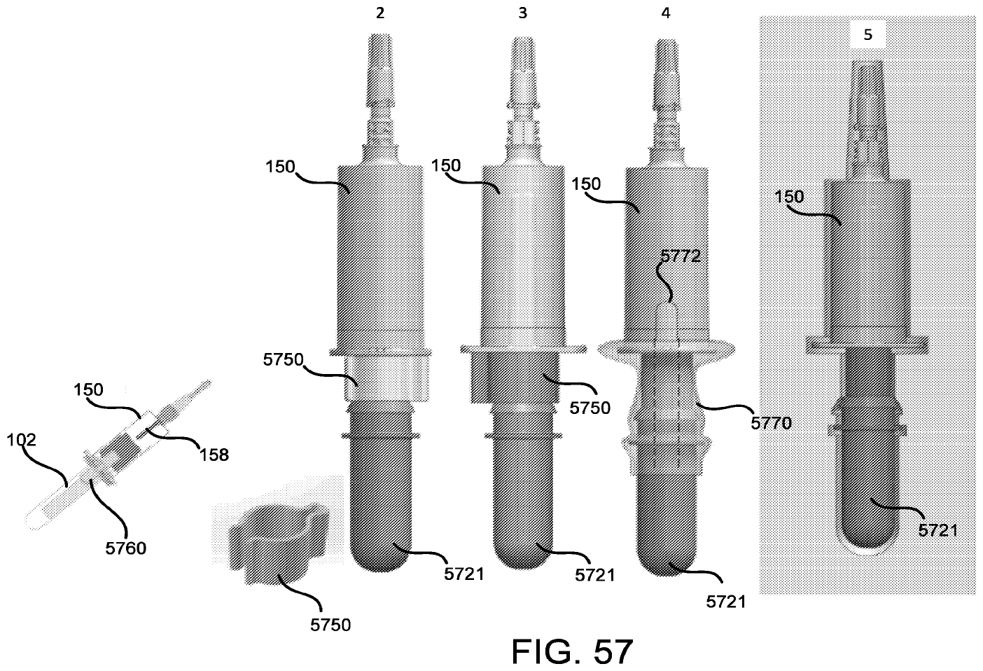

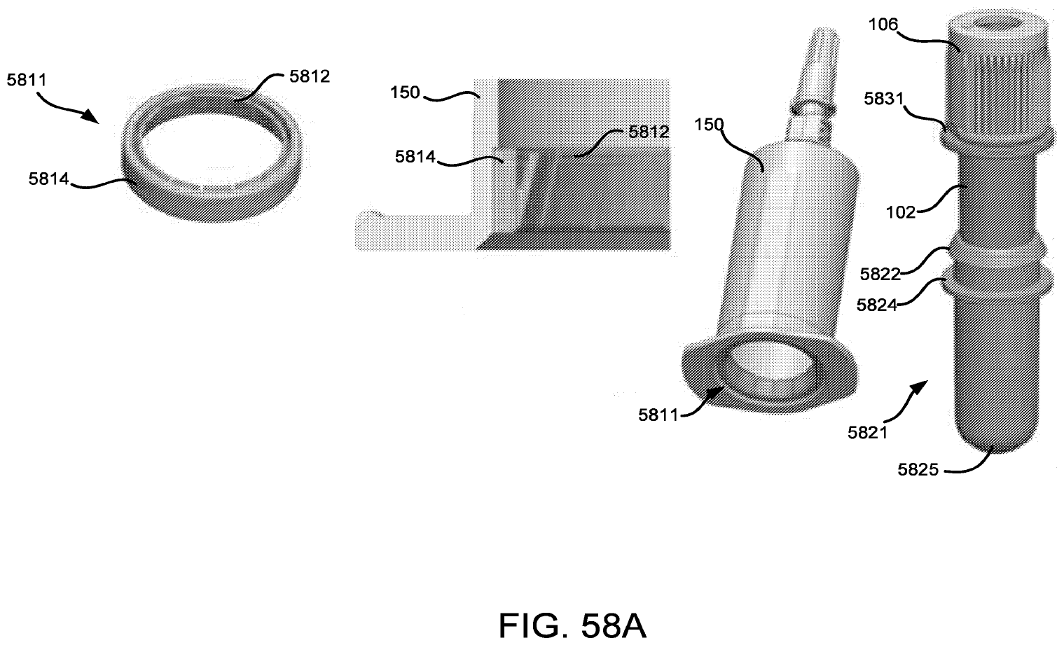

FIG. 57 is another aspect of an assembly with a clamp-like or shrink wrap securing mechanism that keeps the collection/discard tube 102 in spaced relation from the adaptor needle 158 during shipping and storage and will allow the collection/discard tube 102 to advance into engagement with the needle 158 in the adaptor 150 for sample collection. In this aspect, a removeable clip 5750, tear-off strip or sticker 5760, or a shrink wrap 5770 (disposed over the base end of adaptor 150 and over tube 102 and at least a portion of sleeve 5721) is used to force a spaced relationship between a sleeve 5721 (configured with the same features as sleeve 5821 described below) in which the collection/discard tube 102 is disposed and the adaptor 150. The shrink wrap 5770 may include a tear handle 5772 and perforations (indicated by dotted lines) for tearing the shrink wrap 5770 off of the assembly. It is to be appreciated that the assembly may include any of the securing mechanisms described herein and the clip, sticker, or shrink wrap may be used in addition to or instead of the securing mechanism to maintain the spaced relationship. The spaced relationship ensures that the collection/discard tube 102 does not engage the adaptor needle 158 during storage and shipping. When sample collection commences, the clip 5750 or sticker 5760 is removed and the sleeve in which the collection/discard tube is disposed can be advanced into contact with the adaptor needle 158 for sample collection. After sample collection, the collection/discard tube 102 (still disposed in the sleeve) is removed from the adaptor.

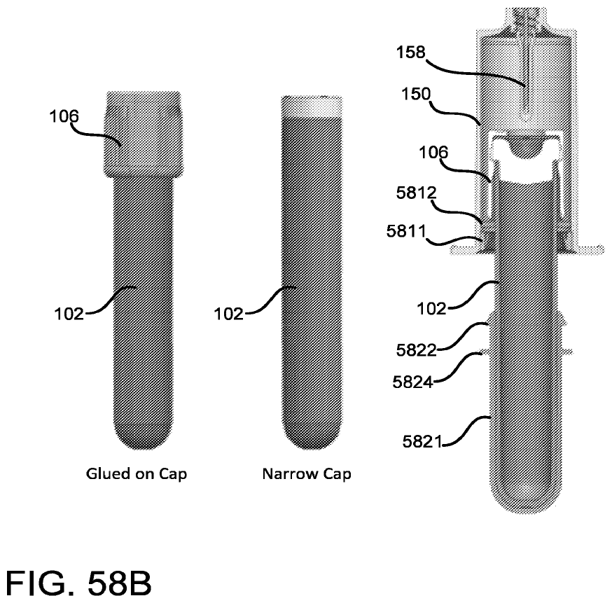

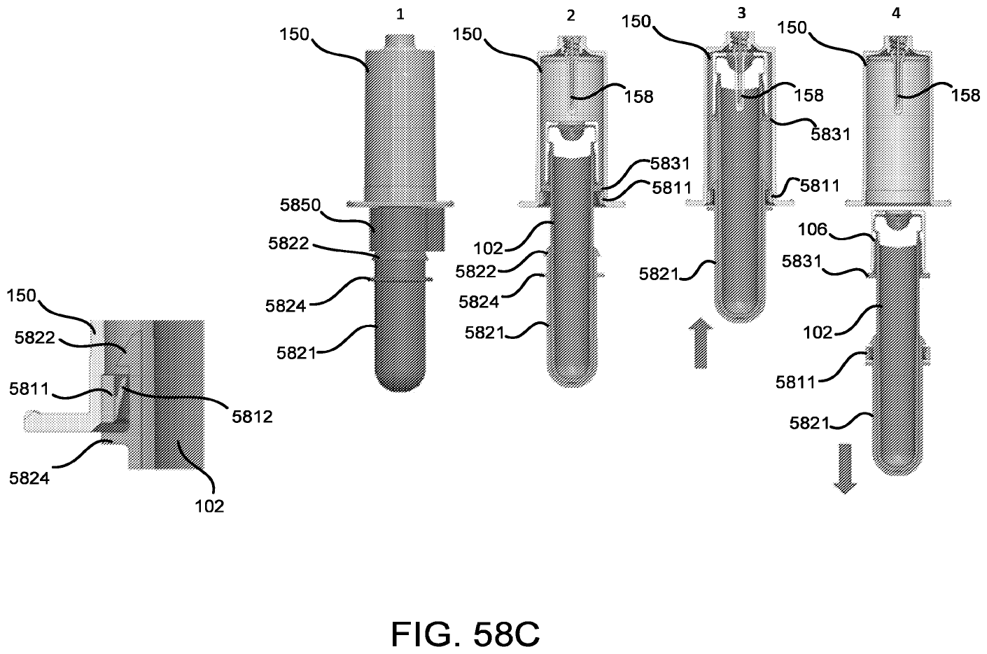

FIGS. 58A-58C is an alternative aspect of FIG. 17A-FIG. 17B, FIG. 18A-FIG. 18C, and FIG. 19A-FIG. 19C. Illustrated is a securing mechanism comprising a three-piece assembly for holding the collection/discard tube 102 in spaced relation from the needle 158 in the adaptor 150 during shipment but allowing the collection/discard tube 102 to be advanced into engagement with the needle 158 for sample collection. The three-piece assembly is pulled from the adaptor 150 when the collection/discard tube 102 is removed therefrom. The securing mechanism comprises snap ring 5811, sleeve 5821, and ring 5831. The snap ring 5821 includes upward facing projections 5812 extending for a ring base 5814 and biased to be angled toward the interior of snap ring 5811. The outer diameter of ring base 5814 is fitted to be snugly retained in the base open base end of the adaptor 150. The sleeve 5821 in this aspect is configured in tubular shape with an open end that receives a portion of tube 102 and a closed end 5825. In one aspect, sleeve 5821 is fixed (e.g., via adhesive) to the tube 102. The sleeve 5821 includes first and second flanges or raised portions 5822 and 5824. The collar or ring 5831 is placed adjacent the cap 5831 on the collection discard tube 102. The collar 5831 is dimensioned to be advanced through and above the snap ring 5811 and permitted to pass the projections 5812. The outer diameter of the collar 5831 may be configured to grip the interior of adaptor 150 to hold the collection/discard tube 102 spaced apart from the adaptor needle 158 during shipment and storage. Additionally and/or alternatively, the projections 5812 may grip the exterior of the tube 102 to hold the tube 102. In one aspect, the adaptor 150 includes a stepped end in the base to receive the snap ring 5811. The collar 5831 abuts against the ends of projections 5812 and thus tube 102 is prevented from being pulled out of adaptor 150 easily and is held in the spaced relationship with needle 158. The tube 102 may be pushed into the adaptor 150 further with the projections 5812 permitting raised portion 5822 to pass through snap ring 5811 and the projections snap to the exterior of sleeve 5821 to the area between raised portions 5822 and 5824. In this position, the snap ring 5811 and the sleeve 5831 are locked to each other and projections 5812 prevent the tube 102 from being pulled easily out of the adaptor 150. Moreover, in this position, the collection tube 102 is engaged with the needle 158 for sample collection. The first flange 5822 on the sleeve 5821 and catches on the ends of projections 5812 when the tube 102 is pulled out of the adaptor 150 to pull the snap ring 5811 from the adaptor 150 when the collection/discard tube 102 and sleeve 5821 are removed therefrom. In one aspect, as shown in FIG. 58C, when the assembly is stored or shipped, a removable clip 5850 (e.g., configured with the same features as clip 5750) is disposed around tube 102 between the open end of sleeve 5821 and the collar 5831 that abuts against the base of adaptor 150 and the sleeve 5831. The clip 5850 prevents the tube 102 from being pushed prematurely into adaptor 150 and the ring 5831 that abuts against the snap ring 5811 prevents the tube 102 from being pulled from adaptor 150. Thus, the tube 102 is held in the spaced relationship from needle 158.

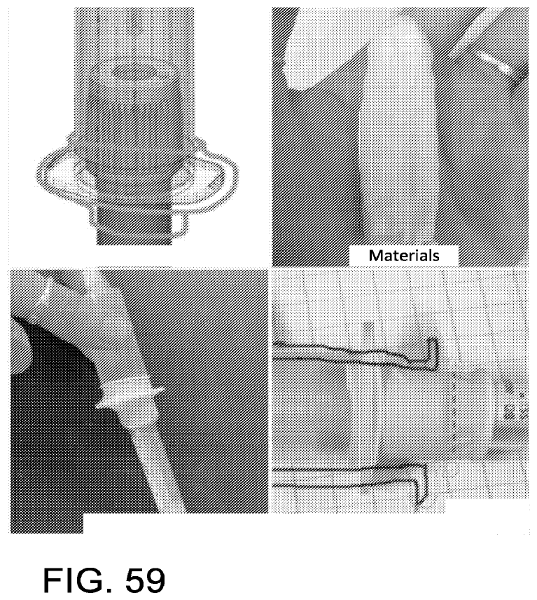

FIG. 59 is a proof of concept illustration of using an elastomeric tube to hold the collection/discard tube in spaced relation from the needle in the adaptor during shipment but allowing the collection/discard tube to be advanced into engagement with the needle for sample collection. When the elastomer tube is removed, the collection/discard tube may be brought into engagement with adaptor needle for sample collection. When the desired amount of sample is collected, the collection/discard tube is removed from the adaptor.

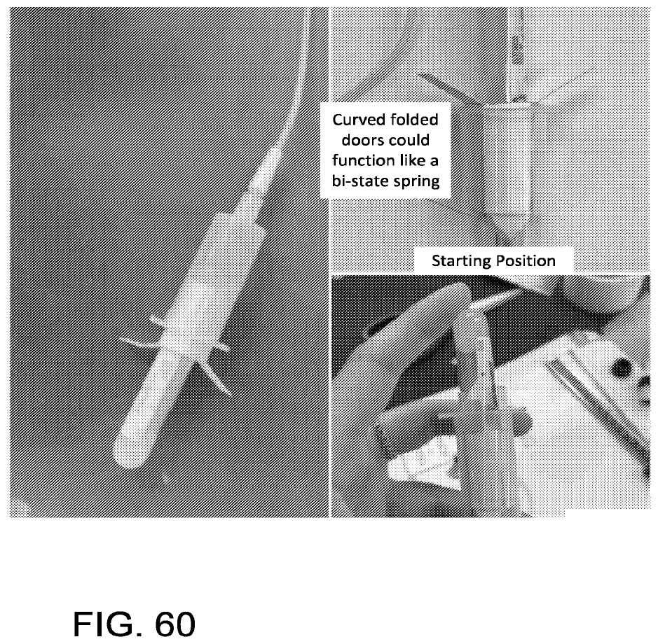

FIG. 60 illustrates extensions (e.g., made of any suitable material) from a sleeve on a collection/discard tube to hold the collection/discard tube in spaced relation from the needle in the adaptor during shipment but allowing the collection/discard tube to be advanced into engagement with the needle for sample collection. When the collection/discard tube is advanced into engagement with adaptor needle for sample collection, the extensions are carried into the adaptor. When the collection/discard tube is removed from the adaptor, the extensions are carried with it.

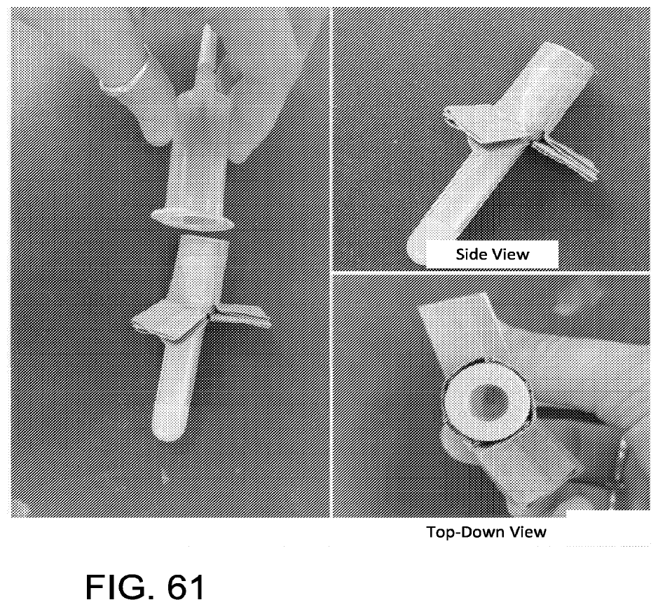

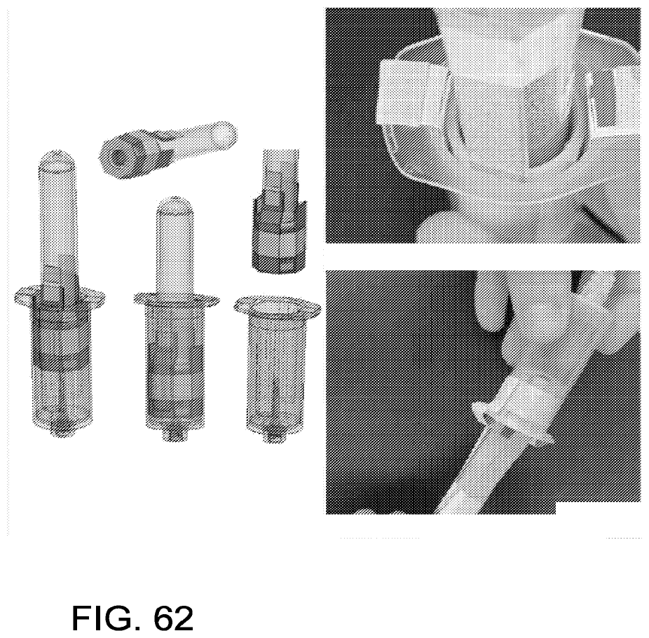

FIG. 61 is a further modification of the aspect illustrated in FIG. 60 and also is a proof of concept illustration that uses extensions from a sleeve on a collection/discard tube to hold the collection/discard tube in spaced relation from the needle in the adaptor during shipment but allowing the collection/discard tube to be advanced into engagement with the needle for sample collection. Tape is used to hold the sleeve together. In some aspects, the cardboard is corrugated for additional thickness and sturdiness. When the collection/discard tube is advanced into engagement with the adaptor needle for sample collection, the extensions are carried into the adaptor. When the collection/discard tube is removed from the adaptor, the extensions are carried with it.

FIG. 62 is a further modification of the aspect illustrated in FIG. 60 and also is a proof of concept illustration that uses extensions from a sleeve on a collection/discard tube to hold the collection/discard tube in spaced relation from the needle in the adaptor during shipment but allowing the collection/discard tube to be advanced into engagement with the needle for sample collection. Tape is used to hold the sleeve together and to hold the collection/discard tube in space relation to the adaptor needle. In some aspects, the cardboard is corrugated for additional thickness and sturdiness. When collection is commenced, the tape holding the collection/discard tube in place is removed, allowing the collection/discard tube to be advanced into engagement with the adaptor for sample collection, the extensions are carried into the adaptor. When the collection/discard tube is removed from the adaptor, the extensions are carried with it.

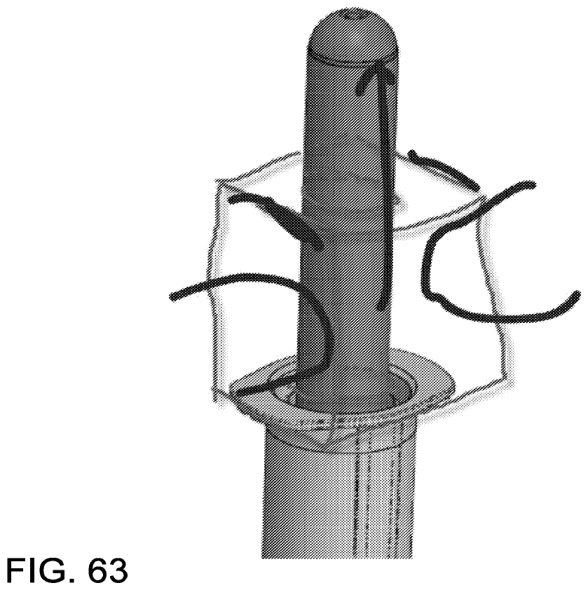

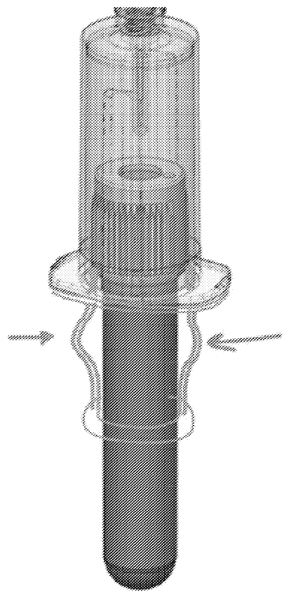

FIG. 63 is a concept illustration of a sleeve structure for the collection/discard tube that is sufficiently wide so as not to enter the adaptor, thereby keeping the collection/discard tube in spaced relation from the needle in the adaptor during shipment but allowing the collection/discard tube to be advanced into engagement with the needle for sample collection. The collection/discard tube can be advanced in the sleeve structure. After sample collection, the structure is squeezed so that the collection/discard tube assembled to the sleeve may be backed out of the adaptor.

FIG. 64 is a concept illustration of a sleeve structure for the collection/discard tube that is sufficiently wide so as not to enter the adaptor, thereby keeping the collection/discard tube in spaced relation from the needle in the adaptor during shipment but allowing the collection/discard tube to be advanced into engagement with the needle for sample collection when the sleeve is squeezed. The collection/discard tube may then be advanced in the sleeve structure. After sample collection, the structure is squeezed for the collection/discard tube assembled to the sleeve to be backed out of the adaptor.

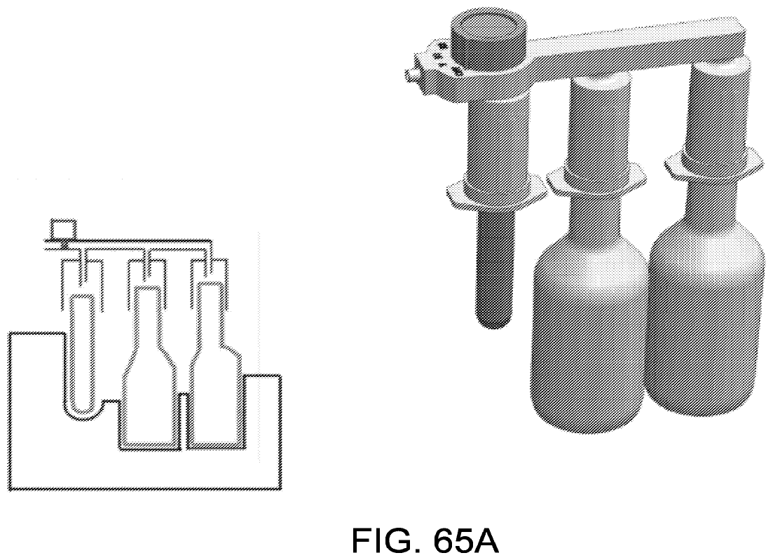

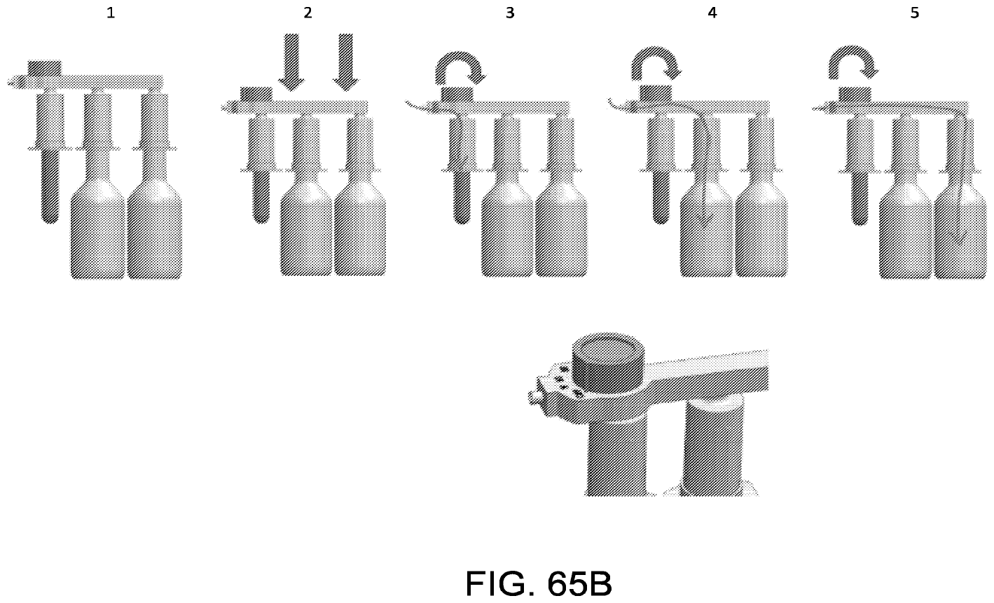

FIG. 65A-FIG. 65B is an assembly with a valve that has a first adaptor for receiving a collection/discard tube and at least one other adaptor for receiving a sample bottle. The assembly has an inlet that may be connected to a line set for collecting a sample from a patient. The valve may direct a first portion of the collected sample into the collection/discard tube. After the desired amount of sample is collected, the valve is rotated, and additional sample is directed to the one or more sample containers.

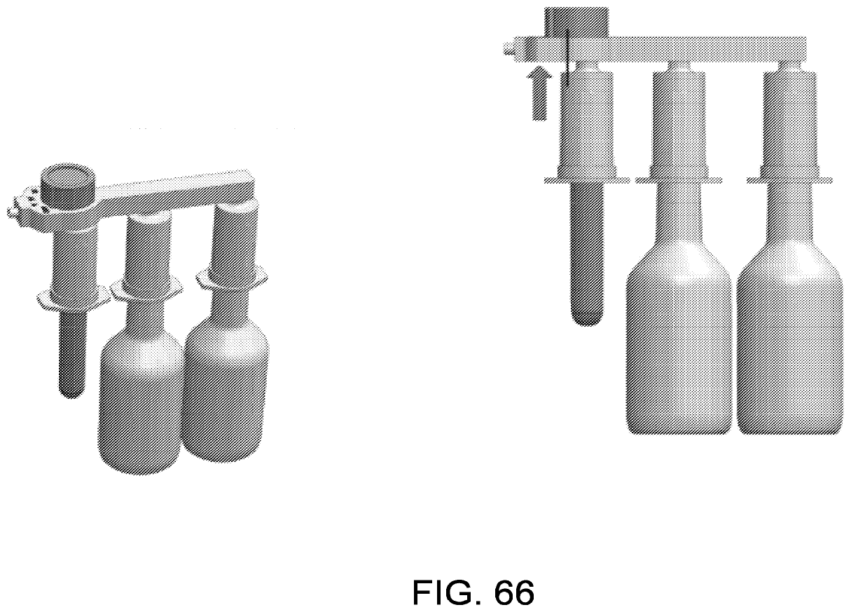

FIG. 66 is an alternative aspect of what is described in FIG. 65A-FIG. 65B. In this aspect, a pin is provided to prevent the valve from rotating beyond the position that will direct sample beyond the sample collection/discard tube adaptor unless the collection/discard tube is in engagement with the needle in the adaptor.

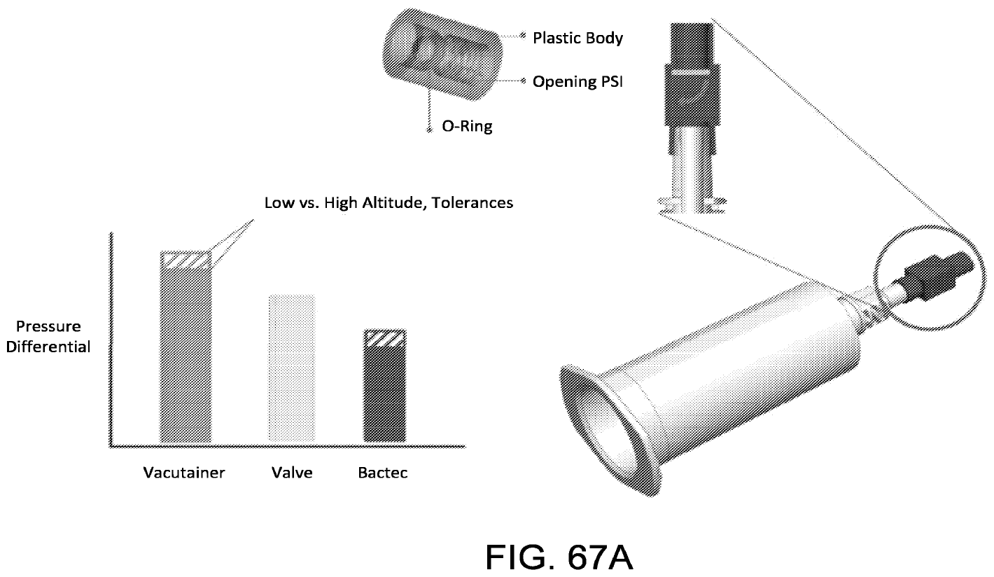

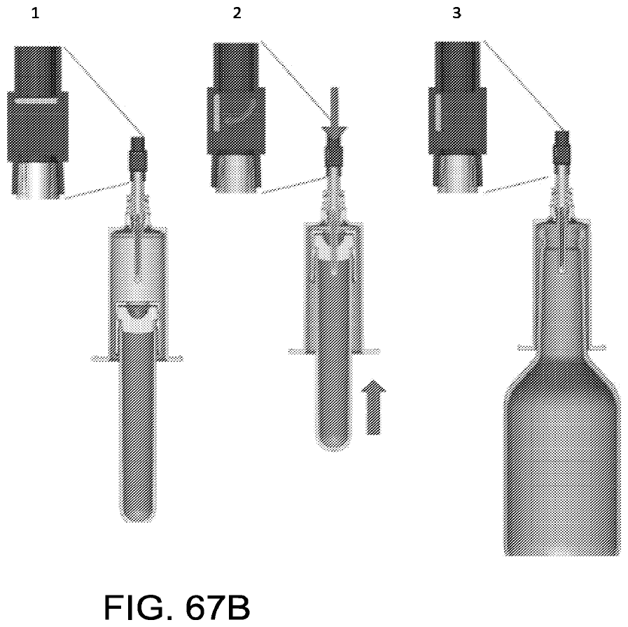

FIG. 67A-FIG. 67B illustrates an alternative aspect of the assembly described herein that deploys a collection/discard tube in communication with an adaptor for connection to a patient line set to collect sample from a patient into the container. In this aspect, the collection/discard tube is assembled with the adaptor but held in spaced relation from the adaptor needle during shipment and storage. The collection/discard tube may have one of the numerous examples of collars or sleeves or tape described herein for maintaining such spaced relation between the collection/discard tube and the needle. The adaptor has a valve on the end thereof that is placed in connection with the patient line set (not shown). The collection/discard tube is advanced into the adaptor to collect sample, after which the valve is opened. Once the initial portion of the patient sample is collected in the collection/discard tube, the collection/discard tube is removed from the adaptor and then the patient sample collection bottle is placed in fluid communication with adaptor needle for collecting the patient sample into the blood culture container.

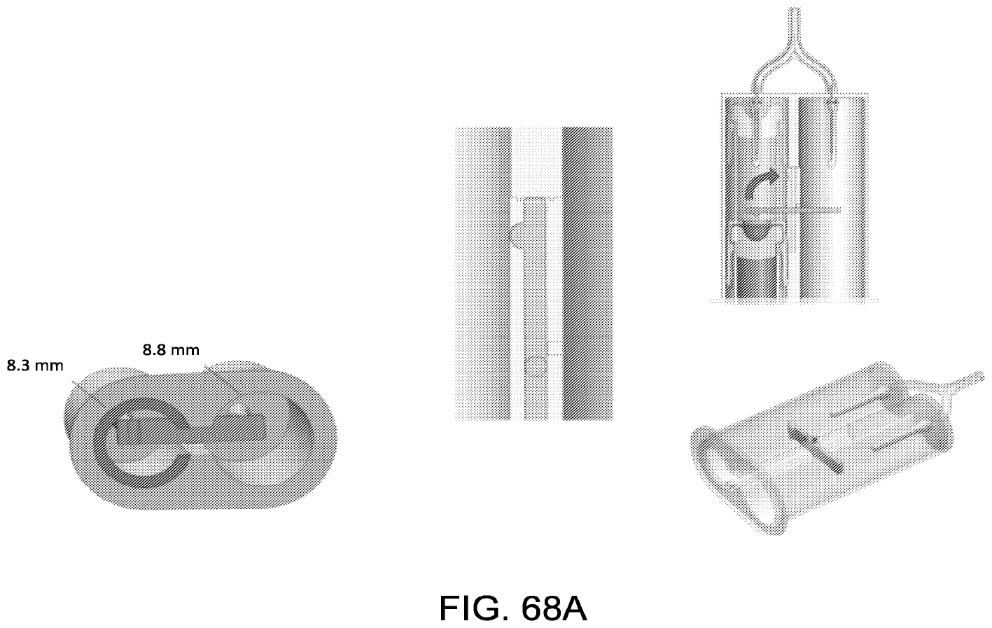

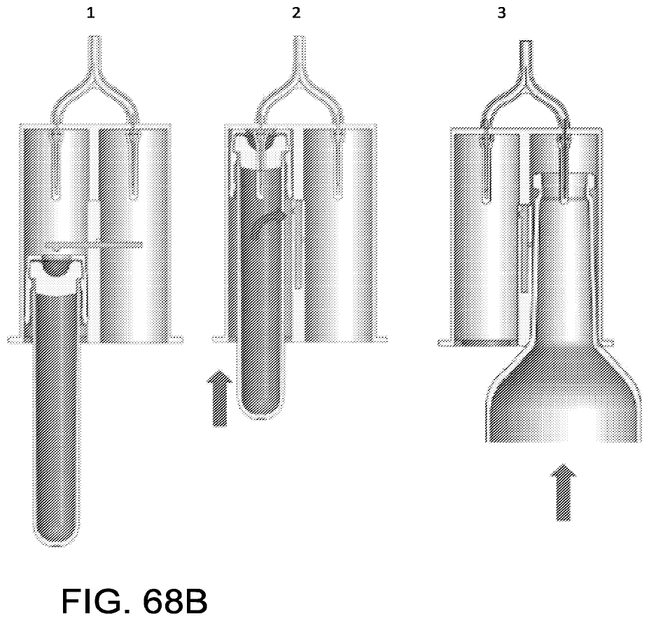

FIG. 68A-68B illustrates an alternative aspect of the assembly described herein that deploys a collection/discard tube in communication with an adaptor for connection to a patient line set to collect sample from a patient into the container. In this aspect, the collection/discard tube is assembled with the adaptor but held in spaced relation from the adaptor needle during shipment and storage by a rotating flap. The adaptor has a first port for receiving the collection/discard tube and a second port for receiving the blood culture bottle. The flap moves from a first position that is intermediate between the collection/discard tube and the adaptor needle, thereby preventing the collection/discard tube from coming into contact with the adaptor needle during shipment and storage, to a second position in which the flap is positioned alongside the collection/discard tube, and therefore no longer in the path between the collection/discard tube and the adaptor needle. Advancing the collection/discard tube in the adaptor moves the flap from the first position to the second position. The collection/discard tube is advanced into the adaptor to collect sample, after which the valve is opened. Once the initial portion of the patient sample is collected in the collection/discard tube, the collection/discard tube is removed from the adaptor and then the patient sample collection bottle is placed in fluid communication with the adaptor needle in the larger port for collecting the patient sample into the blood culture container.

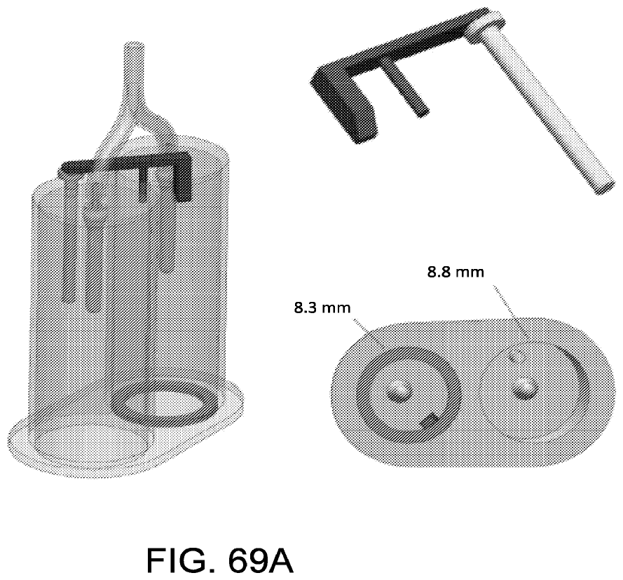

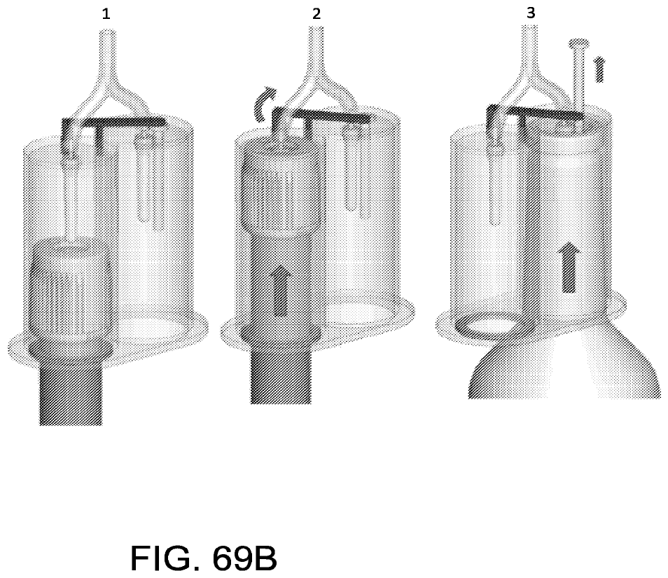

FIG. 69A-FIG. 69B illustrates an alternative aspect of the dual port adaptor described in FIG. 68A-FIG. 68B. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a lever/pin assembly with a first position and a second position. In the first position, the lever in the port that receives the collection/discard tube is positioned over a pin in the second port. In this position, the pin cannot move upward, the culture bottle cannot be brought into contact with the needle in the second port. When the collection/discard tube is advanced into fluid communication with the needle in the adaptor, the lever moves away from above the pin and the pin can be move upward, allowing the culture bottle to be placed in fluid communication with the needle in the second port for collection of the remainder of the sample. Once the initial portion of the patient sample is collected in the collection/discard tube, the collection/discard tube is removed from the adaptor and then the patient sample collection bottle is placed in fluid communication with adaptor needle in the larger port for collecting the patient sample into the blood culture container.

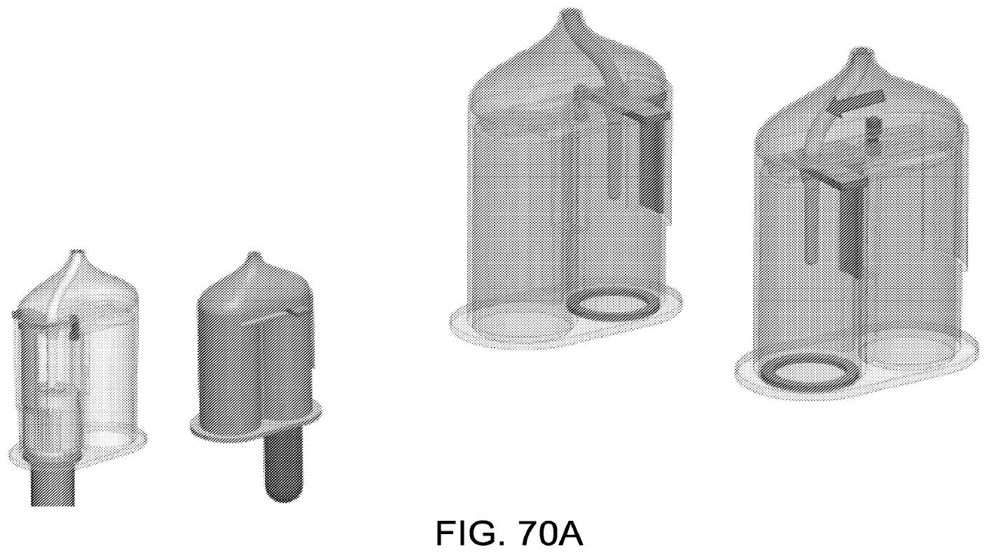

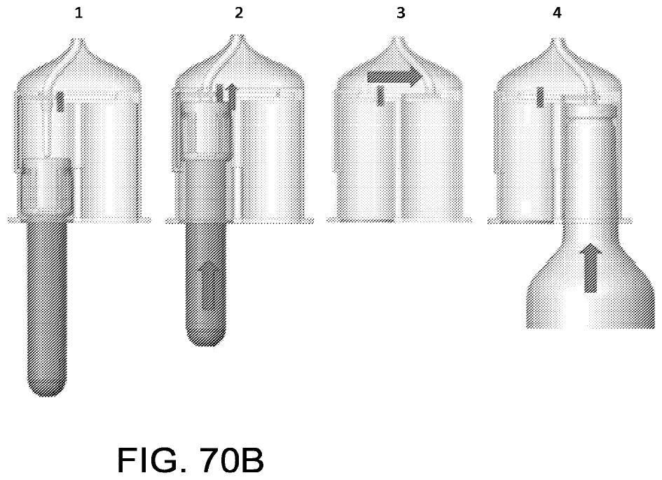

FIG. 70A-FIG. 70B illustrates an alternative aspect of the dual port adaptor described in FIG. 69A-FIG. 69B. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a needle shuttle that will move the needle from the first port to the second port. A lock out peg prevents the needle from being moved from the first port to the second port until sample is collected into the collection/discard tube in the first port. When the collection/discard tube is advanced into fluid communication with the needle in the adaptor, the lock out peg is moved upward, which allows the needle shuttle to be moved. Moving the needle shuttle moves the needle from the first port to the second port. The culture bottle can then be placed in fluid communication with the needle in the second port for collecting the remainder of the sample. Once the initial portion of the patient sample is collected in the collection/discard tube, the collection/discard tube is removed from the adaptor and then the patient sample collection bottle is placed in fluid communication with the adaptor needle in the larger port for collecting the patient sample into the blood culture container.

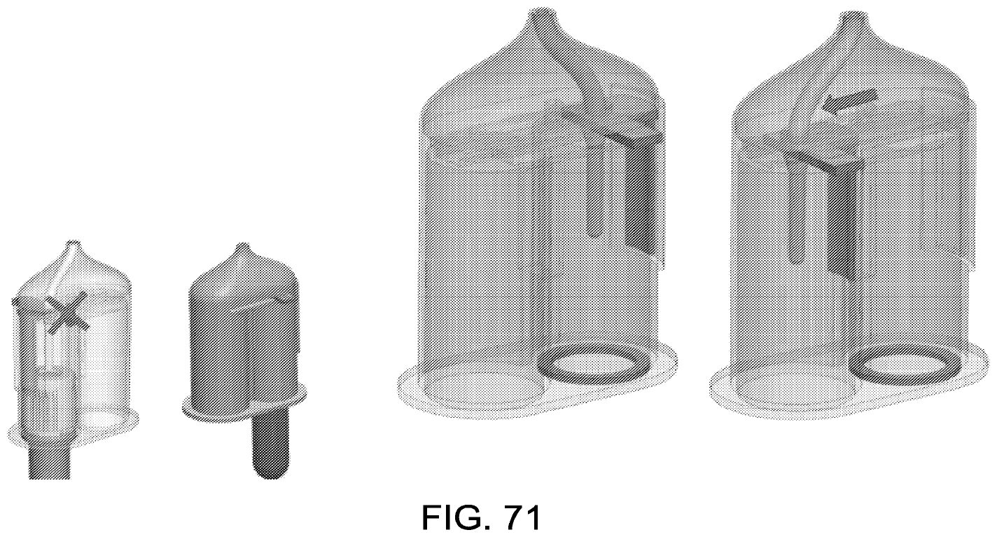

FIG. 71 illustrates an alternative aspect of the dual port adaptor described in FIG. 70A-FIG. 70B. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a needle shuttle that will move the needle from the first port to the second port. In this aspect, the assembly lacks the lock out peg. Once the initial portion of the patient sample is collected in the collection/discard tube, the collection/discard tube is removed from the adaptor, which allows the needle shuttle to be moved from the first port to the second port. and then the patient sample collection bottle is placed in fluid communication with the adaptor needle in the larger port for collecting the patient sample into the blood culture container. Moving the needle shuttle moves the needle from the first port to the second port. The culture bottle can then be placed in fluid communication with the needle in the second port for collection the remainder of the sample.

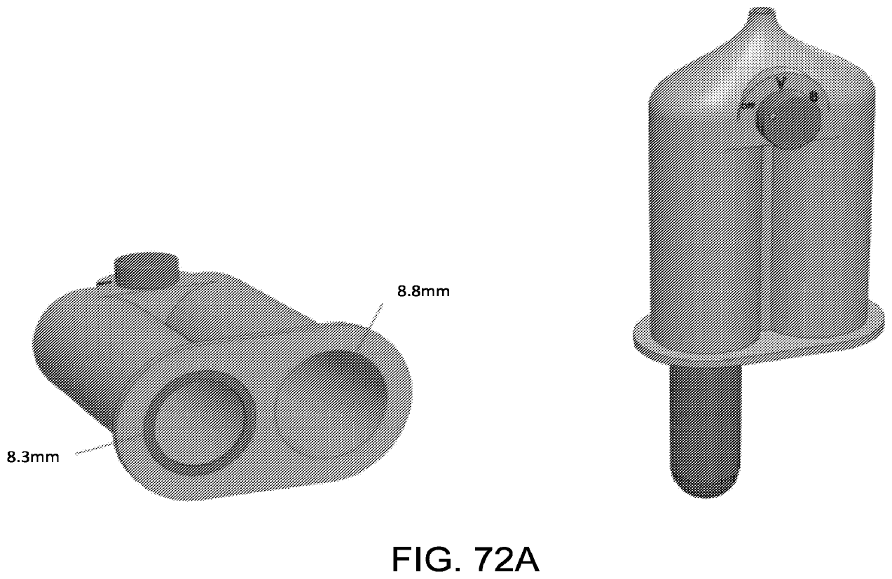

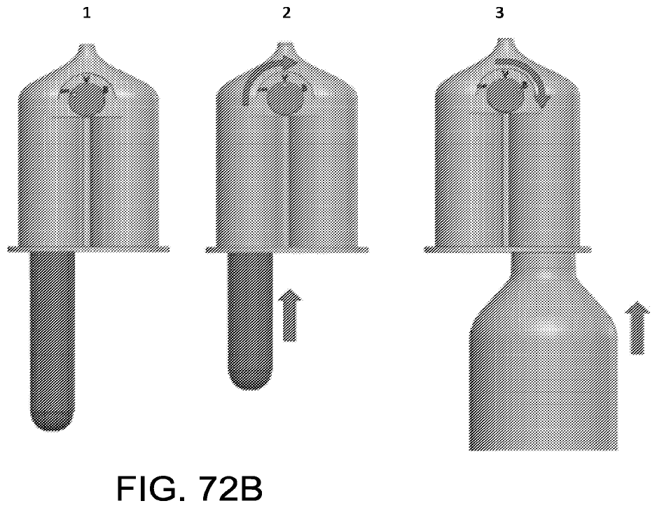

FIG. 72A-FIG. 72B illustrates an alternative aspect of the dual port adaptor. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The adaptor also has a rotary valve that will allow patient sample to flow into the collection/discard tube in one position, and into the blood culture bottle in a second position. Initially, before sample is collected, the rotary valve is in a third position (the “off” position). When the collection/discard tube is brought into fluid communication with the needle in the adaptor, the rotary valve is moved to the first position. Once the initial portion of the patient sample is collected in the collection/discard tube, the collection/discard tube is removed from the adaptor, and the valve is moved to the third position, which allows collecting the patient sample into the blood culture container when the blood culture bottle is brought into fluid communication with the adaptor needle.

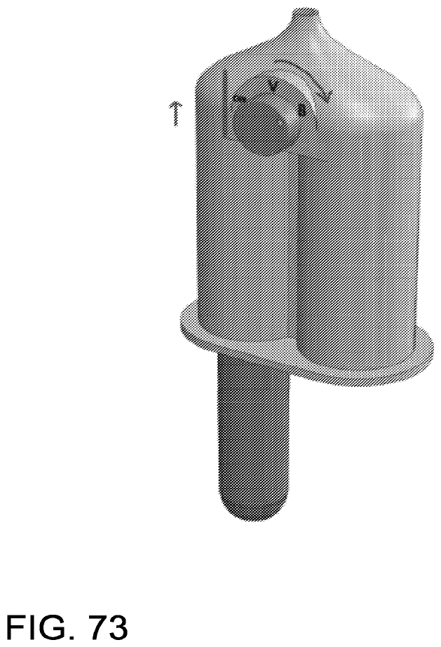

FIG. 73 is an alternative aspect of FIG. 72A-FIG. 72B wherein the adaptor has a locking pin that requires the blood culture bottle to be inserted into the adaptor before the rotary valve is moved to the third position.

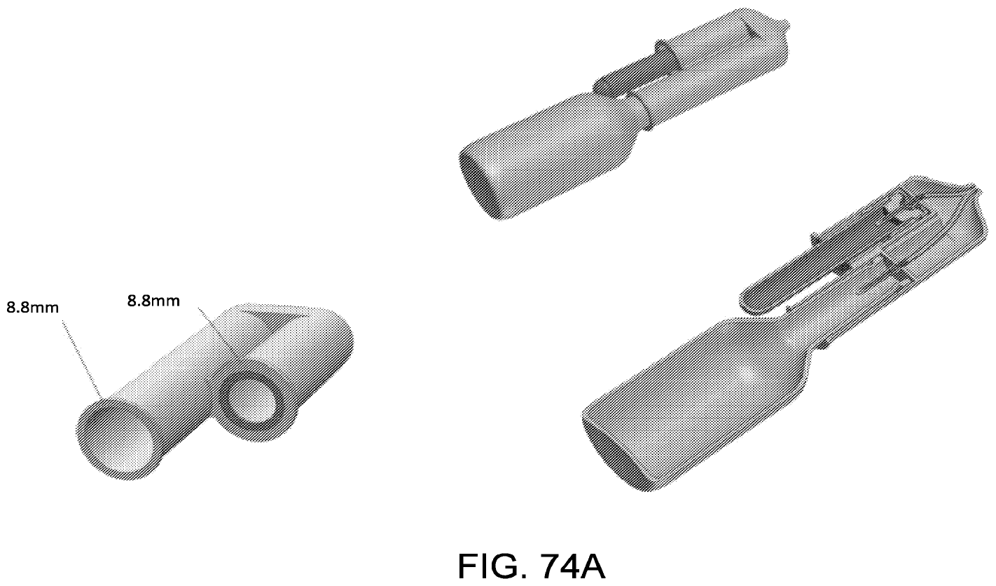

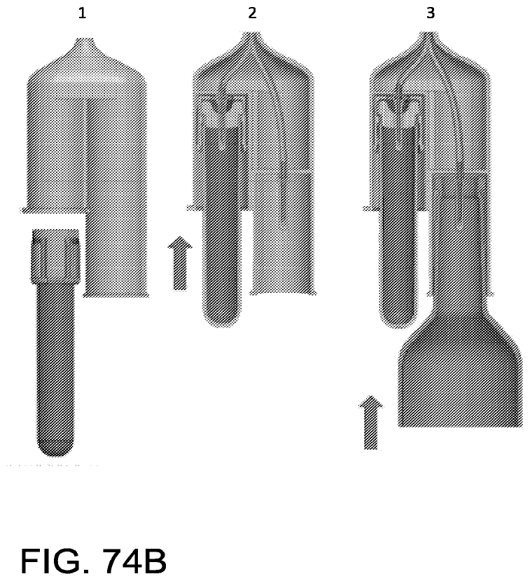

FIG. 74A-FIG. 74B illustrates an alternative aspect of the dual port adaptor. The assembly described herein deploys a collection/discard tube in communication with a dual port adaptor for connection to a patient line set to collect sample from a patient into the container. The collection/discard tube will only fit in one of the dual ports. In this aspect, the collection/discard tube is assembled with the adaptor but held in spaced relation from the adaptor needle during shipment and storage by a ring or other mechanism described herein. The adaptor has the first port for receiving the collection/discard tube and a second port for receiving the blood culture bottle. The ports are staggered so the collection/discard tube can be placed in one port and the culture bottle can be placed in the other port simultaneously and to provide a differential pressure between the flow path to the collection/discard tube and the flow path to the culture bottle. When the collection/discard tube is brought into fluid communication with the needle in the adaptor, sample is collected into the sample collection/discard tube. Once the initial portion of the patient sample is collected in the collection/discard tube, the blood culture bottle is placed in fluid communication with the needle in the second port. Because the second port is lower than the first port (or, more precisely, because the sample collection tube is held higher in the adaptor than the blood culture vessel) the sample flows preferentially to the blood culture vessel when both the collection/discard tube and the culture bottle are connected in the adaptor.

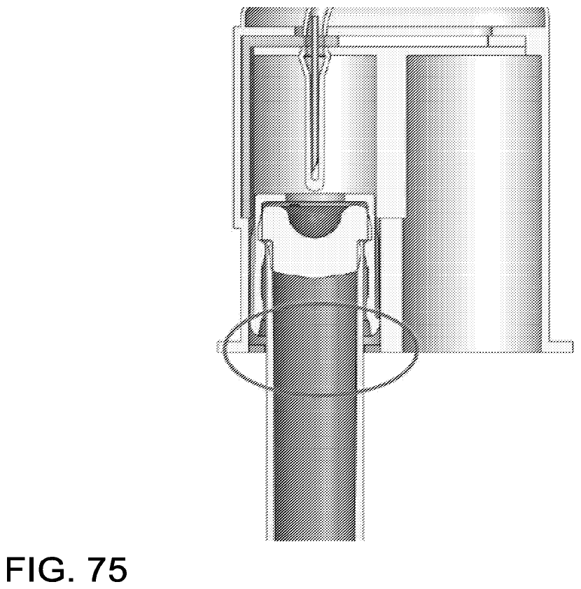

FIG. 75 is an aspect of a dual port adaptor wherein the sample collection/discard tube is held in spaced relation to the needle in the adaptor by variety of mechanisms described herein such as the rubber ring, the clutch, the sticker or other spacing mechanism. FIG. 75 illustrates a generic separation mechanism integrated with the sliding needle shuttle described in FIG. 71.

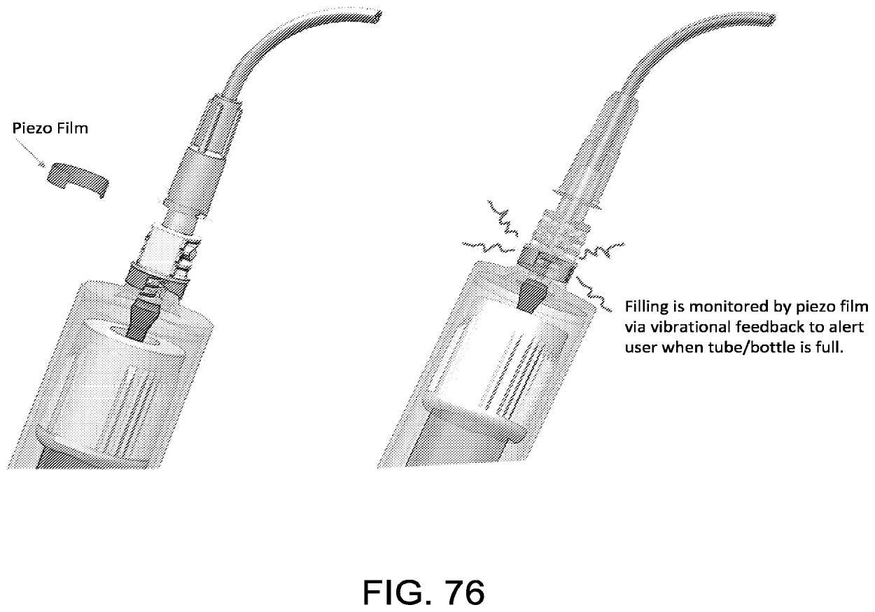

FIG. 76 illustrates an adaptor in fluid communication with a sample collection/discard tube. The adaptor has a piezoelectric flow monitor that will indicate to a use when the sample collection discard tube is contains the target amount of sample.

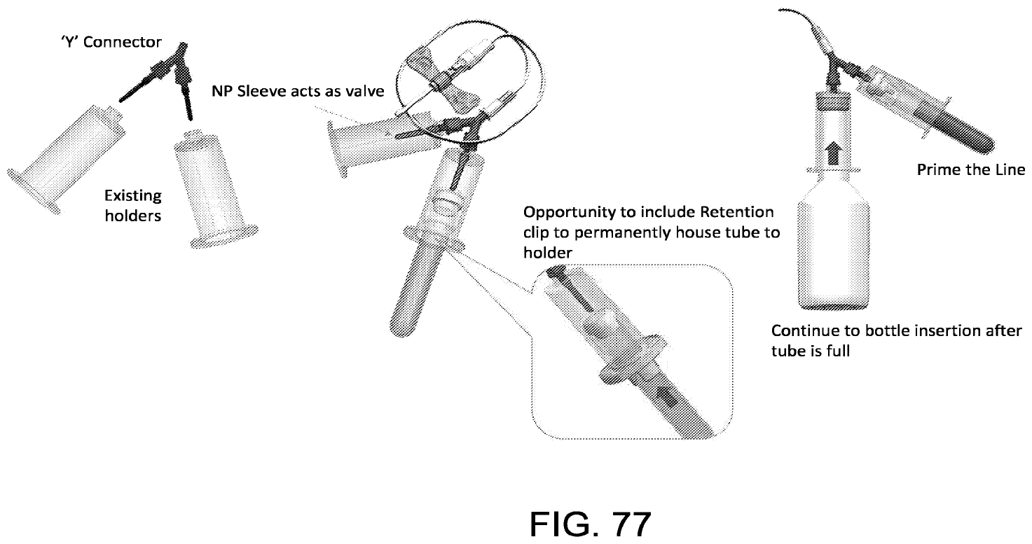

FIG. 77 is an alternative aspect of two port adaptor. Each port is affixed to an end of a “Y” connector. The port that receives the sample collection/discard tube has a retention mechanism that keeps the collection/discard tube in spaced relation from the needle in the adaptor during shipment and storage. When the sample collection/discard tube is brought into fluid communication with the needle in the adaptor port, the line from the line set to the “Y” connector is primed. The sleeve on the needle in the second port acts like a shut off valve when sample is being collected into the sample collection/discard tube. When the culture bottle is placed in fluid communication with the needle in the second port of the adaptor, sample flows preferentially into the culture bottle.

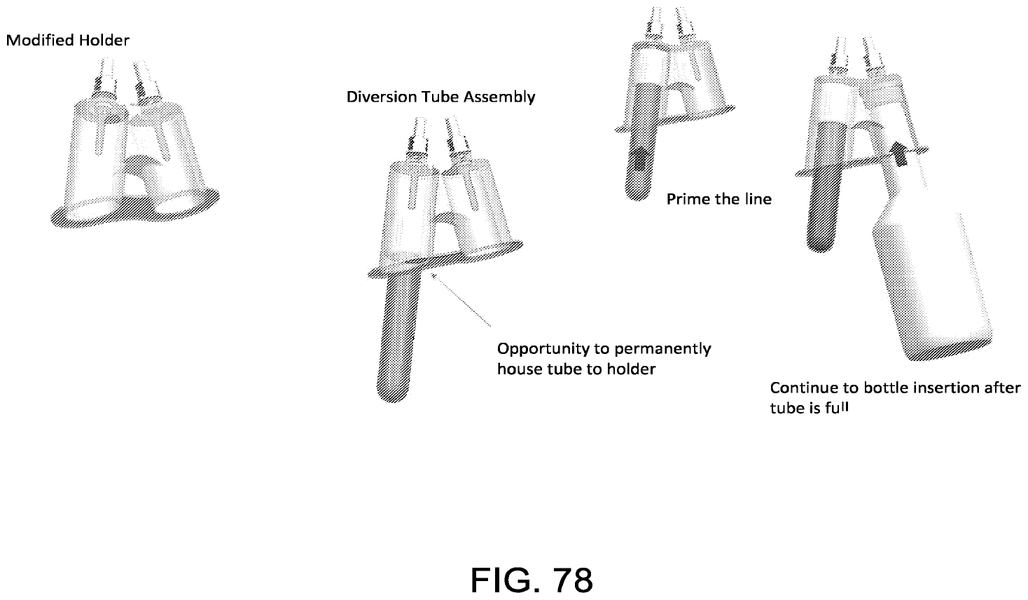

FIG. 78 is an alternative aspect of two port adaptor of FIG. 77 Each port is affixed to an end of a “Y” connector and the ports themselves are arranged in a “Y.” The port that receives the sample collection/discard tube may have a retention mechanism that keeps the collection/discard tube in spaced relation from the needle in the adaptor during shipment and storage. When the sample collection/discard tube is brought into fluid communication with a needle in the adaptor port, the line from the line set to the “Y” connector is primed. The sleeve on the needle in the second port acts like a shut off valve when sample is being collected into the sample collection/discard tube. When the culture bottle is placed in fluid communication with the needle in the second port of the adaptor, sample flows preferentially into the culture bottle since the collection/discard tube has the requisite amount of sample therein.

Sample collection systems are described herein. In one aspect, a sample collection system includes a container for collecting a sample, an adaptor having a distal end, a proximal end, a needle, and an interior. The adaptor is configured to receive a sample through a connector at the distal end. The needle extends from the connector into the interior of the adaptor, and the proximal end includes an opening to receive a first end of the container into the interior of the adaptor. The system also includes a securing mechanism configured to releasably secure the container at a first position, wherein, at the first position, the first end of the container is held in a spaced relationship with the needle in the interior of the adaptor. In one aspect, the securing mechanism is operated to allow the container to be advanced distally within the interior of the adaptor from the first position to a second position and thereby engage the needle to receive the sample into the container. In one aspect, the securing mechanism is further configured to enable the container to be withdrawn proximally to remove the container from the interior of the adaptor. In a further aspect, when the container is removed from the interior of the adaptor, the securing mechanism is configured to be removed by the container.

In one aspect, the securing mechanism comprises a collar fitted on the container. In a further aspect, the collar has an inner diameter that is larger than an outer diameter of the container an outer diameter that is smaller than an inner diameter of the adaptor. In a further aspect, the collar provides an interference fit when advanced into the adaptor. In yet a further aspect, the collar has a flange that prevents the collar from being advance completely into the adaptor.

In another aspect, the securing mechanism is a c-clamp. The securing mechanism may also have a resilient gasket that is received on the container and forms an interference fit for the container within the adaptor.

In another aspect, the container has a cap that may have a septum and a shield. The shield may extend from the septum and surrounding a perimeter of a portion of the container. In a further aspect, the above-described shield may have a first O-ring at a proximal end and a second O-ring at a distal end, each O-ring providing an interference fit when in the adaptor.

In another aspect, the securing mechanism is a collar on the container that forms a snap fit with a base of the shield, the securing mechanism providing an interference fit in the adaptor when advanced into the adaptor. In a further aspect, the collar has a flange at the proximal end thereof to prevent the collar from advancing completely into the adaptor.

In another aspect, the adaptor may have a spring on the interior thereof, which provides resistance when the container is advanced into the adaptor and into engagement with the needle, the spring biased to advance the container out of the adaptor after sample collection.

In another aspect of the sample collection system, the securing mechanism is a flanged foam rubber collar placed on the container that forms an interference fit with the adaptor when advanced into the adaptor, the flange having a diameter that is larger than an inner diameter of the adaptor to prevent the flanged collar from being completely advanced into the adaptor.

In another aspect of the sample collection system, the securing mechanism is a foam rubber collar placed on the container that seats within a flanged opening at the proximal end of the adaptor, the foam rubber collar forming an interference fit with the container but permits the container to be advanced and withdrawn through the collar.

In another aspect of the sample collection system, the securing mechanism is a foam rubber collar secured on the container and forms interference fit when introduced into the adaptor. In a further aspect, the foam rubber collar is secured on the container with adhesive.

In another aspect of the sample collection system, the securing mechanism may be a flanged, O-ring collar on the container, wherein the O-ring forms a friction fit in the adaptor when the container is advanced in the adaptor and wherein the flange does not advance into the adaptor. In this aspect, the container is movably received in the flanged portion.

In another aspect, described herein is a method for collecting a sample from a patient, the method comprising providing a sample collection system as described above, manipulating the securing mechanism of the sample collection system to permit the container to be advanced into fluid communication with the needle in the adaptor, collecting sample into the container and removing the container from the adaptor.

In another aspect, a sample collection system is described that includes a container for collecting a sample, an adaptor comprising a distal end, a proximal end, a needle, and an interior, the adaptor configured to receive a sample through a connector at the distal end, the needle extending from the connector into the interior of the adaptor, and the proximal end including an opening to receive a first end of the container into the interior of the adaptor, and a securing mechanism configured to hold the container at a first position, wherein, at the first position, the first end of the container is held in a spaced relationship with the needle in the interior of the adaptor. In the above aspect, the securing mechanism is configured to allow the container to be advanced distally within the interior of the adaptor from the first position to a second position and thereby engage the needle to receive the sample into the container, wherein the securing mechanism is further configured to enable the container to be withdrawn proximally to remove the container from the interior of the adaptor. Optionally, when the container is removed from the interior of the adaptor, the securing mechanism is configured to be withdrawn by the container.

In another aspect, the securing mechanism is made of rubber and has an annular shape, wherein the securing mechanism is disposed around an exterior of the container, and wherein the securing mechanism has a distal portion comprising a thickness that will fit between the container and the adaptor and a proximal portion comprising a flange having a diameter that exceeds an inner diameter of the adaptor.

In another aspect, the securing mechanism provides a snap fit with a shield for the container, the securing mechanism comprising a rim that extends away from the container and provides a stop that prevents the securing mechanism from being advanced further into the adaptor.

In another aspect, the securing mechanism comprises a compressible spring attached to a distal end of the container, the compressible spring configured to be inserted between the container and the adaptor, wherein the compressible spring holds the container in the first position, but permits the container to be advanced into the second position when a tension provided by the spring is overcome.

In another aspect, the securing mechanism comprises a foam rubber flange that provides an interference fit on the container wherein the container can be advanced in the foam rubber flange with application of a force from the first position to the second position, and wherein a first portion of the foam rubber flange may be advanced into the adaptor and a second portion of the foam rubber flange is too large to be advanced in to the adaptor.

In another aspect, the securing mechanism is a foam rubber flange that is adhesively fixed to a corresponding flange at a base of the adaptor.

In another aspect, the securing mechanism is a collar that forms a friction fit with the container such that the collar cannot be advanced into the adaptor but the container may be advanced through the collar from the first position to the second position.

In another aspect, the securing mechanism is an O-ring secured on the container by a flange formed on the container.

In another aspect, a removable sticker is applied to the adaptor and container to secure the container in the first position.

In another aspect, the securing mechanism is a collar that retains an o-ring that forms and interference fit between the container and the adaptor, wherein the collar is positioned adjacent to a cap on the container.

In another aspect, the securing mechanism comprises a collar that forms an interference fit with both an outer diameter of the container and an inner diameter of the adaptor.

In another aspect, the securing mechanism further comprises a second portion that is a sleeve that fits on the container, wherein the sleeve comprises a flange that engages the collar such that the flange pulls the collar from the adaptor when the container is removed therefrom.

In another aspect, the securing mechanism comprises a collar that forms an interference fit on the container, wherein the securing mechanism has bearings therein.

In another aspect, the securing mechanism comprises a ratcheting collar, wherein the ratcheting collar comprises tabs that grip the container when the container is removed from the adaptor.

In another aspect, the securing mechanism is a one-way collar that fits on the container, the one-way collar comprising a tubular portion and a flange portion, wherein the tubular portion comprises grips on a surface in contact with the container.

In another aspect, the securing mechanism comprises a collar and a detent that engages with a base of the adaptor and a flange that prevents the collar from advancing into the adaptor.

In another aspect, the securing mechanism comprises a collar comprising one-way grips extending upward into the collar that fits on the container, such that the container can only be advanced through the collar in one direction, wherein the securing mechanism further comprises tape adhered to the container and the collar to secure the container in the first position, wherein the tape is removed or torn when the container is advanced to the second position.

In another aspect, the securing mechanism comprises a first collar portion and a second collar portion, each of which form an interference fit with the container, wherein the first collar portion comprises receptacles for barbs on the second collar portion, wherein the first and second collar portions are joined together when the container is advanced into the second position such that first and second collar portions are carried with the container when the container is removed from the adaptor.

In another aspect, the securing mechanism comprises a snap ring comprising projections that extend upward into the adaptor when the snap ring is received by the adaptor, the container carrying a sleeve thereover, the sleeve carrying an O-ring that forms an interference fit between the container and the adaptor when the O-ring is advanced into the adaptor, the sleeve comprising a raised portion that catches on the projections as the container is advanced into the adaptor.

In another aspect, the securing mechanism comprises a flanged sleeve, wherein the sleeve is flexible and wherein there is diametric clearance between the sleeve and the container.

In another aspect, the securing mechanism has a first portion of the two part collar comprising a clip and a second portion comprising a collar that also provides an interference fit on the container, wherein the clip advances past the second portion of the collar when the container is advance to the second position, and the clip pulls the second portion of the collar from the adaptor when the container is removed from the adaptor.

In another aspect, the securing mechanism is a flanged sleeve comprising detents that engage with a base of the adaptor wherein the detents can be rotated out of engagement with the base of the adaptor to remove the container and the securing mechanism from the adaptor.

In another aspect, the container is secured in the first position with removable adhesive tape.

In another aspect, the securing mechanism is a threaded sleeve, wherein the threaded sleeve forms an interference fit with the container and is advanced upward into the adaptor by screwing the sleeve into the adaptor, wherein the container is backed out of the adaptor by unscrewing the sleeve therefrom.

In another aspect, the securing mechanism comprises a collar and a sleeve, wherein the collar comprises guide notches that receive raised guides on the sleeve, the guide notches having detents therein, the detents holding the raised guides in place when the container is in the first position, wherein the sleeve further comprises tabs that are biased outward such that the tabs carry the collar from the adaptor when the container is removed therefrom.

In another aspect, the securing mechanism comprises a collar and a sleeve, wherein the collar comprises clips configured to attach to a base of the adaptor and a tapered inner portion that receives the sleeve, wherein the sleeve has first raised portions that cooperate with detents on an interior of the sleeve to hold the container in the first position and second raised portion that deflects that sleeve whereby the clips disengage from the base, allowing the container to be removed from the adaptor.

In another aspect, the securing mechanism is a collar with posts that cooperates with first lateral and second linear slots that intersect and that are provided in the adaptor, wherein the slots receive the posts to allow the container to be locked in the first position by rotating the container so that the posts on the collar are received in the lateral slot, and, if the container is rotated such that the posts are in the linear slot, then the container can be advanced into the second position or removed from the adaptor.

In another aspect, the securing mechanism is a collar with posts that cooperates with first slanted and second linear slots that intersect and that are provided in the adaptor, wherein the slanted slots receive the posts to allow the container to be locked in the first position, and, to advance the container to the second position, the container is rotated so that the post is in the linear slot, which allows the container to be advanced into the second position or removed from the adaptor.

In another aspect, the securing mechanism comprises a collar with posts that cooperates with a slotted track in the adaptor, wherein the slot track engages the posts at a first end where the track holds the post such that the container is heled in the first position in the adaptor, wherein rotating the container moves the post into a portion f the track that allows the container to be advanced to the second position, wherein rotating the container again advance the post into the portion of the track that allows the container to be removed from the adaptor, the container carrying the collar out of the adaptor.

In a further aspect, the portion of the slot track that holds the container in the first position comprises a detent to hold the post in place.

In another aspect, the securing mechanism comprises a collar and sleeve assembly, the collar comprising a first portion that is received into the adaptor and a flange portion that will not advance into the adaptor, the collar further comprising a biased detent pin comprising a locking detent and a follower detent that locks into a slot provided in the adaptor, the sleeve comprising a slot having an angled portion and a longitudinal portion, wherein the container is locked in the first position by the locking detent when the detent pin is aligned with the angled portion of the slot and wherein the container can be advanced into the second position or removed from the adaptor when the detent pin is aligned with the longitudinal portion of the slot such that the locking detent is no longer held the adaptor slot.

In another aspect, the securing mechanism comprises a collar and sleeve assembly, the collar comprising flexible posts and the sleeve comprising a flange that will not advance into the adaptor, wherein the adaptor comprises deflectable tabs that are positioned to be received by slots in the sleeve to lock the container in the first position in the adaptor, wherein advancing the collar into the container causes the flexible posts to move the deflectable tabs from engagement with the slots in the sleeve, allowing the container to be advanced to the second position or out of the adaptor.

In another aspect, the securing mechanism comprises a sleeve comprising a bump off, first slots, second slots, and a flange that stops the sleeve from advancing completely into the adaptor, and a collar comprising raised portions and flexible projections biased toward an interior of the adaptor into which the container is received, and ribs, wherein the ribs engage the container on which the sleeve is placed, wherein the adaptor comprises projections that are biased inward, wherein the projections engage the sleeve slots to hold the container in the first position and wherein the raised portions of the collar move the projections out of engagement with the sleeve slots to allow the container to advance to the second position and to lock the collar to the sleeve allowing the sleeve and collar to be removed with the container.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A sample collection system comprising:
a container for collecting a sample;
an adaptor comprising a distal end, a proximal end, a needle, and an interior, the adaptor configured to receive a sample through a connector at the distal end, the needle extending from the connector into the interior of the adaptor, and the proximal end including an opening to receive a first end of the container into the interior of the adaptor; and
a securing mechanism configured to releasably secure the container at a first position, wherein, at the first position, the first end of the container is held in a spaced relationship with the needle in the interior of the adaptor,
wherein the securing mechanism is operated to allow the container to be advanced distally within the interior of the adaptor from the first position to a second position and thereby engage the needle to receive the sample into the container,
wherein the securing mechanism is further configured to enable the container to be withdrawn proximally to remove the container from the interior of the adaptor,
wherein, when the container is removed from the interior of the adaptor, the securing mechanism is configured to be removed by the container.

2. The sample collection system of claim 1, wherein the securing mechanism comprises a collar fitted on the container.

3. The sample collection system of claim 2, wherein the collar has an inner diameter that is larger than an outer diameter of the container an outer diameter that is smaller than an inner diameter of the adaptor.

4. The sample collection system of claim 2, wherein the collar provides an interference fit when advanced into the adaptor.

5. The sample collection system of claim 2, wherein the collar has a flange that prevents the collar from being advance completely into the adaptor.

6. The sample collection system of claim 1, wherein the securing mechanism is a c-clamp.

7. The sample collection system of claim 6, wherein the securing mechanism further comprises a resilient gasket that is received on the container and forms an interference fit for the container within the adaptor.

8. The sample collection system of claim 1, wherein the container comprises a cap, the cap comprising a septum and a shield, the shield extending from the septum and surrounding a perimeter of a portion of the container.

9. The sample collection system of claim 8, wherein the securing mechanism is a collar on the container that forms a snap fit with a base of the shield, the securing mechanism providing an interference fit in the adaptor when advanced into the adaptor.

10. The sample collection system of claim 9, wherein the collar has a flange at the proximal end thereof to prevent the collar from advancing completely into the adaptor.

11. The sample collection system of claim 1, wherein the adaptor has a spring on the interior thereof, which provides resistance when the container is advanced into the adaptor and into engagement with the needle, the spring biased to advance the container out of the adaptor after sample collection.

12. The sample collection system of claim 1, wherein the securing mechanism is a flanged foam rubber collar placed on the container that forms an interference fit with the adaptor when advanced into the adaptor, the flange having a diameter that is larger than an inner diameter of the adaptor to prevent the flanged collar from being completely advanced into the adaptor.

13. The sample collection system of claim 1, wherein the securing mechanism is a foam rubber collar placed on the container that seats within a flanged opening at the proximal end of the adaptor, the foam rubber collar forming an interference fit with the container, but permits the container to be advanced and withdrawn through the collar.

14. The sample collection system of claim 1, wherein the securing mechanism is a foam rubber collar secured on the container and forms interference fit when introduced into the adaptor.

15. The sample collection system of claim 14, wherein the foam rubber collar is secured on the container with adhesive.

16. The sample collection system of claim 1, wherein the securing mechanism is a flanged and O-ring collar on the container, wherein the O-ring forms a friction fit in the adaptor when the container is advanced in the adaptor and wherein the flange does not advance into the adaptor, wherein the container is movably received in the flange.

17. The sample collection system of claim 8, wherein the shield has a first O-ring at a proximal end and a second O-ring at a distal end, each O-ring providing an interference fit when in the adaptor.

18. A sample collection system comprising:
a container for collecting a sample;
an adaptor comprising a distal end, a proximal end, a needle, and an interior, the adaptor configured to receive a sample through a connector at the distal end, the needle extending from the connector into the interior of the adaptor, and the proximal end including an opening to receive a first end of the container into the interior of the adaptor; and
a securing mechanism configured to hold the container at a first position, wherein, at the first position, the first end of the container is held in a spaced relationship with the needle in the interior of the adaptor,
wherein the securing mechanism is configured to allow the container to be advanced distally within the interior of the adaptor from the first position to a second position and thereby engage the needle to receive the sample into the container,
wherein the securing mechanism is further configured to enable the container to be withdrawn proximally to remove the container from the interior of the adaptor,
wherein, when the container is removed from the interior of the adaptor, the securing mechanism is configured to be withdrawn by the container.

19. The sample collection system of claim 18, wherein the securing mechanism is made of rubber and has an annular shape, wherein the securing mechanism is disposed around an exterior of the container, and wherein the securing mechanism has a distal portion comprising a thickness that will fit between the container and the adaptor and a proximal portion comprising a flange having a diameter that exceeds an inner diameter of the adaptor.

20. The sample collection system of claim 18, wherein the securing mechanism provides a snap fit with a shield for the container, the securing mechanism comprising a rim that extends away from the container and provides a stop that prevents the securing mechanism from being advanced further into the adaptor.

21. The sample collection system of claim 18, wherein the securing mechanism comprises a compressible spring attached to a distal end of the container, the compressible spring configured to be inserted between the container and the adaptor, wherein the compressible spring holds the container in the first position, but permits the container to be advanced into the second position when a tension provided by the spring is overcome.

22. The sample collection system of claim 18, wherein the securing mechanism comprises a foam rubber flange that provides an interference fit on the container wherein the container can be advanced in the foam rubber flange with application of a force from the first position to the second position, and wherein a first portion of the foam rubber flange may be advanced into the adaptor and a second portion of the foam rubber flange is too large to be advanced in to the adaptor.

23. The sample collection system of claim 18, wherein the securing mechanism is a foam rubber flange that is adhesively fixed to a corresponding flange at a base of the adaptor.

24. The sample collection system of claim 18, wherein the securing mechanism is a collar that forms a friction fit with the container such that the collar cannot be advanced into the adaptor but the container may be advanced through the collar from the first position to the second position.

25. The sample collection system of claim 18, wherein the securing mechanism is an O-ring secured on the container by a flange formed on the container.

26. The sample collection system of claim 25, wherein a removable sticker is applied to the adaptor and container to secure the container in the first position.

27. The sample collection system of claim 18, wherein the securing mechanism is a collar that retains an o-ring that forms and interference fit between the container and the adaptor, wherein the collar is positioned adjacent to a cap on the container.

28. The sample collection system of claim 18, wherein the securing mechanism comprises a collar that forms an interference fit with both an outer diameter of the container and an inner diameter of the adaptor.

29. The sample collection system of claim 28, wherein the securing mechanism further comprises a second portion that is a sleeve that fits on the container, wherein the sleeve comprises a flange that engages the collar such that the flange pulls the collar from the adaptor when the container is removed therefrom.

30. The sample collection system of claim 18, wherein securing mechanism comprises a collar that forms an interference fit on the container, wherein the securing mechanism has bearings therein.

31. The sample collection system of claim 18, wherein the securing mechanism comprises a ratcheting collar, wherein the ratcheting collar comprises tabs that grip the container when the container is removed from the adaptor.

32. The sample collection system of claim 18, wherein the securing mechanism is a one-way collar that fits on the container, the one-way collar comprising a tubular portion and a flange portion, wherein the tubular portion comprises grips on a surface in contact with the container.

33. The sample collection system of claim 18, wherein the securing mechanism comprises a collar and a detent that engages with a base of the adaptor and a flange that prevents the collar from advancing into the adaptor.

34. The sample collection system of claim 18, wherein the securing mechanism comprises a collar comprising one-way grips extending upward into the collar that fits on the container, such that the container can only be advanced through the collar in one direction, wherein the securing mechanism further comprises tape adhered to the container and the collar to secure the container in the first position, wherein the tape is removed or torn when the container is advanced to the second position.

35. The sample collection system of claim 18, wherein the securing mechanism comprises a first collar portion and a second collar portion, each of which form an interference fit with the container, wherein the first collar portion comprises receptacles for barbs on the second collar portion, wherein the first and second collar portions are joined together when the container is advanced into the second position such that first and second collar portions are carried with the container when the container is removed from the adaptor.

36. The sample collection system of claim 18, wherein the securing mechanism comprises a snap ring comprising projections that extend upward into the adaptor when the snap ring is received by the adaptor, the container carrying a sleeve thereover, the sleeve carrying an O-ring that forms an interference fit between the container and the adaptor when the O-ring is advanced into the adaptor, the sleeve comprising a raised portion that catches on the projections as the container is advanced into the adaptor.

37. The sample collection system of claim 18, wherein the securing mechanism comprises a flanged sleeve, wherein the sleeve is flexible and wherein there is diametric clearance between the sleeve and the container.

38. The sample collection system of claim 18, wherein the securing mechanism comprises a two-part collar, a first portion of the two part collar comprising a clip and a second portion comprising a collar that also provides an interference fit on the container, wherein the clip advances past the second portion of the collar when the container is advance to the second position, and the clip pulls the second portion of the collar from the adaptor when the container is removed from the adaptor.

39. The sample collection system of claim 18, wherein the securing mechanism is a flanged sleeve comprising detents that engage with a base of the adaptor wherein the detents can be rotated out of engagement with the base of the adaptor to remove the container and the securing mechanism from the adaptor.

40. The sample collection system of claim 39, wherein the container is secured in the first position with removable adhesive tape.

41. The sample collection system of claim 18, wherein the securing mechanism is a threaded sleeve, wherein the threaded sleeve forms an interference fit with the container and is advanced upward into the adaptor by screwing the sleeve into the adaptor, wherein the container is backed out of the adaptor by unscrewing the sleeve therefrom.

42. The sample collection system of claim 18, wherein the securing mechanism comprises a collar and a sleeve, wherein the collar comprises guide notches that receive raised guides on the sleeve, the guide notches having detents therein, the detents holding the raised guides in place when the container is in the first position, wherein the sleeve further comprises tabs that are biased outward such that the tabs carry the collar from the adaptor when the container is removed therefrom.

43. The sample collection system of claim 18, wherein the securing mechanism comprises a collar and a sleeve, wherein the collar comprises clips configured to attach to a base of the adaptor and a tapered inner portion that receives the sleeve, wherein the sleeve has first raised portions that cooperate with detents on an interior of the sleeve to hold the container in the first position and second raised portion that deflects that sleeve whereby the clips disengage from the base, allowing the container to be removed from the adaptor.

44. The sample collection system of claim 18, wherein the securing mechanism is a collar with posts that cooperates with first lateral and second linear slots that intersect and that are provided in the adaptor, wherein the slots receive the posts to allow the container to be locked in the first position by rotating the container so that the posts on the collar are received in the lateral slot, and, if the container is rotated such that the posts are in the linear slot, then the container can be advanced into the second position or removed from the adaptor.

45. The sample collection system of claim 18, wherein the securing mechanism is a collar with posts that cooperates with first slanted and second linear slots that intersect and that are provided in the adaptor, wherein the slanted slots receive the posts to allow the container to be locked in the first position, and, to advance the container to the second position, the container is rotated so that the post is in the linear slot, which allows the container to be advanced into the second position or removed from the adaptor.

46. The sample collection system of claim 18, wherein the securing mechanism is a collar with posts that cooperates with a slotted track in the adaptor, wherein the slot track engages the posts at a first end where the track holds the post such that the container is heled in the first position in the adaptor, wherein rotating the container moves the post into a portion f the track that allows the container to be advanced to the second position, wherein rotating the container again advance the post into the portion of the track that allows the container to be removed from the adaptor, the container carrying the collar out of the adaptor.

47. The sample collection system of claim 46, wherein the portion of the slot track that holds the container in the first position comprises a detent to hold the post in place.

48. The sample collection system of claim 18, wherein the securing mechanism comprises a collar and sleeve assembly, the collar comprising a first portion that is received into the adaptor and a flange portion that will not advance into the adaptor, the collar further comprising a biased detent pin comprising a locking detent and a follower detent that locks into a slot provided in the adaptor, the sleeve comprising a slot having an angled portion and a longitudinal portion, wherein the container is locked in the first position by the locking detent when the detent pin is aligned with the angled portion of the slot and wherein the container can be advanced into the second position or removed from the adaptor when the detent pin is aligned with the longitudinal portion of the slot such that the locking detent is no longer held the adaptor slot.

49. The sample collection system of claim 18, wherein the securing mechanism comprises a collar and sleeve assembly, the collar comprising flexible posts and the sleeve comprising a flange that will not advance into the adaptor, wherein the adaptor comprises deflectable tabs that are positioned to be received by slots in the sleeve to lock the container in the first position in the adaptor, wherein advancing the collar into the container causes the flexible posts to move the deflectable tabs from engagement with the slots in the sleeve, allowing the container to be advanced to the second position or out of the adaptor.

50. The sample collection system of claim 18, wherein the securing mechanism comprises a sleeve comprising a bump off, first slots, second slots, and a flange that stops the sleeve from advancing completely into the adaptor, and a collar comprising raised portions and flexible projections biased toward an interior of the adaptor into which the container is received, and ribs, wherein the ribs engage the container on which the sleeve is placed, wherein the adaptor comprises projections that are biased inward, wherein the projections engage the first slots of the sleeve to hold the container in the first position and wherein the raised portions of the collar move the projections out of engagement with the first sleeve slots to allow the container to advance to the second position and to lock the collar to the sleeve allowing the sleeve and collar to be removed with the container.

51. The sample collection system of claim 18, wherein the securing mechanism comprises a sleeve that is attached to the container, the sleeve having a larger diameter portion, wherein the adaptor comprises a base portion with windows that receive tabs on a locking oval ring that holds the container with the sleeve thereon in the first position in its relaxed state, as the larger diameter portion of the sleeve is advanced into the locking oval ring, the oval ring expands causing the tabs to withdraw from the windows of the adaptor and attach to the sleeve.
